(19) United States
(12) Patent Application Publication
(10) Pub. No.: US 2009/0042281 A1
(43) Pub. Date: Feb. 12, 2009

(54) COMPOUND PROFILING DEVICES, SYSTEMS, AND RELATED METHODS

(75) Inventors: Jim Yuchen Chang, San Diego, CA (US); James Kevin Mainquist, San Diego, CA (US); Robert Charles Downs, La Jolla, CA (US); Jeremy Scot Caldwell, La Jolla, CA (US); Frederick J. King, Encinitas, CA (US)

Correspondence Address: GENOMICS INSTITUTE OF THE;NOVARTIS RESEARCH FOUNDATION, 10675 JOHN JAY HOPKINS DRIVE, SUITE E225, SAN DIEGO, CA, 92121-1127, US

(73) Assignee: IRM LLC, Hamilton (BM)

(21) Appl. No.: 11/841,880

(22) Filed: Aug. 20, 2007

Related U.S. Application Data
(63) Continuation of application No. 11/387,459, filed on Mar. 22, 2006.
(60) Provisional application No. 60/664,640, filed on Mar. 22, 2005.
(60) Provisional application No. 60/680,132, filed on May 11, 2005.

(51) Int. Cl. C12M 1/34

(52) U.S. Cl. 435/287.2; 435/287.1

(57) ABSTRACT

High throughput compound profiling systems, and related devices and sub-systems that can be used to perform various compound profiling processes are provided. These systems typically include work perimeters that are organized for optimum efficiency and processing accuracy. Further, these systems are readily adaptable for performing a wide array of assays, as many different system components are easily incorporated or interchangeable in a particular system. System components that are provided by the invention include cell culture dissociators, which can be used, e.g., to effect cell wetting, dissociation, and/or agitation applications. In some embodiments, these cell culture dissociators are included as components of automated cell culture passaging stations. Dispensing devices that permit on-the-fly fluid temperature regulation are also provided. In addition, various compound profiling methods, cell dissociation methods, uniform cell concentration dispensing methods, among other processes, are also provided.

### CROSS-REFERENCE TO RELATED APPLICATIONS

This application claims priority to U.S. Provisional Applications 60/664,640, filed Mar. 22, 2005, and 60/680,132, filed May 11, 2005, each of which are hereby incorporated by reference in their entirety.

### COPYRIGHT NOTIFICATION

Pursuant to 37 C.F.R. § 1.71(e), Applicants note that a portion of this disclosure contains material which is subject to copyright protection. The copyright owner has no objection to the facsimile reproduction by anyone of the patent document or patent disclosure, as it appears in the Patent and Trademark Office patent file or records, but otherwise reserves all copyright rights whatsoever.

### BACKGROUND OF THE INVENTION

1. Field of the Invention

The present invention relates generally to compound profiling systems in addition to sub-systems and associated methods.

2. Description of the Related Art

High-throughput screening systems are important analytical tools in the process of discovering and developing new drugs. Drug discovery procedures typically involve synthesis and screening of test or candidate drug compounds against selected targets. Candidate drug compounds are generally small molecules, antibodies, nucleic acids, etc., that have the potential to modulate diseases by affecting given targets. Targets are typically cells, organisms, or biological molecules, including proteins (e.g., enzymes, receptors, etc.) or nucleic acids, which are thought to play roles in the onset or progression of particular diseases. A target is typically identified based on its anticipated role in the progression or prevention of a disease. Recent developments in molecular biology and genomics have led to a dramatic increase in the number of targets available for drug discovery research.

Once a target is identified, a library of compounds is typically selected to screen against the target. Enormous compound libraries have been compiled from natural sources and via various synthetic routes, including multi-step solution- or solid-phase combinatorial synthesis schemes. In fact, many pharmaceutical companies and other institutions have access to libraries that include hundreds of thousands, or even millions, of compounds.

A basic premise for screening larger numbers of compounds against a given target is the increased statistical probability of identifying a “hit,” which is a compound that affects the target. Once identified, hits are generally further profiled or characterized for assorted properties as part of chemical optimization processes. These properties often include potency, specificity, toxicity, affect on metabolism, absorption, among other parameters. This additional characterization is typically very labor-intensive due, at least in part, to the large number of compounds to be tested and to the amount of preparation needed for each individual test, and accordingly, oftentimes represents a bottleneck in drug development processes.

There exists a need for efficient automated compound profiling systems that are accurate, reliable, and flexible. The present invention fulfills these and other needs.

### SUMMARY OF THE INVENTION

The present invention relates generally to high throughput compound profiling. For example, the invention provides systems, and related devices and sub-systems, which can be used to perform various compound profiling processes. These highly automated systems and components are typically more flexible, robust, and efficient than pre-existing systems and system components used, e.g., to perform chemical and biochemical library screening. The systems of the invention typically include work perimeters that are organized for optimum efficiency and processing accuracy. Further, these systems are readily adaptable for performing a wide array of assays, as many different system components are easily incorporated or substituted in a given system. Exemplary system components that are provided by the present invention include cell culture dissociators, which can be used, e.g., to effect cell wetting, dissociation, and/or agitation applications. In certain embodiments, these cell culture dissociators are included as components of automated cell culture passaging stations. Dispensing devices that permit on-the-fly fluid temperature regulation are also provided. In addition, the invention also provides various compound profiling methods, cell dissociation methods, uniform cell concentration dispensing methods, among other processes.

In one embodiment, an automated test reagent profiling system comprises an incubation device adapted to facilitate growth of cells in cell culture containers; an automated cell culture passaging system; and an assaying component configured to perform an assay on cells from said cell cultures, wherein the incubation device is adapted to permit the cells from the cell culture to be directly or indirectly delivered to the assay device without the need for human intervention.

In one aspect, the assaying component comprises a test reagent source region structured to support at least one test reagent source container; an assaying region structured to support at least one cell sample container; and a material transfer device that is configured to transfer at least one test reagent from the test reagent source container to the cell sample container when the test reagent source container is supported in the test reagent source region and the cell sample container is supported in the assaying region. In a further aspect, the system additionally comprises a controller, which controller comprises a logic device. In another aspect, the controller is operably connected to the material transfer device, and the logic device comprises logic instructions that direct movement of the material transfer device between the test reagent source region and the assaying region. In another aspect, either or both of the cell sample container and the test reagent source container are multi-well containers.

In one particular aspect, the test reagents comprise one or more reagents selected from the group consisting of compounds, proteins, nucleic acids, virus particles, and bacteriophage. In another aspect, the test reagents comprise nucleic acids selected from the group consisting of siRNA molecules, antisense RNA molecules, cDNAs, and vectors. In another aspect, the test reagents comprise proteins selected from the group consisting of enzymes, antibodies, and regulatory proteins. In another aspect, the test reagents comprise virus particles selected from the group consisting of baculovirus, retrovirus, lentivirus, and adenovirus.

In one aspect, the system further comprises at least one detector configured to detect one or more detectable signals produced in the cell sample container. In another aspect, the material transfer device comprises a non-pressure-based material transfer probe. In a further aspect, the non-pressure-based material transfer probe comprises a pin tool. In a yet further aspect, the material transfer device comprises at least one chassis and the pin tool comprises a support structure having at least one attachment feature that removably attaches to the chassis. In a still further aspect, the logic device comprises logic instructions that directs the material transfer device to attach and/or detach the pin tool to or from the chassis.

In another further aspect, the pin tool comprises a pin tool head having a rotational adjustment feature such that the pin tool head is capable of rotating relative to the support structure along one or more axes.

In one aspect, the test reagent source region and/or the assaying region comprises a container positioning device, which container positioning device comprises at least one container station that is structured to position at least one container relative to the material transfer device. In another aspect, the container station is structured to position at least one multi-well container that comprises 6, 12, 24, 48, 96, 192, 384, 768, 1536, 3456, 9600, or more wells. In another aspect, the container station is structured to rotate relative to the material transfer device.

In an aspect, the system further comprises at least one material transfer probe washing station that comprises at least one wash reservoir structured to wash the non-pressure-based material transfer probe. In a further aspect the wash reservoir comprises at least one mount to position the non-pressure-based material transfer probe relative to the wash reservoir when the non-pressure-based material transfer probe is washed and/or when the non-pressure-based material transfer probe is separated from a chassis of the material transfer device.

In one aspect, the system also comprises a first chamber that comprises a system component disposed therein; a second chamber that communicates with the first chamber such that one or more containers are capable of being translocated between the first and second chambers; and a decontamination component that communicates at least with the second chamber, which decontamination component is configured to substantially decontaminate one or more surfaces of the containers when the containers are disposed in the second chamber. In a further aspect, the system component comprises a cell culture dissociator, a material handling component, and/or a container positioning device.

In one aspect, the system also includes a translocation mechanism that is structured to translocate at least one container at least between the first and second chambers. In another aspect, the first chamber comprises a substantially sterile environment. In another aspect, the second chamber comprises an ante-chamber. In another aspect, the decontamination component comprises at least one radiation source that irradiates the surfaces of the containers to substantially decontaminate the surfaces when the containers are disposed in the second chamber. In another aspect, the decontamination component comprises at least one temperature modulator that modulates temperatures in the second chamber to substantially decontaminate the surfaces when the containers are disposed in the second chamber. In another aspect, the decontamination component comprises at least one decontamination fluid mister that sprays a mist of a decontamination fluid onto the surfaces of the containers to substantially decontaminate the surfaces when the containers are disposed in the second chamber. In another aspect, the decontamination component comprises at least one gas source that flows gas into the second chamber at velocities that are sufficient to substantially remove at least one contaminant from one or more surfaces of the containers when the containers are disposed in the second chamber. In a further aspect, the gas comprises air.

In one aspect, the system also includes a controller and one or more additional system components operably connected to the controller, which additional system components are selected from the group consisting of: a robotic gripping device, a material handling component, a cell counting device, a centrifuge, a detector, a freezer, a fermentor, a waste container, a filtration device, a lid processing device, a transfer station, an incubation device, a colony picking device, a high content imaging device, a pin tool drying or blotting station, a cell dissociator, and a container storage device. In a further aspect, the system comprises at least one container location database operably connected to the controller, which container location database comprises entries that correspond to locations of containers in the system.

In one aspect, the system also includes a material handling component, wherein the material handling component comprises at least one fluidic material transfer component that is configured to transfer fluidic materials to and/or from containers positioned in one or more components of the system. In another aspect, the fluidic material transfer component is configured to transfer cell culture media among cell culture sample vessels, cell culture flasks, and/or multi-well containers. In yet another aspect, the system also includes a controller, which controller comprises a logic device, wherein the logic device comprises at least one logic instruction for pooling separate first cell culture media from m first cell culture containers in n second containers to produce pooled cell culture media using the fluidic material transfer component, wherein m is an integer greater than one, and wherein n is an integer greater than zero and less than m; and transferring selected volumes of the pooled cell culture media from the n second containers into selected wells of p multi-well containers using the fluidic material transfer component, wherein p is an integer greater than one.

In a further aspect, the system includes at least one detection component operably connected to the controller, which detection component is configured to detect a concentration of cells in or from the pooled cell culture media.

In another aspect the fluidic material transfer component comprises a dispensing device that comprises a conduit that comprises an inlet and an outlet that fluidly communicate with one another; a fluid source that fluidly communicates with the inlet of the conduit; a fluid conveyance device operably connected to the conduit and/or to the fluid source, which fluid conveyance device is configured to convey at least one fluidic reagent through the conduit from the fluid source; and a thermal regulation component that thermally communicates with at least a portion of the conduit, which thermal regulation component is configured to selectively regulate a temperature of the fluidic reagent when the fluidic reagent is conveyed through the conduit from the fluid source.

In a further aspect, the system also includes a fluid source storage device that stores the fluid source at a selected temperature. In a yet further aspect, the selected temperature is about 4° C. In one aspect, the system additionally comprises at least one dispense head that comprises at least a segment of the conduit. In a further aspect, the segment of the conduit comprises a coiled structure. In another further aspect, the system also includes comprising a plurality of conduits, wherein the dispense head comprises one or more segments of each of the conduits. IN a still further embodiment, the system also includes a plurality of fluid sources, wherein each of the conduits fluidly communicates with a different fluid source. In one aspect, the dispense head comprises at least one chamber that comprises the segment of the conduit, which chamber comprises at least one opening that fluidly communicates with the thermal regulation component, which thermal regulation component is configured to flow at least one fluidic material having a selected temperature into the chamber such that when the fluidic reagent is flowed through the segment of the conduit, the fluidic reagent substantially attains the selected temperature. In a further aspect, the fluidic material comprises an antifreeze solution. In another further aspect, the selected temperature is about 37° C. In another further aspect, the thermal regulation component comprises at least one fluidic material recirculation bath that substantially maintains the fluidic material at the selected temperature.

In one aspect, the system also comprises at least one high throughput processing station that comprises at least one rotational robot that comprises a reach that defines a work perimeter associated with the rotational robot, wherein at least the cell culture device is within the reach of the rotational robot. In another aspect, the system further comprises a robotic arm that can transfer cell culture containers between the cell culture device and the assay device. In a further aspect, the system also includes at least a second robotic arm.

In one aspect, the automated cell culture passaging system can split or subculture two or more cell lines without human intervention. In a further aspect, the automated cell culture passaging system can split or subculture 25 or more cell lines without human intervention. In another further aspect, the system further comprises a cell dissociator comprising a container holder comprising a container receiving area that is structured to receive at least one cell culture container; a moving mechanism operably connected to the container holder, which mechanism is configured to move the container holder between a first position and a second position; and a stop that limits movement of the container holder by the moving mechanism; a material handling component; and a controller operably connected to the cell culture dissociator and to the material handling component, which controller comprises a logic device that comprises logic instructions that direct the moving mechanism to move the container holder at a selected rate, and the material handling component to dispense material into, and/or to remove material from, the cell culture container when the cell culture container is disposed in the container receiving area.

In a further aspect, the moving mechanism comprises a rotational mechanism, which rotational mechanism is configured to rotate the container holder about an axis; the stop limits angular displacement of the container holder by the rotational mechanism; and the logic instructions direct the rotational mechanism to rotate the container holder at a selected rate. In a still further aspect, the rotational mechanism comprises a counterweight that counters a weight of the container holder when the rotational mechanism rotates the container holder. In another further aspect the cell culture dissociator comprises multiple container holders, which container holders are symmetrically positioned relative to a rotatational axis such that the container holders counterbalance one another. In another further aspect the rotational mechanism comprises a first stop that limits the angular displacement of the container holder in a first direction, and a second stop that limits the angular displacement of the container holder in a second direction that is opposite to the first direction.

In another further aspect, the selected rate is an angular velocity of at least 0.25 rev/s when the stop is contacted. In another further aspect, the container holder decelerates at a rate of at least 1.0 rev/s² when the stop is contacted. In another further aspect, the container holder is structured to receive a cell culture container that comprises a top wall, which top wall comprises a major axis and a minor axis, and the rotational mechanism rotates the container holder in a first direction and an opposite second direction that are parallel to a minor axis of the top wall of the cell culture container. In another further aspect, the container holder is structured to receive cell culture container that comprises a top wall, which top wall comprises a major axis and a minor axis, and the rotational mechanism rotates the container holder in a first direction and an opposite second direction that are parallel to a major axis of the top wall of the cell culture container.

In another further aspect, the system further comprises at least one container retention component that is movable relative to the container holder, which container retention component is structured to retain the cell culture container in a substantially fixed position relative to the container retention component when the cell culture container is disposed in the container receiving area and the container holder is in a closed position. In a still further aspect, the container holder and the container retention component are coupled to one another via at least one slidable coupling. In another still further aspect, the logic device comprises at least one logic instruction that directs the container holder to close or open. In another still further aspect, the container retention component comprises a container retention plate. In another still further aspect, the container retention component is structured to permit access to the cell culture container when the cell culture container is disposed in the container receiving area and the container holder is in the closed position.

In one aspect, the system further includes a multicontainer holder that comprises a plurality of container holders, which multicontainer holder is not operably connected to the moving mechanism. In a further aspect, the logic device comprises at least one logic instruction that directs the container holders to close or open. In a further aspect, the system also comprises at least one translational mechanism operably connected to the multicontainer holder, which translational mechanism is configured to move the multicontainer holder along at least one translational axis. In a yet further aspect, the controller is operably connected to the translational mechanism and comprises at least one logic instruction that directs the translational mechanism to translate the multicontainer holder to one or more selected positions along the translational axis.

In another embodiment, an automated method of passaging a cell culture and performing an assay comprises transferring a portion of a cell culture media located within a source container to a daughter flask; dispensing at least a portion of the cell culture media located within the daughter container to an assay container; and performing an assay on the portion of the cell culture media located within the assay container, wherein the steps of transferring a portion of a cell culture media located within the source container to the daughter container, transferring a portion of a cell culture media located within the daughter container to an assay container, and performing the assay are done without human intervention.

In one aspect, dispensing at least a portion of the cell culture media located within the daughter container to an assay container comprises dispensing an aliquot of the cells of the cell culture media into one or more wells of a multi-well container; and performing an assay on the portion of the cell culture media located within the assay container comprises dispensing a test reagent into a well in the multi-well container; and detecting an effect of the test reagent on the cells. In a further aspect, a plurality of source containers comprise cells from different cell lines or the same cell line, and an aliquot of the cells from each of the cell lines are dispensed into one or more wells of the multi-well container. In a still further aspect, upon completion of depositing an aliquot of the cells from each of the cell lines into one or more wells of the multi-well container, all cell-containing wells of a particular multi-well container contain cells of the same cell line. In another further aspect, upon completion of depositing an aliquot of the cells from each of the cell lines into one or more wells of the multi-well container, a particular multi-well container comprises wells that contain cells from a first cell line and wells that contain cells from at least a second cell line.

In one aspect, the test reagent comprises one or more reagent selected from the group consisting of compounds, nucleic acids, proteins, viruses, and bacteriophage. In one aspect, fewer than 5,000 test reagents are profiled against the cells. In one aspect, 5,000 or more test reagents are profiled against the cells. In one aspect, the effect of the test reagent on the cells is a stimulation or inhibition of one or more of cell proliferation, cell death, translocation, and protein synthesis.

In one aspect, dispensing at least a portion of the cell culture media located within the source container to the daughter container comprises transferring at least a portion of the cell culture media located within the source container to a plurality of daughter containers. In one aspect, the method further comprises performing a non-intrusive cell count of the cell culture media prior to transferring a portion of the cell culture media located within the source container to the daughter container. In another aspect, the method further comprises agitating the source container prior to transferring a portion of the cell culture media located within the source container to the daughter container. In a further aspect, the source container is agitated by a robot arm.

In one aspect, the cell concentration in the cell culture media is determined prior to transferring a portion of the cell culture media located within the source container to the daughter container. In a further aspect, a volume of the portion of the cell culture media that is transferred to the daughter container is calculated based on the cell concentration.

In one aspect, transferring a portion of the cell culture media located within the source container to the daughter container comprises pooling separate first cell culture media from m source containers in n daughter containers to produce pooled cell culture media, wherein m is an integer greater than one, and n is an integer greater than zero and less than m; and transferring selected volumes of the pooled cell culture media from the daughter cell culture containers into selected wells of p assay containers, wherein the assay containers comprise multi-well containers, and wherein p is an integer greater than zero, thereby dispensing the cell culture medium into aliquots having substantially uniform cell concentrations. In a further aspect, m equals p. In another further aspect, m equals an integer from 2 to 100 inclusive. In another further aspect, p equals an integer from 2 to 100 inclusive. In another further aspect, a ratio of m:n is between about 1:1 and about 100:1.

In another further aspect, the method further comprises determining a concentration of cells in a pooled cell culture medium contained in at least one of the daughter containers. In another further aspect, pooling separate cell culture media from m source containers in n destination containers comprises transferring volumes of cell culture media from at least one of the source containers to at least two of the daughter containers. In another further aspect, transferring selected volumes of the pooled cell culture media from the daughter cell culture containers into selected wells of p multi-well containers comprises transferring substantially identical volumes of the pooled cell culture media from the daughter containers into substantially all wells of the multi-well containers. In another further aspect, the source containers each comprise a volume capacity of about 10 mL. In another further aspect the daughter containers each comprise a volume capacity of about 100 mL. In another further aspect, cells of the first cell culture media comprise a single cell line. In another further aspect, the multi-well containers each comprise 6, 12, 24, 48, 96, 192, 384, 768, 1536, 3456, 9600, or more wells. In another further aspect, the wells of at least one of the multi-well containers together comprise a volume capacity of about 10 mL. In another further aspect, n comprises an integer greater than one and (a) comprises transferring substantially equal volumes from at least one of the source containers into each of the daughter containers. In a still further aspect, the substantially equal volumes comprise about 5 mL.

In one aspect, the method further includes dissociating the cells of the cell culture media from each other and/or from a container prior to transferring the cell culture media from the container. In a further aspect, dissociating the cells of the cell culture media comprises placing the container into a container holder of a cell culture dissociator; moving the container holder in a first direction until a first stop is contacted, which first stop limits the displacement of the container holder in the first direction; and moving the container holder in a second direction, which second direction is opposite to the first direction, until a second stop is contacted, which second stop limits the displacement of the container holder in the second direction. In another further aspect, dissociating the cells of the cell culture media comprises rotating the container holder in a first direction until a first stop is contacted, which first stop limits the angular displacement of the container holder in the first direction; and rotating the container holder in a second direction, which second direction is opposite to the first direction, until a second stop is contacted, which second stop limits the angular displacement of the container holder in the second direction. In a still further embodiment, the method also includes dispensing at least one dissociative reagent into the source container before, during, and/or after placing the container into the container holder. In another further embodiment, the method also comprises expanding the cell culture by, transferring a portion of the disassociated cells into each of one or more destination containers.

In one aspect, the invention provides a system that includes at least one cell culture dissociator. Although the system is optionally adapted to perform many different processes, in some embodiments the system is configured to perform high throughput compound profiling processes. The cell culture dissociator includes a container holder comprising a container receiving area that is structured to receive at least one cell culture container. The cell culture dissociator also includes a rotational mechanism operably connected to the container holder. The rotational mechanism is configured to rotate the container holder about an axis. In some embodiments, the rotational mechanism comprises a counterweight that counters a weight of the container holder when the rotational mechanism rotates the container holder. Optionally, the cell culture dissociator comprises multiple container holders, which container holders are symmetrically positioned relative to a rotatational axis such that the container holders counterbalance one another. In addition, the cell culture dissociator also includes a stop that limits angular displacement of the container holder by the rotational mechanism. The system also includes a material handling component, and a controller operably connected to the cell culture dissociator and to the material handling component. The controller comprises a logic device that comprises logic instructions that direct the rotational mechanism to rotate the container holder at a selected rate, and the material handling component to dispense material into, and/or to remove material from, the cell culture container when the cell culture container is disposed in the container receiving area. Typically, one or more components of the system are automated.

In certain embodiments, the rotational mechanism described herein comprises a first stop that limits the angular displacement of the container holder in a first direction, and a second stop that limits the angular displacement of the container holder in a second direction that is opposite to the first direction. Typically, the selected rate is an angular velocity of at least 0.25 rev/s when the stop is contacted, and the container holder decelerates at a rate of at least 1.0 rev/s² when the stop is contacted. For example, when stops are contacted, the rotation of the containers disposed the container holders is typically brought to an abrupt or hard stop. In general, impact forces need to transmit shear forces that are larger than the attachment forces holding cells or other materials to container surfaces.

The container holder described herein is generally structured to receive a cell culture container that comprises a top wall. The top wall typically comprises a major axis and a minor axis. In some embodiments, the rotational mechanism rotates the container holder in a first direction and an opposite second direction that are parallel to a minor axis of the top wall of the cell culture container. In other embodiments, the rotational mechanism rotates the container holder in a first direction and an opposite second direction that are parallel to a major axis of the top wall of the cell culture container.

In some embodiments, the system includes a container retention component (e.g., a container retention plate, etc.) that is movable relative to the container holder. For example, the container holder and the container retention component are coupled to one another via a slidable coupling in certain embodiments. The container retention component is structured to retain the cell culture container in a substantially fixed position relative to the container retention component when the cell culture container is disposed in the container receiving area and the container holder is in a closed position. The container retention component is optionally structured to permit access to the cell culture container when the cell culture container is disposed in the container receiving area and the container holder is in the closed position. Typically, the logic device comprises logic instructions that direct the container holder to close or open.

In certain exemplary embodiments, the material handling component comprises a fluidic material transfer component that is configured to transfer fluidic materials to and/or from containers positioned in one or more components of the system. To illustrate, the fluidic material transfer component is typically configured to transfer cell culture media or other reagents between cell culture sample vessels, cell culture flasks, multi-well containers, and/or the like. In these embodiments, the logic device comprises logic instructions for pooling separate first cell culture media from m first cell culture containers in n second containers to produce pooled cell culture media using the fluidic material transfer component in which m is an integer greater than one, and n is an integer greater than zero and less than m. The logic device also generally includes at least one logic instruction for transferring selected volumes of the pooled cell culture media from the n second containers into selected wells of p multi-well containers using the fluidic material transfer component in which p is an integer greater than one. Typically, the system includes a detection component operably connected to the controller. For example, the detection component is configured to detect a concentration of cells in or from the pooled cell culture media in some embodiments.

In some embodiments, the fluidic material transfer component comprises a dispensing device that includes a conduit that comprises an inlet and an outlet that fluidly communicate with one another, and a fluid source that fluidly communicates with the inlet of the conduit. Optionally, a fluid source storage device that stores the fluid source at a selected temperature (e.g., about 4° C., etc.). In these embodiments, the fluidic material transfer component also typically includes a fluid conveyance device operably connected to the conduit and/or to the fluid source. The fluid conveyance device is generally configured to convey a fluidic reagent through the conduit from the fluid source. In addition, the fluidic material transfer component also typically includes a thermal regulation component that thermally communicates with at least a portion of the conduit. The thermal regulation component is generally configured to selectively regulate a temperature of the fluidic reagent when the fluidic reagent is conveyed through the conduit from the fluid source. In certain embodiments, a dispense head that comprises at least a segment of the conduit. The segment of the conduit typically comprises a coiled structure. Typically, the system includes a plurality of conduits in which the dispense head comprises one or more segments of each of the conduits. In some of these embodiments, the system also includes a plurality of fluid sources in which each of the conduits fluidly communicates with a different fluid source. Optionally, the dispense head comprises at least one chamber that comprises the segment of the conduit. The chamber generally comprises at least one opening that fluidly communicates with the thermal regulation component. Further, the thermal regulation component is typically configured to flow a fluidic material (e.g., an antifreeze solution, etc.) having a selected temperature (e.g., about 37° C., etc.) into the chamber such that when the fluidic reagent is flowed through the segment of the conduit, the fluidic reagent substantially attains the selected temperature. In certain of these embodiments, the thermal regulation component comprises a fluidic material recirculation bath that substantially maintains the fluidic material at the selected temperature.

In certain embodiments, the system includes at least one translational mechanism operably connected to the cell culture dissociator. The translational mechanism is typically configured to move the cell culture dissociator along at least one translational axis. In these embodiments, the controller is generally operably connected to the translational mechanism and comprises logic instructions that direct the translational mechanism to translate the cell culture dissociator to one or more selected positions along the translational axis.

The system typically includes one or more additional system components operably connected to the controller. In certain embodiments, for example, the additional system components are selected from, e.g., a robotic gripping device, a cell counting device, a centrifuge, a detector, a freezer, a fermentor, a waste container, a filtration device, a lid processing device, a transfer station (e.g., handoff nests, etc.), an incubation device, a container storage device, a colony picking device, a high content imaging device, a pin tool drying or blotting station, etc. In some embodiments, the system includes a high throughput processing station that comprises at least one rotational robot that comprises a reach that defines a work perimeter associated with the rotational robot in which at least the cell culture dissociator is within the reach of the rotational robot. To further illustrate, the system optionally includes a robotic arm that can transfer cell culture containers between the cell culture dissociator and to and from an incubation device. In some of these embodiments, the system further includes at least a second robotic arm. Optionally, the system includes a container location database operably connected to the controller. The container location database generally comprises entries that correspond to locations of containers in the system.

To further illustrate, the system includes a multicontainer holder that comprises a plurality of container holders in some embodiments. Typically, the multicontainer holder is not operably connected to the rotational mechanism. In some embodiments, the logic device includes logic instructions that direct the container holders to close or open. Optionally, the system includes a translational mechanism operably connected to the multicontainer holder. The translational mechanism is configured to move the multicontainer holder along at least one translational axis. In some of these embodiments, the controller is operably connected to the translational mechanism and comprises at least one logic instruction that directs the translational mechanism to translate the multicontainer holder to one or more selected positions along the translational axis.

In some embodiments, the system includes an assaying component that includes a test reagent source region structured to support at least one test reagent source container, and an assaying region structured to support at least one cell sample container. Either or both of the test reagent source container and the cell sample container are, in some embodiments, multi-well containers. In some embodiments, the test reagents comprise one or more reagents selected from, e.g., compounds, proteins, nucleic acids, virus particles, bacteriophage, etc. Optionally, the test reagents comprise nucleic acids selected from, e.g., siRNA molecules, antisense RNA molecules, cDNAs, vectors, and the like. In certain embodiments, the test reagents comprise proteins selected from, e.g., enzymes, antibodies, regulatory proteins, etc. To further illustrate, the test reagents optionally comprise virus particles selected from, e.g., baculovirus, retrovirus, lentivirus, adenovirus, and the like. The assaying component also typically includes a material transfer device that is configured to transfer at least one test reagent from the test reagent source container to the cell sample container when the test reagent source container is supported in the test reagent source region and the cell sample container is supported in the assaying region. In these embodiments, the controller is generally operably connected to the material transfer device, and the logic device typically includes logic instructions that direct movement of the material transfer device between the test reagent source region and the assaying region. Typically, the system also includes at least one detector configured to detect one or more detectable signals produced in the cell sample container.

In embodiments of the system that include the assaying component, the material transfer device can comprise a non-pressure-based material transfer probe. In some of these embodiments, the non-pressure-based material transfer probe includes a pin tool, e.g., having 6, 12, 24, 48, 96, 192, 384, 768, 1536, 3456, 9600, or more pins. Optionally, the material transfer device comprises a chassis and the pin tool comprises a support structure having at least one attachment feature that removably attaches to the chassis. Typically, the logic device comprises logic instructions that direct the material transfer device to attach and/or detach the pin tool to or from the chassis. In certain embodiments, the pin tool comprises a pin tool head having a rotational adjustment feature such that the pin tool head is capable of rotating relative to the support structure along one or more axes. To further illustrate, the pin tool head is optionally removably attached to the support structure by one or more attachment components.

In addition, the test reagent source region and/or the assaying region of assaying component typically comprises a container positioning device. The container positioning device generally comprises a container station that is structured to position at least one container relative to the material transfer device. For example, the container station is optionally structured to position a multi-well container that comprises 6, 12, 24, 48, 96, 192, 384, 768, 1536, 3456, 9600, or more wells. In some embodiments, the container station is structured to rotate relative to the material transfer device.

In certain embodiments, the assaying component includes a material transfer probe washing station that comprises a wash reservoir structured to wash the non-pressure-based material transfer probe. In some of these embodiments, the wash reservoir comprises a mount to position the non-pressure-based material transfer probe relative to the wash reservoir when the non-pressure-based material transfer probe is washed and/or when the non-pressure-based material transfer probe is separated from a chassis of the material transfer device.

In some embodiments, the system includes a decontamination device (e.g., an air lock decontamination device, etc.) that comprises a first chamber that includes at least one system component (e.g., the cell culture dissociator, the material handling component, a container positioning device, and/or the like) disposed therein, and a second chamber (e.g., an ante-chamber, etc.) that communicates with the first chamber such that one or more containers are capable of being translocated between the first and second chambers. The first chamber generally comprises a substantially sterile environment. Typically, the second chamber communicates with the first chamber via a passageway. The passageway optionally comprises a movable sealing mechanism (e.g., an air lock, etc.) that is structured to reversibly separate the first and second chambers from one another. In addition, the decontamination device also includes a decontamination component that communicates at least with the second chamber. The decontamination component is typically configured to substantially decontaminate one or more surfaces of the containers when the containers are disposed in the second chamber. Typically, the decontamination component includes a translocation mechanism that is structured to translocate a container at least between the first and second chambers.

Essentially any decontamination component is optionally adapted for use with the decontamination device. To illustrate, the decontamination component comprises a radiation source (e.g., a UV light source, etc.) that irradiates the surfaces of the containers to substantially decontaminate the surfaces when the containers are disposed in the second chamber in some embodiments. Optionally, the decontamination component comprises at least one decontamination fluid mister that sprays a mist of a decontamination fluid (e.g., ethanol, etc.) onto the surfaces of the containers to substantially decontaminate the surfaces when the containers are disposed in the second chamber. In some embodiments, the decontamination component comprises at least one temperature modulator that modulates temperatures in the second chamber to substantially decontaminate the surfaces when the containers are disposed in the second chamber. To further illustrate, the decontamination component comprises a gas source that flows gas (e.g., air, an inert gas, etc.) into the second chamber at velocities that are sufficient to substantially remove at least one contaminant from one or more surfaces of the containers when the containers are disposed in the second chamber in certain embodiments. Other exemplary decontamination components that are optionally utilized include, e.g., UV lamps, thermal decontamination devices, plasma cleaning devices, or the like.

In another aspect, the invention provides a cell culture dissociator that includes a container holder comprising at least one container receiving area that is structured to receive at least one cell culture container. Typically, the container holder comprises one or more angled surfaces that guide the cell culture container into the container receiving area when the cell culture container is placed into the container receiving area. The cell culture dissociator also includes a rotational mechanism operably connected to the container holder. The rotational mechanism is configured to rotate the container holder about an axis. In some embodiments, the rotational mechanism comprises a counterweight that counters a weight of the container holder when the rotational mechanism rotates the container holder. Optionally, the cell culture dissociator comprises multiple container holders, which container holders are symmetrically positioned relative to a rotatational axis such that the container holders counterbalance one another. The rotational mechanism is generally configured to rotate the container holder between about 0° and about 180°. In some embodiments, a controller is operably connected to the rotational mechanism. The controller typically comprises a logic device comprising logic instructions that direct the rotational mechanism to rotate the container holder at a selected rate. In addition, the cell culture dissociator also includes a stop that limits angular displacement of the container holder by the rotational mechanism. For example, impact forces from contacting the stop generally result in shear forces that are larger than attachment forces holding cells to container surfaces.

In some embodiments, the cell culture dissociator includes a container retention component (e.g., a container retention plate, etc.) that is movable relative to the container holder. The container retention component is structured to retain the cell culture container in a substantially fixed position relative to the container holder when the cell culture container is disposed in the container receiving area and the container retention component is in a closed position. Typically, the container holder and the container retention component are coupled to one another via at least one slidable coupling. Optionally, cell culture containers are retained in container holders with springs, with pneumatically driven levers, under an applied vacuum, etc. In certain embodiments, a controller is operably connected to the container holder. The controller generally includes a logic device comprising logic instructions that direct the container holder to close or open. Typically, the container retention component is structured to permit access to the cell culture container when the cell culture container is disposed in the container receiving area and the container holder is in the closed position.

In another aspect, the invention provides a dispensing device that includes a conduit that comprises an inlet and an outlet that fluidly communicate with one another, and a fluid source that fluidly communicates with the inlet of the conduit. Typically, a portion of the conduit that comprises the outlet is structured to fluidly communicate with a cell culture container. In some embodiments, for example, the portion of the conduit comprises a tip. Optionally, the tip comprises a ceramic coating and/or a non-coring profile. The dispensing device also includes a fluid conveyance device (e.g., a pump, etc.) operably connected to the conduit and/or to the fluid source. The fluid conveyance device is configured to convey at least a first fluid having a first selected temperature through the conduit from the fluid source. In addition, the dispensing device also includes a dispense head comprising at least one chamber through which at least a segment of the conduit passes, and a thermal regulation component that fluidly communicates with the chamber. Typically, the segment of the conduit is disposed proximal to the outlet of the conduit. In some embodiments, the dispense head comprises at least one manifold that fluidly communicates with the conduit. The segment of the conduit generally comprises a coiled structure. In some embodiments, a length of the conduit in the coiled structure is typically at least about 167 mm, although shorter lengths are also suitable. In certain embodiments, the dispensing device includes a plurality of conduits in which the dispense head comprises one or more segments of each of the conduits. In some of these embodiments, the dispensing device includes a plurality of fluid sources in which each of the conduits fluidly communicates with a different fluid source. The thermal regulation component is configured to flow at least a second fluid (e.g., an antifreeze solution, etc.) having a second selected temperature (e.g., about 37° C., etc.) into the chamber such that when the first fluid is flowed through the segment of the conduit the first fluid attains a temperature that is closer to the second selected temperature than to the first selected temperature. In some embodiments, when the first fluid is flowed through the segment of the conduit the first fluid substantially attains the second selected temperature. In certain embodiments, for example, the thermal regulation component comprises a fluid recirculation bath that substantially maintains the second fluid at the second selected temperature. Optionally, a fluid source storage device stores the fluid source at a first selected temperature (e.g., about 4° C., etc.).

In another aspect, the invention provides a method of dissociating cells in a cell culture container. The method includes (a) positioning a cell culture container that comprises a population of cells in a medium into a cell culture dissociator. The cell culture dissociator includes a container holder into which the cell culture container is positioned. The cell culture dissociator also includes a rotational mechanism operably connected to the container holder. The rotational mechanism can rotate the container holder about an axis. In addition, the cell culture dissociator also includes a stop that limits the angular displacement of the container holder by the rotational mechanism. Typically, the method includes dispensing at least one dissociative reagent (e.g., trypsin, etc.) into the cell culture container before, during, and/or after (a). In these embodiments, the cell culture container is generally incubated following the addition of the dissociative reagent and prior to being further processed. In some embodiments, (a) comprises (i) placing at least one cell culture container into a container receiving area of a container holder of the cell culture dissociator, and (ii) moving the container holder relative to a container retention component of the cell culture dissociator such that the cell culture container is retained in a substantially fixed position relative to the container retention component. The method also includes (b) rotating the cell culture container at an angular velocity that is sufficient to dissociate cells in the medium from one another and/or from one or more surfaces of the cell culture container when the stop is contacted. In certain embodiments, (b) comprises rotating a container holder of the cell culture dissociator into contact with one or more stops to produce shear force at least proximal to a surface of the cell culture container that comprises adherent cells. Optionally, the method includes (c) dispensing one or more materials into, and/or removing one or more materials from, the cell culture container while the cell culture container is positioned in the container holder.

In another aspect, the invention provides a method of passaging a cell culture. The method includes (a) placing a source cell culture container into a container holder of a cell culture dissociator in which the cell culture container comprises a population of cells and a liquid medium. In some embodiments, the method comprises performing a non-intrusive cell count of the cells in the source cell culture container prior to (a). Typically, the method includes dispensing at least one dissociative reagent (e.g., trypsin, etc.) into the source cell culture container before, during, and/or after (a). In these embodiments, the source cell culture container is typically incubated following the addition of the dissociative reagent and prior to being further processed. The method also includes (b) dissociating the cells from each other and/or from the cell culture container by using a rotational mechanism to: (i) rotate the container holder in a first direction until a first stop is contacted, which first stop limits the angular displacement of the container holder in the first direction, and (ii) rotate the container holder in a second direction, which second direction is opposite to the first direction, until a second stop is contacted, which second stop limits the angular displacement of the container holder in the second direction. Optionally, (b) further comprises repeating (i) and (ii) one or more times. In addition, the method also includes (c) transferring a portion of disassociated cells from the source cell culture container to each of one or more destination containers. The destination container is typically a daughter cell culture container and fresh cell culture media is added to the destination container. In certain embodiments, the method includes wetting the population of cells by rotating the source cell culture container with the rotational mechanism at a lower angular velocity than in (b). Optionally, the method includes removing at least some of the liquid medium from the source cell culture container before (b). In some embodiments, the method includes washing the population of cells in the source cell culture container before (b).

The method optionally includes expanding the cell culture by, in (c), transferring a portion of the disassociated cells into each of one or more destination containers. In certain embodiments, the source cell culture container is agitated (e.g., by a robotic arm, by the rotational mechanism, etc.) prior to (c) to obtain a uniform concentration of cells. In some embodiments, the method also includes (d) placing the destination containers in an incubation device. In some embodiments, the concentration of the dissociated cells is determined prior to (c). In these embodiments, a volume of the portion of the dissociated cells that is transferred to a destination container is calculated based on the cell concentration.

In some embodiments, the method further includes (d) pooling separate first cell culture media from m source cell culture containers in n destination containers to produce pooled cell culture media in which m is an integer greater than one, and n is an integer greater than zero and less than m. Typically, cells of the first cell culture media comprise a single cell line. In these embodiments, the method also typically includes (e) transferring selected volumes of the pooled cell culture media from the daughter cell culture containers into selected wells of p multi-well containers in which p is an integer greater than zero, thereby dispensing cell culture medium aliquots having substantially uniform cell concentrations.

In some embodiments, for example, n comprises an integer greater than one and (e) comprises transferring substantially equal volumes (e.g., about 5 mL, etc.) from at least one of the first cell culture containers into each of the second containers. Optionally, (d) comprises transferring volumes of cell culture media from at least one of the source cell culture containers to at least two of the destination cell culture containers. In certain embodiments, the source cell culture containers each comprise a volume capacity of about 10 mL, and/or the destination cell culture containers each comprise a volume capacity of about 100 mL. In some embodiments, (e) comprises transferring substantially identical volumes of the pooled cell culture media from the destination containers into substantially all wells of the multi-well containers. The multi-well containers each generally comprise, e.g., 6, 12, 24, 48, 96, 192, 384, 768, 1536, 3456, 9600, or more wells. Typically, the wells of at least one of the multi-well containers together comprise a volume capacity of about 10 mL. In some embodiments, m equals p, m equals an integer from 2 to 100 inclusive, p equals an integer from 2 to 100 inclusive, and/or a ratio of m:n is between about 1:1 and about 100:1 (e.g., about 5:1, about 10:1, about 25:1, about 50:1, about 75:1, etc.). Optionally, the method includes determining a concentration of cells in a pooled cell culture medium contained in at least one of the destination containers. As referred to herein, if cells are pooled following expansion using these methods, they are typically plated at uniform concentrations. If cells are not pooled according to these processes, they are optionally “harvested”. In some of these embodiments, for example, expanded cells are collected into large volume flasks (e.g., having volume capacities of between about 1 L and about 10 L) for use in other screening processes.

In another aspect, the invention provides a method of profiling one or more test reagents against a plurality of cell lines. The method includes, without human intervention, (i) dissociating cells of each of the plurality of cell lines, which cells are each contained in a cell culture container (ii) dispensing an aliquot of the cells of each cell line into one or more wells of a multi-well container, (iii) dispensing a test reagent into the well of the multi-well container, and (iv) detecting an effect of the test reagent on the cells. In some embodiments, upon completion of (ii), all cell-containing wells of a particular multi-well container contain cells of the same cell line. In other embodiments, upon completion of (ii), a particular multi-well container comprises wells that contain cells of a first cell line cell line and wells that contain cells of at least a second cell line. The test reagent typically comprises one or more reagents selected from, e.g., compounds, nucleic acids, proteins, viruses, bacteriophage, and the like. In some embodiments, fewer than 5,000 test reagents are profiled against each of the cell lines, whereas in other embodiments, 5,000 or more test reagents are profiled against each of the cell lines. The invention allows the automated profiling of test reagents against two or more, and in some embodiments at least 25, at least 50, or at least 100 cell lines. To illustrate, the effect of the test reagent on the cells is typically a stimulation or inhibition of one or more of cell proliferation, cell death, translocation, protein synthesis, etc.

In some embodiments, the dissociation of step (i) of the above method involves (a) positioning a cell culture container that comprises a population of cells in a medium into a cell culture dissociator that comprises: a container holder into which the cell culture container is positioned; a rotational mechanism operably connected to the container holder, which rotational mechanism can rotate the container holder about an axis; and a stop that limits the angular displacement of the container holder by the rotational mechanism; and, (b) rotating the cell culture container at an angular velocity that is sufficient to dissociate cells in the medium from one another and/or from one or more surfaces of the cell culture container when the stop is contacted.

### BRIEF DESCRIPTION OF THE DRAWINGS

FIG. 1 schematically illustrates a system that includes a single work perimeter according to one embodiment of the invention.

FIG. 2 schematically depicts a system that includes multiple work perimeters according to one embodiment of the invention.

FIG. 3A schematically shows a perspective view of a cell culture dissociator in which a container holder is in an open position relative to a container retention component according to one embodiment of the invention.

FIG. 3B schematically illustrates another perspective view of the cell culture dissociator shown in FIG. 3A in which the container holder is in a closed position.

FIG. 3C schematically depicts a perspective view of the cell culture dissociator shown in FIG. 3B in which the container holder is rotated about 90° relative to the position of the container holder shown in FIG. 3B.

FIG. 4A schematically shows a front elevational view of a cell culture passaging station according to one embodiment of the invention.

FIG. 4B schematically illustrates the cell culture passaging station of FIG. 4A from a side view.

FIG. 4C schematically depicts a portion of the cell culture passaging station of FIG. 4A from a perspective view.

FIG. 4D schematically depicts a portion of the cell culture passaging station of FIG. 4A from another perspective view.

FIG. 4E schematically depicts a portion of a material handling component of the cell culture passaging station of FIG. 4A from a perspective view.

FIG. 5 schematically shows a perspective view of a container positioning device according to one embodiment of the invention.

FIG. 6 schematically illustrates a front elevational view of a dispense head of a dispensing device according to one embodiment of the invention.

FIG. 7 schematically depicts a dispensing system that includes a thermal regulation component.

FIG. 8A schematically shows a cross-section through dispense head that includes a manifold according to one embodiment of the invention.

FIG. 8B schematically illustrates a cross-section through dispense head that includes a manifold according to another embodiment of the invention.

FIG. 9 schematically depicts one embodiment of a gripper apparatus from a side elevational view.

FIG. 10 schematically illustrates one embodiment of a grasping mechanism coupled to a boom of a robot from a perspective view.

FIG. 11A schematically illustrates another embodiment of a grasping mechanism coupled to a boom of a robot from a perspective view.

FIG. 11B schematically shows another exemplary embodiment of a grasping mechanism from a top perspective view.

FIG. 11C schematically depicts the grasping mechanism from FIG. 11B from a bottom perspective view.

FIG. 11D schematically shows a pivot member from a front elevational view according to one embodiment.

FIG. 11E schematically illustrates a pivot member from a front elevational view according to another embodiment.

FIG. 12 schematically shows a sample assaying component from a perspective view according to one embodiment of the invention.

FIG. 13A schematically depicts a pin tool from a perspective view according to one embodiment of the invention.

FIG. 13B schematically illustrates the pin tool from FIG. 13A from another perspective view.

FIG. 13C schematically shows the pin tool from FIG. 13A from an exploded perspective view.

FIG. 13D schematically illustrates a pin tool support structure and a top plate of a pin tool head from an exploded perspective view according to one embodiment of the invention.

FIG. 13E schematically shows a pin tool from a perspective view according to one embodiment of the invention.

FIG. 13F schematically depicts the pin tool from FIG. 13E from an exploded perspective view.

FIG. 13G schematically illustrates the pin tool from FIG. 13E from an exploded front view.

FIG. 13H schematically shows an interface between components of a pin tool head from the pin tool of FIG. 13E from a detailed front view.

FIG. 14A schematically shows a chassis of a fluid transfer device from a perspective view according to one embodiment of the invention.

FIG. 14B schematically depicts a pin tool attached to the chassis of FIG. 14A.

FIG. 15 schematically shows a sample assaying region from a perspective view according to one embodiment of the invention.

FIG. 16A schematically shows a support structure of a container positioning device from a top view.

FIG. 16B schematically depicts a cross-sectional side view of the support structure shown in FIG. 16A.

FIG. 16C schematically shows another cross-sectional side view of the support structure illustrated in FIG. 16A.

FIG. 16D schematically illustrates the support structure shown in FIG. 16A from a top perspective view.

FIG. 17A schematically shows a container positioning device that includes the support structure of FIG. 16A from a top view.

FIG. 17B schematically illustrates the container positioning device of FIG. 17A from a side elevational view.

FIG. 17C schematically illustrates the container positioning device of FIG. 17A from another side elevational view.

FIG. 17D schematically illustrates the container positioning device of FIG. 17A from a perspective view.

FIG. 17E schematically shows a perspective view of the positioning device of FIG. 17A mounted on a translational mechanism.

FIG. 17F schematically illustrates a sample assaying region from a perspective view according to one embodiment of the invention.

FIG. 17G schematically depicts a thermal modulation nest from a perspective view according to one embodiment of the invention.

FIG. 17H schematically shows the thermal modulation nest from FIG. 17G from a transparent top view.

FIG. 17I schematically shows a bottom plate of the thermal modulation nest from FIG. 17G from a top view.

FIG. 17J schematically illustrates the thermal modulation nest from FIG. 17G from a front view.

FIG. 17K schematically depicts the thermal modulation nest from FIG. 17G from a bottom view.

FIG. 18A schematically shows a container positioning device from a perspective view according to one embodiment of the invention.

FIG. 18B schematically shows the container positioning device of FIG. 18A from a partially exploded perspective view.

FIG. 18C schematically illustrates a partially transparent top view of a portion of a nest from the container positioning device of FIG. 18A.

FIG. 18D schematically shows the nest of FIG. 18C from a bottom perspective view.

FIG. 19 schematically illustrates fluid transfer probe vacuum drying station according to one embodiment of the invention.

FIG. 20A schematically shows a fluid transfer probe washing station from a perspective view according to one embodiment of the invention.

FIG. 20B schematically depicts another fluid transfer probe washing station from a perspective view according to one embodiment of the invention.

FIG. 21A schematically illustrates a wash reservoir that includes a transparent perspective view of a non-pressure-based fluid transfer probe mount according to one embodiment of the invention.

FIG. 21B schematically shows a non-pressure-based fluid transfer probe mounted on a non-pressure-based fluid transfer probe mount from a perspective view according to one embodiment of the invention.

FIG. 22 is a block diagram showing a representative fluid transfer probe washing station according to one embodiment of the invention.

FIG. 23A schematically shows a dispensing system from a perspective view according to one embodiment of the invention.

FIG. 23B schematically illustrates a detailed bottom perspective view of a dispensing component from the dispensing system of FIG. 23A.

FIG. 23C schematically depicts a detailed top perspective view of a dispensing component from the dispensing system of FIG. 23A.

FIG. 24 schematically shows a multi-channel peristaltic pump from a top perspective view.

FIG. 25 schematically depicts an object holder from a top perspective view.

FIG. 26A schematically shows a top view of a microtiter plate.

FIG. 26B schematically illustrates a bottom view of the microtiter plate shown in FIG. 26A.

FIG. 26C schematically depicts a cross-sectional view of the microtiter plate shown in FIG. 26A.

FIG. 27A schematically shows a partially transparent perspective view of a vacuum chamber of a cleaning component according to one embodiment of the invention.

FIG. 27B schematically illustrates a detailed cross-sectional view of a dispensing tip disposed proximal to an orifice of a portion of the vacuum chamber of FIG. 27A.

FIG. 28A schematically depicts a front cutaway view of one embodiment of an incubation device.

FIG. 28B schematically depicts a side cutaway view of the incubation device shown in FIG. 28A.

FIG. 29A schematically depicts a top cutaway view of one embodiment of an incubation device.

FIG. 29B schematically depicts a bottom cutaway view of the incubation device shown in FIG. 29A.

FIG. 30A schematically depicts a front view of one embodiment of an incubation device.

FIG. 30B schematically depicts a top view of the incubation device shown in FIG. 30A.

FIG. 31 schematically depicts a robotic gripping device interfacing with a door of an incubation device from a perspective view.

FIG. 32 schematically illustrates a modular object storage device and an a robotic gripping device from a perspective view.

FIG. 33 schematically depicts a perspective view of one embodiment of a fermentor.

FIG. 34 schematically illustrates a perspective view of one embodiment of an individual fermentation sample vessel.

FIG. 35 schematically shows one embodiment of an automated fermentation station.

FIG. 36 schematically illustrates a perspective view of an embodiment of an automated centrifuge.

FIG. 37 schematically shows a perspective view of a section of a rotor employed in the centrifuge illustrated in FIG. 36.

FIG. 38 schematically illustrates a plan view of the rotor illustrated in FIG. 37.

FIG. 39 schematically illustrates a perspective view of a transport and waste trough illustrated in FIG. 36.

FIG. 40 schematically shows a perspective view of the waste trough illustrated in FIG. 39.

FIG. 41 schematically illustrates a perspective view of a sample/fraction collector illustrated in FIG. 36.

FIG. 42 is a block diagram illustrating a method of performing a compound profiling assay according to one embodiment of the invention.

FIGS. 43A-C schematically illustrate methods of transferring substantially uniform concentrations of cells from cell culture flasks into multi-well plates according to certain embodiments of the invention.

FIG. 44 schematically illustrates a representative compound profiling system in which various aspects of the present invention may be embodied.

FIG. 45 schematically shows another representative compound profiling system in which various aspects of the present invention may be embodied.

FIG. 46A is a flow chart illustrating aspects of control software architecture according to specific embodiments of the invention.

FIG. 46B shows a display screen related to the control software architecture depicted in FIG. 46A according to one embodiment of the present invention.

FIG. 47A is a flow chart illustrating aspects of control software architecture according to specific embodiments of the invention.

FIG. 47B schematically shows an interface of the control software depicted in FIG. 47A according to one embodiment of the invention.

FIG. 47C show display screens for submitting requests that are related to the control software architecture depicted in FIG. 47A according to one embodiment of the present invention.

FIG. 47D shows a display screen for monitoring requests (report view) that are related to the control software architecture depicted in FIG. 47A according to one embodiment of the present invention.

FIG. 47E show display screens depicting various exemplary operator tools that are related to the control software architecture depicted in FIG. 47A according to some embodiments of the present invention.

FIG. 47F shows a diagram that depicts certain software component interfaces with other system software components that are related to the control software architecture depicted in FIG. 47A according to one embodiment of the present invention.

FIGS. 48 A and B are flow charts illustrating exemplary scheduler software protocols according to specific embodiments of the invention.

FIG. 49 is a schematic cross-section of a conduit for use in some embodiments of the present invention.

FIG. 50A is a perspective view demonstrating the use of a single-well plate with a detector.

FIG. 50B is a schematic cross-section of the single-well plate of FIG. 50A.

FIG. 51 is a perspective view demonstrating the dispensing of fluid directly from a flask, via an eight-way manifold.

### DETAILED DESCRIPTION OF THE PREFERRED EMBODIMENT

### I. Definitions

Before describing the present invention in detail, it is to be understood that this invention is not limited to particular embodiments. It is also to be understood that the terminology used herein is for the purpose of describing particular embodiments only, and is not intended to be limiting. As used in this specification and the appended claims, the singular forms “a,” “an,” and “the” also include plural referents unless the context clearly provides otherwise. Thus, for example, reference to “a container holder” also includes more than one container holder. Units, prefixes, and symbols are denoted in the forms suggested by the International System of Units (SI), unless specified otherwise. Numeric ranges are inclusive of the numbers defining the range. Further, unless defined otherwise, all technical and scientific terms used herein have the same meaning as commonly understood by one of ordinary skill in the art to which the invention pertains. The terms defined below, and grammatical variants thereof, are more fully defined by reference to the specification in its entirety.

The term “adherent cells” refers to cells that are bound, stuck, connected, or otherwise associated with one another and/or with another object, such as a surface of a cell culture flask or other container.

The term “angular displacement” refers to an angle that a rotating body rotates through. In some embodiments, for example, a rotation mechanism of a cell culture dissociator rotates cell culture containers disposed in a container holder of the dissociator through a selected angle as part of a process to dissociate cells in the containers.

The term “angular velocity” refers to a rate of rotation around an axis. Angular velocity is typically expressed in radians or revolutions per second or per minute. In some embodiments, for example, a cell culture container is rotated at an angular velocity that is sufficient to dissociate cells disposed in the container from one another and/or from one or more surfaces of the container.

The term “automated” refers to a process, device, sub-system, or system that is controlled at least in part by mechanical and/or electronic devices in lieu of direct human control. In certain embodiments, for example, the compound profiling systems of the invention include automated cell culture passaging stations that are configured to sub-culture or split cell cultures in the absence of direct human control. In some embodiments, all components or devices of the compound profiling systems of the invention are automated.

Device or systems components “communicate” with one another when fluids, energy, pressure, information, objects, or other matter can be transferred between those components. To illustrate, fluid sources fluidly communicate with the inlets of conduits such that fluids can be flowed or otherwise conveyed through the conduits in some embodiments. To further illustrate, thermal regulation components thermally communicate with conduits in certain embodiments so that thermal energy can be transferred between the thermal regulation components and the conduits to regulate or control the temperatures of fluidic reagents conveyed through the conduits.

The term “counters” in the context of rotational mechanisms of cell culture dissociators refers to the act of one object in opposition to or otherwise offsetting another object. In some embodiments, for example, a rotational mechanism includes a counterweight that offsets the weight of a container holder and/or the weight of a cell culture container disposed in the container holder.

The term “fluidic material”, “fluidic reagent”, or “fluid” refers to matter in the form of gases, liquids, semi-liquids, pastes, or combinations of these physical states. Exemplary fluids include certain reagents for performing a given assay, various types of media for supporting a cell culturing process, suspensions of cells, beads, or other particles, and/or the like.

The term “non-coring profile” in the context of fluid handling tip profiles refers to a profile that permits the tip to be insert into or through an object and/or removed from that object substantially without removing any material from the object. In some embodiments, for example, a tip having a substantially smooth outer surface and a tapered profile at least proximal to the end of the tip is inserted through an elastomeric septum that seals a container and is removed from the septum during a given fluid handling process substantially without removing any elastomeric material from the septum.

The term “shear force” refers to a force that is directed substantially tangential to the section of the object on which it acts. To illustrate, a container holder of a cell culture dissociator is rotated into contact with one or more stops in some embodiments to produce a force that acts tangential to a surface of a cell culture container disposed in the container holder to dissociate cells adhered to that surface.

The term “substantially” refers to an approximation. In certain embodiments, for example, a container retention component of a cell culture dissociator retains a cell culture container in a fixed or approximately fixed position when the container holder of the device is in a closed position. To further illustrate, a thermal regulation component thermally communicates with the conduits of a dispensing device in some embodiments so that when fluidic reagents are flowed through the conduits, the fluidic reagents attain or approximately attain a selected temperature.

The term “translational axis” refers to one of the three linear axes (i.e., X-, Y-, and Z-axes) in a three-dimensional rectangular coordinate system.

The term “work perimeter” refers to an area within the reach of a robotic device. In some embodiments, for example, the work perimeter of a rotational robotic gripping device is the area with the rotational reach of the device.

### II. Introduction

While the present invention will be described with reference to a few specific embodiments, the description is illustrative of the invention and is not to be construed as limiting the invention. As will be apparent to those skilled in the art, various modifications can be made to certain embodiments of the invention without departing from the true scope of the invention as defined by the appended claims. It is noted here that for a better understanding, like components are designated by like reference letters and/or numerals throughout the various figures.

The present invention provides flexible, robust, accurate, and reliable systems and methods that can be used in performing various high throughput processes, including, e.g., profiling samples (e.g., test compounds or reagents, siRNAs, cDNAs, viral particles, bacteriophage, proteins, antibodies, as well as other screenable factors or perturbagens) against multiple assays as part of compound profiling applications (e.g., involving single or multiple cell lines, etc.). The invention alleviates to a great extent the disadvantages of known systems and methods for screening, analysis, and assembly. For example, the systems described herein provide linear or multi-directional and non-linear transport between multiple devices. Accordingly, the systems and methods of the invention improve the reliability, efficiency, and flexibility of processes, such as high throughput screening and other methods that utilize repetitive manipulations of many individual elements.

A typical system of the invention comprises one or more rotational robots (e.g., robotic gripping devices, etc.) that are each associated with a work perimeter. Each work perimeter typically includes one or more devices or sub-systems, e.g., in various station locations within the work perimeter. In addition, each station location and/or device is generally configured to be accessible by the robot associated with the work perimeter in which the device is positioned. Typically, at least one work perimeter has at least two devices that are exclusively within the reach of the associated rotational robot.

In embodiments that include multiple work perimeters, transfer stations are also generally included, e.g., disposed between adjacent work perimeters, to facilitate the transfer of objects, such as sample containers from one work perimeter to another work perimeter. In addition, the overall system is typically coupled to a controller that includes a PC or other logic device, e.g., for directing the transport of sample containers between work perimeters and for directing sample processing by devices in those work perimeters. The controllers are typically configured to receive operator instructions and to provide operator information.

The systems of the invention provide flexibility in multiple ways. To illustrate, the devices used in the systems of the invention are optionally arranged and positioned at selected station locations according to the specific requirements of a desired application. Therefore, the entire system is optionally tailored to a specific application. In addition, the systems offer flexibility within each application. For example, the devices in the system are optionally accessed in any order. The controller is optionally programmed to access the station locations in any order, including backtracking to a previously used assaying device. The random access and random processing provided by the systems described herein increase process throughput relative to pre-existing systems, e.g., since the throughput of these systems is not limited to the speed of the particular robot being utilized.

Additional advantages provided by the invention include that each robot in a given system efficiently transfers objects between all devices within that robot's work perimeter. This close association between a robot and the devices or sub-systems within its work perimeter facilitates increased processing throughput, reliability, and accuracy. In addition, since devices and/or station locations are easily added, removed, or reconfigured, the systems are highly flexible or adaptable. Since work perimeters generally include a plurality of station locations and/or devices, the overall system generally needs relatively few work perimeters and associated robots to perform a given automated process, such as compound profiling. Thus, the transport sample containers or other objects between system components can be efficiently and rapidly accomplished. Moreover, the invention provides for multi-directional transporting within these systems. Processing optionally occurs in any order and is independent of the physical configuration of the station locations. A system made in accordance with the present invention performs high throughput processing quickly, accurately, and with great flexibility, as described in more detail below.

To further illustrate, a robot in a first work perimeter is optionally used to transport a sample container from a container storage device, e.g., located in a first work perimeter, to a transfer station, from which transfer station the sample container is retrieved by a second robot and transported, e.g., to a second work perimeter that includes an assaying device or component, an automated cell culture passaging station, or other device. Alternatively, aliquots of samples in the sample container can be transferred at the transfer station to a different sample container such as, an assay sample container. In the second work perimeter, the sample container is optionally processed, e.g., by transporting the sample container to an assaying component for assaying the samples. The processing steps are also flexible, in that a sample is optionally assayed, detected, and then assayed again, e.g., using a second assaying device or by transporting the sample container back to the first assay device. The samples are thus optionally allowed to proceed, e.g., from an assaying step, to a dispensing or detecting step, and back to the assaying step, e.g., as directed by a controller, without having to rearrange the entire system or having an operator manually transport the samples. This flexibility decreases the need for system reconfiguration, e.g., by moving various devices around, thereby also decreasing the risk of contamination, e.g., by decreasing the need to handle the sample containers.

The samples processed by the systems of the invention are typically contained in one or more sample containers, e.g., microwell plates, such as 96, 384, or 1536-well plates. Such samples include, but are not limited to, chemicals, biochemicals, serum, cells, cell extracts, nucleic acids (e.g., cDNAs, RNAs, etc.), viruses, bacteriophage, proteins, enzymes, antibodies, carbohydrates, lipids, blood, inorganic materials, and the like.

The systems of the invention are optionally used for high throughput screening of samples, e.g., of chemical compounds against, for example, cells, cell extracts, and/or particular molecular targets as part of compound profiling processes. Accordingly, the systems and methods described herein can be used to identify novel, bioactive compounds that modulate biological processes and to identify cellular and molecular targets, e.g., of small molecules.

Chemical compounds identified by high or ultra-high throughput screening are optionally used as tools for probing and profiling cellular responses and the key molecular entities underlying them. In addition, chemical compounds identified using the systems and methods of the invention are optionally used as lead compounds for therapeutic, prognostic and diagnostic applications. As one example, the systems and methods described herein can be used to perform efficient, comprehensive, functional pathway scans on intact cells, thereby screening, e.g., fewer than 5,000 compounds, or in some embodiments up to about 100,000 or more putative perturbagens or other compounds per day in a 1536-well format. In some embodiments, the cell-based, biochemical, or other screening systems of the invention screen about 10,000, 50,000, 100,000, 250,000, 500,000, 750,000, or more samples in about 1 to about 4 days with high reliability. The large capacity of these systems also typically provides reduced costs, e.g., on a per assay basis.

To further summarize, the present invention provides processing systems that are not limited by robot speed or to rectilinear sequential access to devices. These systems provide random access to and multidirectional transport between multiple devices. In addition, these systems provide reliable and accurate processing of, e.g., large numbers of samples in a high or ultra-high throughput manner (e.g., using 1536-well or higher density plates in some embodiments). The systems of invention are also very flexible, being able to incorporate various sub-systems or devices as needed for a particular application. In some embodiments, for example, the compound profiling systems of the invention include sub-systems that perform various functions including, e.g., automated storage and retrieval of cell lines or cultures, automated passaging of stored cell lines (including the scheduled collection of aliquots for freezing to preserve the cell lines), automated expansion of selected cell cultures for profiling assays (including periodic cell counting to adapt the processing parameters for optimum expansion and so that all cell lines in a group are grown up at the same time), automated concentrating and plating, automated assay performance, control systems, and data management. Exemplary components of these systems are discussed in detail below, followed by example systems and methods of using them.

### III. Systems and System Components

The present invention provides processing systems that are useful, for example, for profiling one or more target molecules and/or test compounds or reagents against two or more assays. The systems typically provide an automated robotic process for handling, mixing, moving, storing, assaying, and detecting samples. For example, the systems are optionally designed to carry out assaying, measuring, dispensing, and detecting steps, e.g., in multi-well plates.

Typically, the systems include at least one work perimeter and at least one robotic gripping device (e.g., a rotational robotic gripping device, etc.), e.g., from about one to about 10 work perimeters and/or robots. Each rotational robot is typically associated with one or more of the work perimeters. The robots each typically have a reach that defines the work perimeter associated with that robot. The work perimeters and robots are generally configured to allow the transport of sample containers along a multi-directional path, e.g., to provide a flexible transport system for a plurality of sample containers. In addition, the systems generally include at least one device or sub-system associated with each work perimeter. In some embodiments, a work perimeter includes two or more devices within the reach of an associated rotational robot. The systems are optionally configured to provide sequential or non-sequential transport between the two or more devices, with each device being accessible by at least one of the rotational robots. To further aid the transport of multiple sample containers, the systems typically include one or more transfer stations disposed between adjacent work perimeters. The transfer stations provide sample transfer points between work perimeters (e.g., by providing for the transfer of the containers themselves or the transfer of aliquots of samples from one sample container to another). Work perimeters and transfer stations are described further below.

As referred to above, each work perimeter in the systems of the invention generally contains one or more devices or sub-systems for performing selected functions. These devices are typically automated instruments that are used to, e.g., store, dispense, measure, assay, detect, or otherwise process fluids, reagents, samples, etc., e.g., in sample containers. The devices are generally located in or on a station location, e.g., a platform or table comprising electrical connections and computer and/or controller connections. The devices are typically positioned at a station location prior to operation of the system, however, a device is optionally added to a station location during operation of the system as well. In addition, the devices are optionally moved around within a work perimeter, e.g., either before an operation of the device or upon reconfiguration prior to using the device for another application. The devices need only being positioned within a work perimeter, e.g., to be within the reach of the rotational robot associated with the work perimeter. If enough station locations are not available, a device is optionally positioned within the reach of the robot without a dedicated station location. In some embodiments, at least two devices within at least one work perimeter are exclusively within the reach of the associated robot.

Typically each station location in the system contains a single device, however, multiple devices are optionally positioned at a single location as well. In addition, the system may comprise station locations that do not have associated devices or devices that are not associated with a station location. Unoccupied station locations are optionally used for storage, temporary holding of sample holders, or simply not accessed during operation of the system. In addition, all devices are not necessarily used during operation of the system. A number of devices are typically positioned within the station locations of the system prior to operation. During operation of the system, all of the devices are optionally used or only a portion of the devices may be used. Because the rotational robots access each station location independently, the devices are accessed in any order desired, including skipping some devices all together and/or repeatedly accessing one or two devices. An operator typically programs the system, e.g., via a controller, to transport the sample holders from device to device as desired for a particular application.

In addition, the devices typically each have a receiving module, e.g., for receiving a sample container. In some cases, the receiving module couples to a gripper or positioning device on a robotic arm. In some devices, sample containers are placed on a conveyor by the robotic arm or placed in a sample compartment or positioning device. For example, the robotic arm optionally opens a door on an incubator and places the sample container inside the incubator on, e.g., a shelf of a hotel or carousel.

The devices used in the systems of the invention include, but are not limited to, compound storage devices or modules, liquid dispensers, workstations, replating stations, thermocyclers, incubators, heating units, pumps, detectors, electrophoresis and/or chromatography modules, purification and/or filtration modules, wash modules, centrifuges, PCR modules, vacuums, refrigeration or freezer units, mixing plates, weighing modules, light sources, and other types of devices known to those of skill in the art. Such devices are used to perform a variety of processes including, but not limited to, PCR, hybridizations, cloning, hitpicking, translation, transcription, isolations, cell growth, washes, dilutions, detection, and the like. Some of these exemplary devices are described further below.

A. Work Perimeters, Station Locations, and Transfer Stations

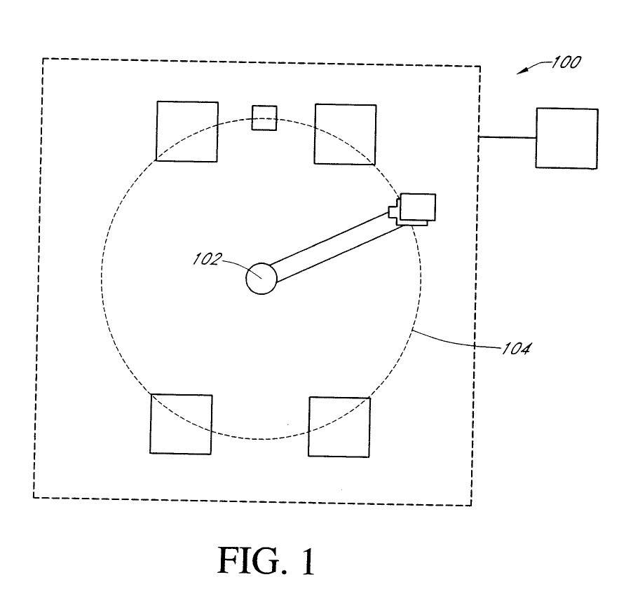

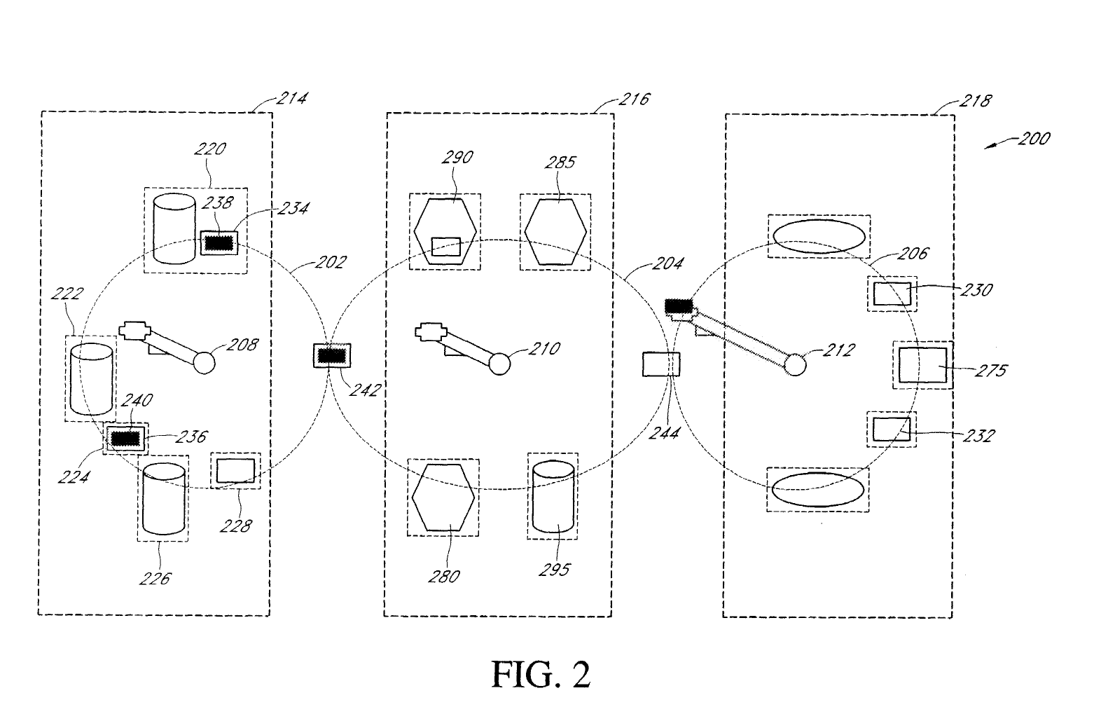

The work perimeters of the systems of the present invention typically comprise one or more station locations, and often two or more station locations. The station locations are used to perform various processes, assays, and the like, e.g., on the samples within a sample plate or holder. The reach of a robot (e.g., a rotational robot, etc.) generally defines its associated work perimeter. For example, FIG. 1 schematically depicts system 100 that includes rotational robot 102, the reach of which defines work perimeter 104. To further illustrate, FIG. 2 schematically depicts system 200 that comprises three work perimeters 202, 204, and 206. As shown, the work perimeters 202, 204, and 206 comprise areas in which devices and stations are placed and are defined respectively by the rotational reach of robots 208, 210, and 212. The rotational reach areas are shown as circles or ovals but are optionally any other shape, depending on the reach and extension of the robot arm. Typically, at least one work perimeter has two or more devices exclusively within the reach of the rotational robot within that work perimeter. In some embodiments, two or more work perimeters have two or more devices exclusively within the reach of the rotational robot within each particular work perimeter. In the specific embodiments shown in FIGS. 1 and 2, each depicted work perimeter has two or more devices exclusively within the reach of the respective robot. Optionally, processing systems include one or more work perimeters having only a single device exclusively within the reach of the rotational robot within those work perimeters.

Although FIG. 1 illustrates one work perimeter and FIG. 2 shows three work perimeters, the number of work perimeters is optionally two or more than three, depending on specific assay requirements. Typically a work perimeter is provided for each rotational robot in use and the work perimeter extends at least as far as the rotational reach of the robot. The devices associated with each work perimeter can encompass additional space, for example, as shown in work areas 214, 216, and 218 in FIG. 2. The rotational robot needs to reach only far enough to place a sample or sample container in or on the desired device. For example, a dispensing device optionally uses up space beyond the rotational reach of an associated robot, e.g., to accommodate a pump and or a waste receptacle, yet the robot optionally reaches only far enough for the dispensing device to receive the sample holder.

Each work perimeter is optionally directed to a certain task or group of tasks, e.g., using the station locations and devices located within that area. For example, a first work perimeter is optionally used for storing samples or compounds, while a second work perimeter is used for processing a sample or group of samples, e.g., by adding reagents, shaking, heating, incubating, or the like. A third work perimeter is optionally used for analyzing and/or detecting the samples once they have been assayed. Further, a sample is optionally separated into various components, which are then detected, e.g., using a fluorescent detector. Alternatively, each work perimeter is directed to a particular type of assay in a process that involves multiple assays. Although each work perimeter is generally directed to a particular type of task, e.g., detection, storage, or the like, the functionality of the work perimeters is optionally overlapping. For example, a work perimeter that is generally used for storage, may also be used to perform a heating or incubation step in an assay of interest or some other processing step.

One advantage of the systems of the present invention is that there is no particular order that must be followed in transporting samples between work perimeters, as is the case with many pre-existing systems. Because the systems described herein have multidirectional utility, samples are optionally transported from the first work perimeter to the second work perimeter and then back to the first area, e.g., for further processing, prior to detection in a third work perimeter. This provides an operator the ability to respond, e.g., to results or information gathered in a first assay, and re-program the system accordingly for further processing, e.g., further dilution in a different work perimeter can be directed during operation if a sample is found to be too concentrated in a detection step.

In addition, as each work perimeter generally accommodates a plurality of devices, and work perimeters are positioned adjacent each other, an entire high throughput screening system is optionally configured to fit into a reasonably compact physical space. For example, system 200 as shown in FIG. 2 can fit in an 18′×12′ space. Fitting into a compact space not only is efficient from a cost standpoint, but also facilitates efficient movement of sample holders between work perimeters and from one end of the system to the other end of the system. By enabling a compact physical arrangement, the speed and efficiency of the overall system are increased. Further, because peripheral devices are compacted into a small physical area, the amount of time a specimen plate is in transport, and potentially uncovered, is reduced. Thus, the risks of contamination and undesirable evaporative effects are reduced. Another advantage resulting from the compactness of the systems of the present invention is the ability to enclose entire systems in one or more chambers having sterile or otherwise controlled environments. As such, environmental effects such as temperature, pressure, humidity, and particle content can be strictly maintained.

Station locations are areas that are used to accommodate one or more devices or sub-systems, or sample containers. The station location is typically a place, e.g., a table, platform, or location that is configured to receive a device, e.g., a cell culture passaging station, a fluid dispenser, a plate carousel, a detector, or the like. Each work perimeter of the systems of the invention typically comprises two or more station locations. For example, FIG. 2 illustrates various station locations, e.g., station locations 220, 222, 224, 226, and 228 in work area 214. Each work perimeter typically comprises two or more station locations that optionally include one or more devices.

Typically, each station location comprises one device for a given assay or process, e.g., a thermocycler, a pump, a fluid dispenser, an incubator, a storage module, or the like. The devices will typically remain at a single station location during an entire process and be accessed, e.g., in any order desired, by the rotational robots.

Alternatively, the station locations are adapted to a particular process before operation of the system, such that every station location comprises a device of use in the immediate process. In this manner, the station locations convey a great deal of flexibility to the system. Each location is typically set up or configured to receive a device. For example, a controller is optionally associated with each station location, e.g., for sending and receiving process information. In addition, electrical connections are typically provided for each station location, such that whenever a new device is desired, the hook up at a station location is easily accomplished. In addition, because the station locations are not necessarily located along a linear path (e.g., a conveyor or the like), device alignment problems are typically decreased relative to those encountered in many pre-existing systems.

In some embodiments, one or more station locations are empty or unused in a given process. For example, a station location optionally is left empty or used as a holding area, as described below. In addition, some station locations have devices positioned therein that are not used in a particular process. In that case, the rotational robots are not instructed to transport the sample containers to that station location, which is skipped in the transport path selected. No time is wasted by having to transport the sample holders through an unused station. Therefore, the system provides improved throughput and efficiency.

In some embodiments, the station locations comprise platforms, e.g., platforms that are optionally raised and lowered, e.g., mechanically, hydraulically, pneumatically, etc. In other embodiments, the station location is merely a designated place on a table or bench to which a device is optionally affixed. The station locations act as place holders for devices and are optionally any shape and size depending on the devices of interest.

Although system 200, shown in FIG. 2, only defines a select number of station locations, more or fewer station locations are optionally defined depending upon the reach of each robotic arm and the size of the selected devices. Further, station locations are optionally added, moved, or removed depending on specific application needs. For example, a given work perimeter optionally includes about 2 to about 10 station locations, and more typically about 3 to about 5 station locations.

Because station locations can remain the same irrespective of what device is positioned in that station location, systems are easily reconfigured to accommodate a variety of specific needs. Accordingly, systems (e.g., high throughput compound screening or profiling systems) of the invention are optionally reconfigured to add, delete, or replace devices in any station location. Moreover, station locations are also optionally added or removed to accommodate changes in the area or robot orientation. Thus, not only are these systems readily reconfigured, they are also easily adjusted to accommodate adjustments in work flow.

In addition to station locations, work perimeters also optionally comprise holding areas, e.g., for temporarily storing sample containers until needed in a particular assay. As shown in FIG. 2, for example, system 200 includes holding areas 230 and 232 in work area 218 and holding areas 234 and 236 in work area 214. Holding areas 234 and 236 in FIG. 2 are shown with sample containers 238 and 240 positioned respectively therein. These holding areas optionally contain positioning devices or nest devices, such as static exchange nests or interchange platforms. In one embodiment, an operator uses one or more of static holding areas, e.g., to manually introduce sample plates into a system. Any number of temporary holding areas is optionally used in the high throughput screening systems of the invention. In fact, the number of holding areas is variable within the same system and is optionally changed from one operation to the next.

In system 200 illustrated in FIG. 2, holding areas 230, 232, and 236 are positioned away from any instrumentation and provide temporary resting areas, e.g., for sample containers. To illustrate, timing considerations sometimes dictate that a sample container should rest for a period of time, e.g., at a holding area. In addition, the holding areas are optionally used to carry out one or more processes. For example, filtration of samples, application of vacuum pressure, or UV exposure of the samples in the sample holder, are optionally carried out in a holding area. Also, a holding area optionally accommodates the temporary holding of a sample container when the next sequential device is not yet available. The robot typically retrieves the sample container from the temporary holding area and moves it to the next sequential device when that device is available.

A transfer station (or hand-off area) is typically a location located proximal to two or more work perimeters, e.g., for transferring containers (e.g., multi-well containers, cell culture flasks, etc.) between work perimeters. In some embodiments, transfer stations include platforms that are used for placing the container, e.g., until an adjacent rotational robot retrieves it. However, transfer stations are also optionally areas, e.g., on a system surface or a table surface, in which two or more robotic arms meet and transfer a container or other object directly from one arm to the other.

In addition to transferring containers from one device to a second device or from one work perimeter to another work perimeter, transfer stations are also optionally used to transfer samples from one container to another container, e.g., in a replating procedure as described in more detail below. Typically, a container, e.g., containing test reagents for screening, is transferred from a container storage device to a transfer station. From the transfer station, containers (e.g., compound plates, etc.) can be transferred to an adjacent work perimeter. Either the entire container can be transferred to a particular work perimeter, or sample aliquots from the container can be transferred to an assay plate. For example, a robot in one work perimeter optionally transfers an assay plate to a transfer station that includes a fluid transfer device, which transfers aliquots of test reagents from reagent plates into the wells of the assay plate. The reagent plate is then put back into the storage device, and the assay plate is subjected to further processing (e.g., addition of additional reagents, incubation, mixing, etc.). Typically, after a desired incubation time or immediately after a further processing step, the assay plates are moved to a detection component of a system. Exemplary transfer stations are schematically depicted in, e.g., FIG. 2 (transfer stations 242 and 244).

Work perimeters and related system configurations that are optionally adapted for use with the systems of the present invention are also described in, e.g., U.S. Patent Publication No. 2002/0090320, entitled “HIGH THROUGHPUT PROCESSING SYSTEM AND METHOD OF USING,” filed Oct. 15, 2001 by Burow et al., which is incorporated by reference.

B. Cell Culture Passaging Stations

The systems of the invention typically include cell culture passaging stations that passage (i.e., split or sub-culture) cell cultures. These stations are generally fully automated such that cell culture libraries can be automatically passaged according to user-defined schedules. Typically, these cell culture libraries include two or more, and in some cases 25 or more, or even hundreds or thousands of cell cultures disposed in various types of cell culture containers (e.g., cell culture flasks, etc.). In certain embodiments, these passaging stations are also configured, e.g., to effect the monitoring of cell health and density status and/or the automatic archiving of cell culture sample aliquots from particular cell culture containers by freezing or otherwise preserving those sample aliquots according to a selected schedule. Exemplary automated cell culture passaging processes using these stations are also described below in an example.

The automated cell culture passaging stations of the invention generally include cell culture dissociators and material handling components. Cell culture dissociators are typically configured to effect cell wetting, dissociation, and/or agitation functions, while material handling components are generally configured to effect the transfer of material (e.g., cell culture media, reagents, etc.) to and/or from cell culture and other containers. Other exemplary components that are optionally included in the cell culture passaging stations of the invention include container positioning devices, decontamination devices, and translational mechanisms. Cell culture passaging station components are also typically operably connected to suitable controllers that are configured to effect their operation. Each of these cell culture passaging station components is described further below. It will be appreciated that these components can also be adapted for use in other devices or sub-systems, including those of the compound profiling systems described herein.

i. Cell Culture Dissociators

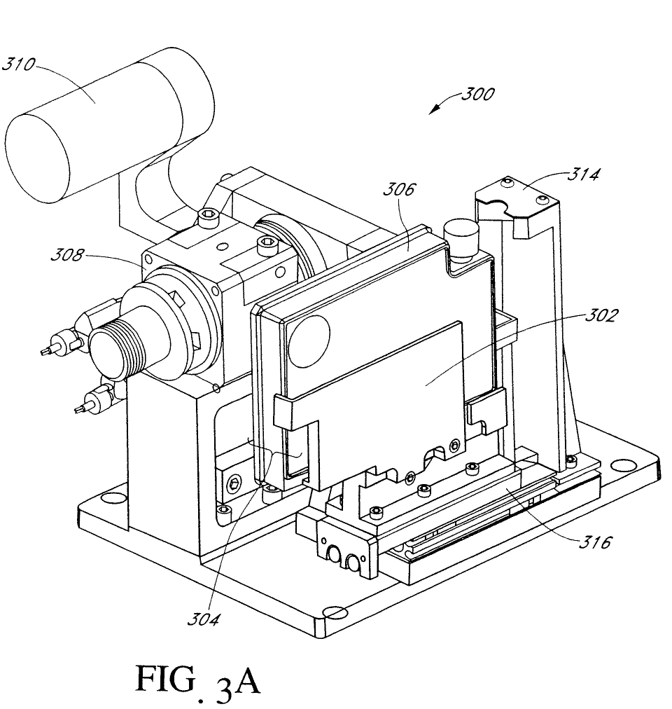

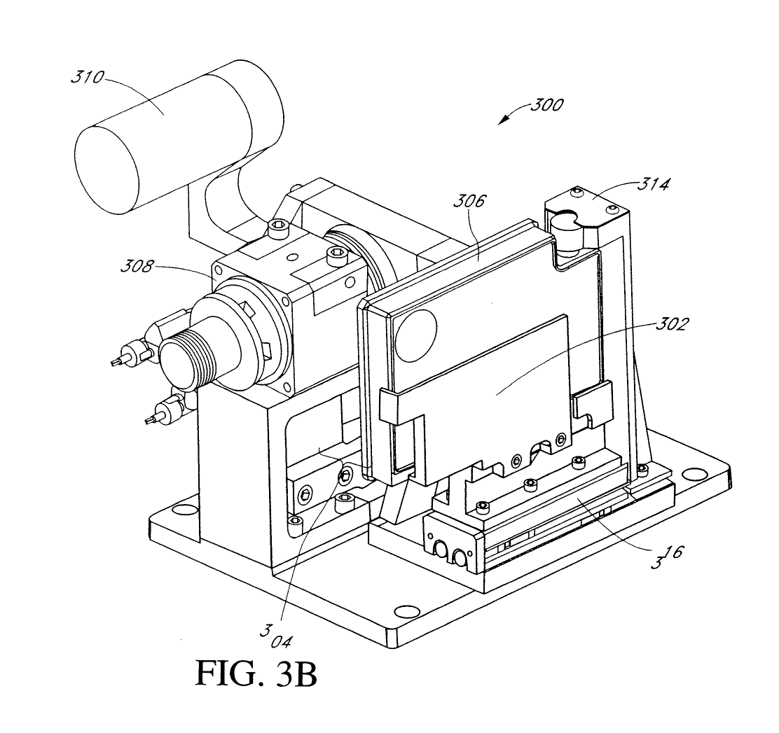

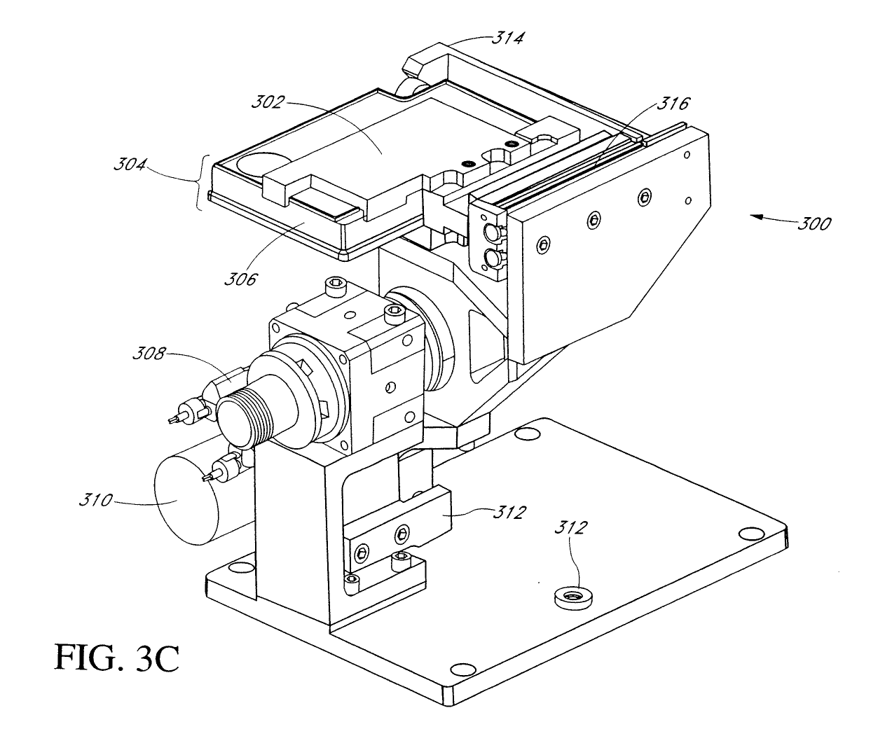

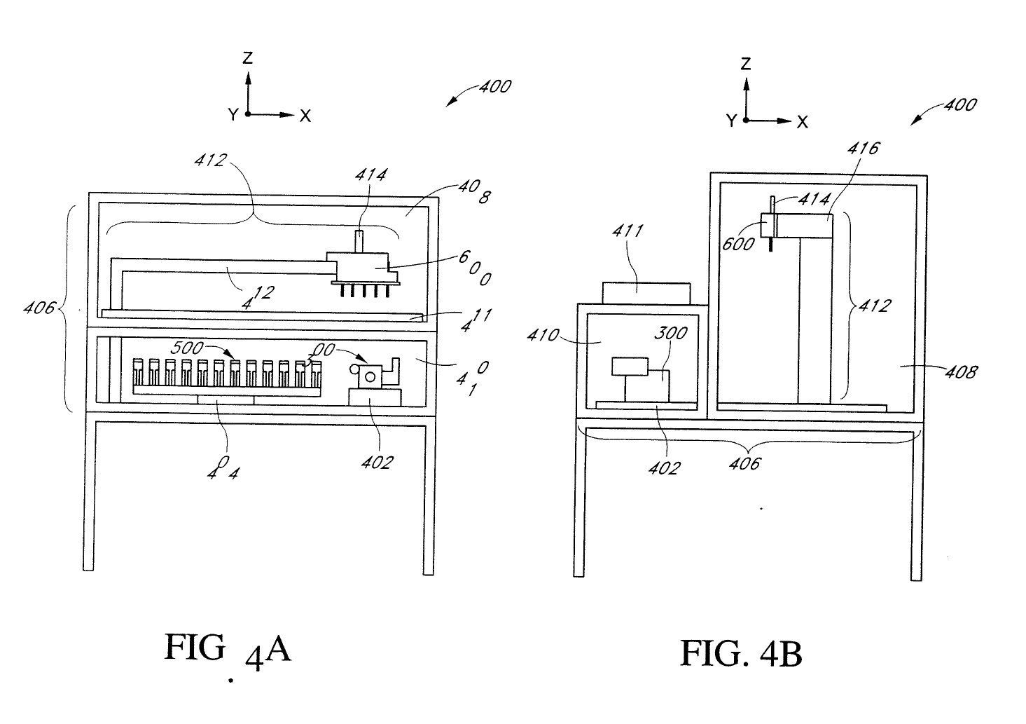

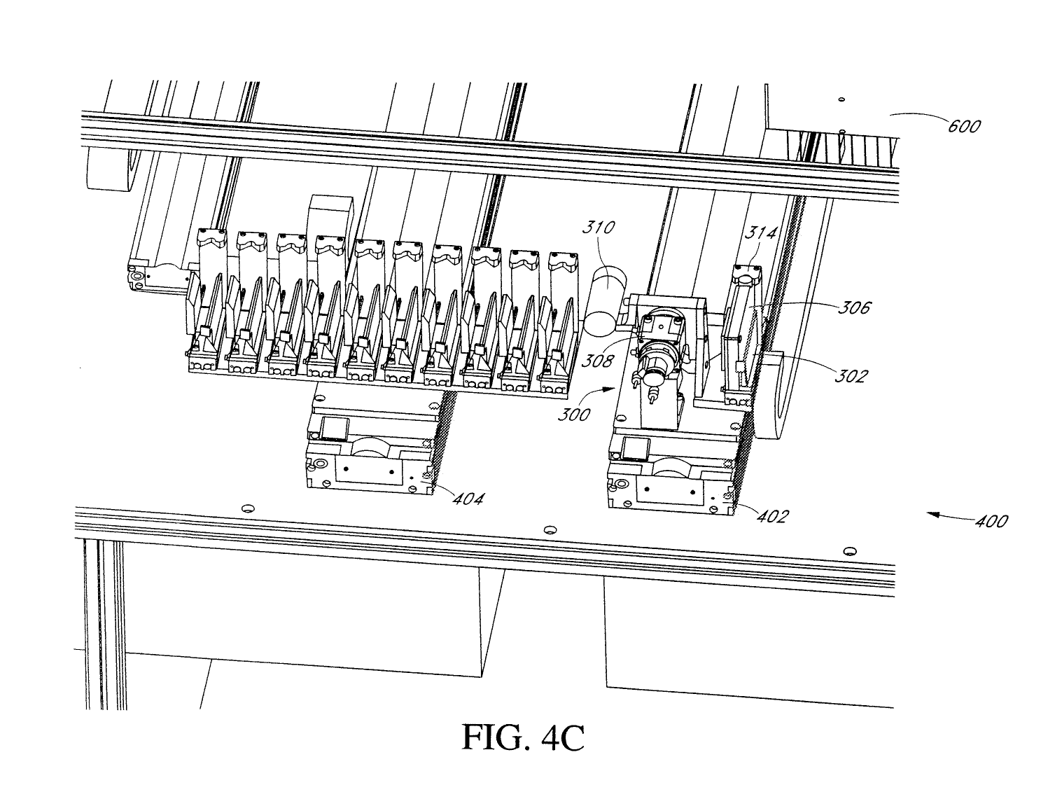

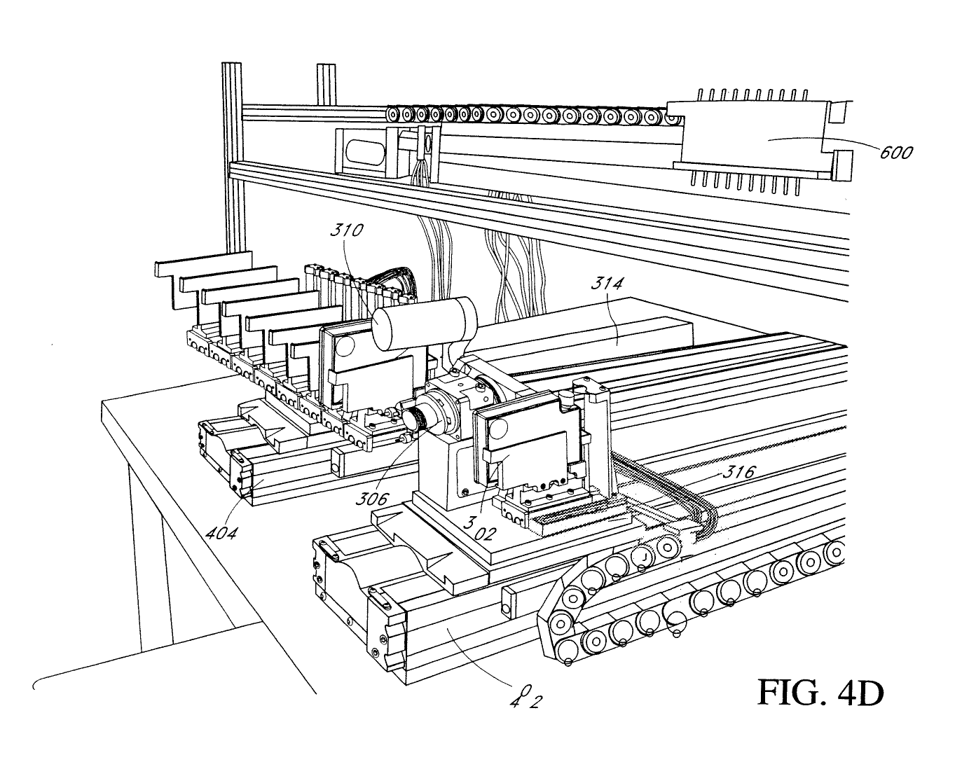

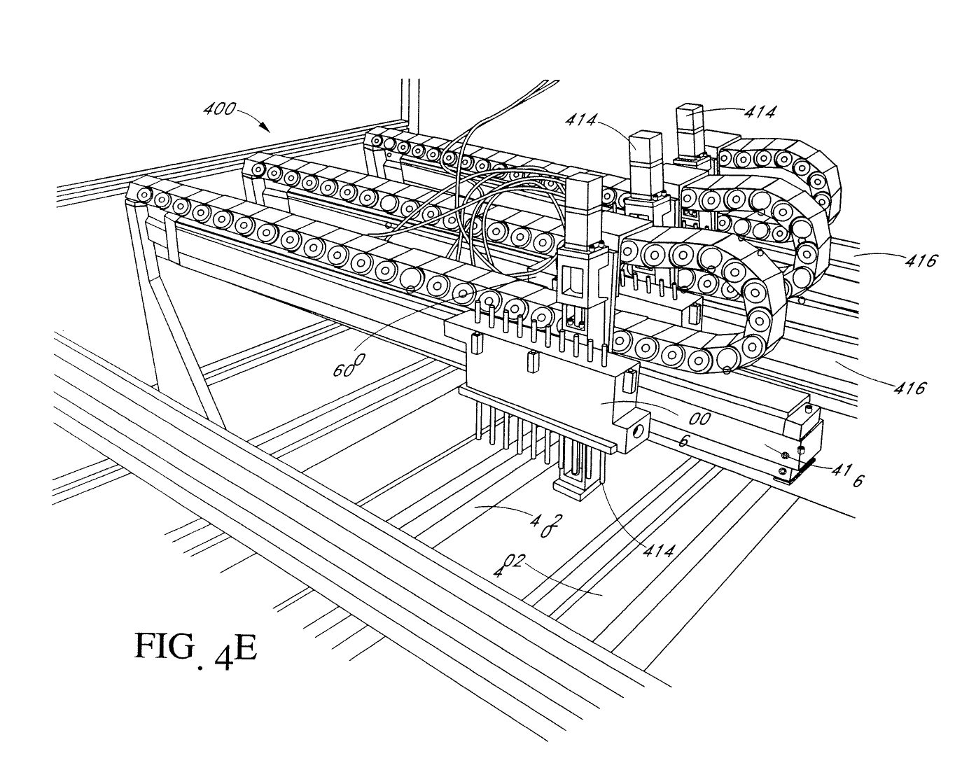

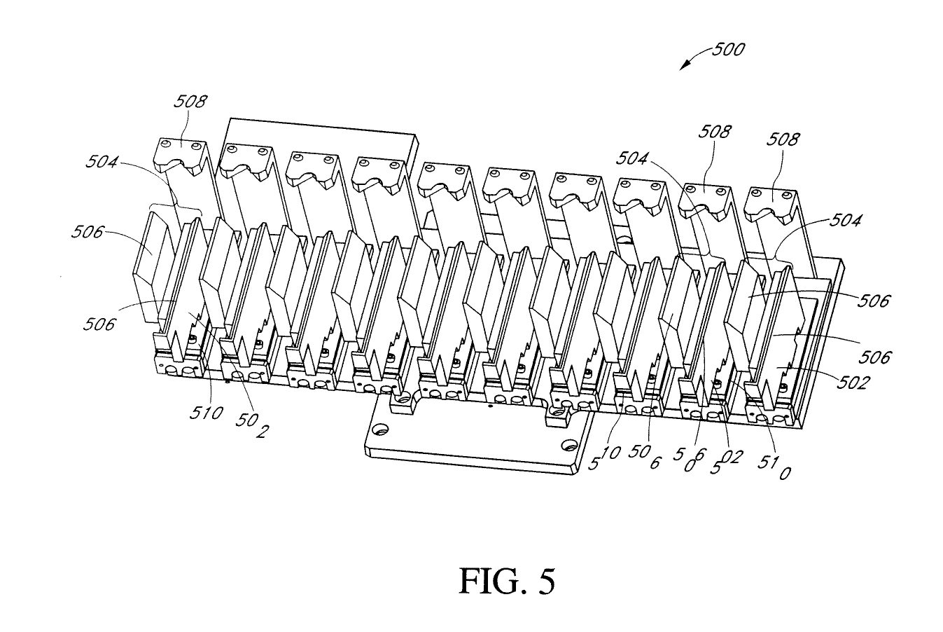

FIGS. 3A-C schematically depict cell culture dissociator 300 according to one embodiment of the invention. In some embodiments, cell culture dissociator 300 is included as a component of a cell culture passaging station (e.g., cell culture passaging station 400, which is schematically depicted in FIGS. 4A-E), whereas in other embodiments, it operates as a stand-alone station (e.g., a cell culture agitation station, etc.) or as a component of another system or sub-system. As shown, cell culture dissociator 300 includes container holder 302, which includes container receiving area 304. Container receiving area 304 is structured to receive cell culture container 306 (shown as a cell culture flask). In other embodiments, container receiving areas are structured to receive more than one cell culture container at the same time. Optionally, a cell culture dissociator includes multiple container holders. When cell culture dissociators are configured to accommodate multiple cell culture containers, cell dissociation can be effected in the containers in parallel. Container holders optionally include angled surfaces that guide cell culture containers into the container receiving areas when the containers are placed into the container receiving areas, e.g., manually or by a robotic gripping device. Examples of these angled surfaces are shown in FIG. 5 (angled surfaces 506 of container holders 502).

Cell culture dissociators also include rotational mechanisms operably connected to the container holders of the devices and effect rotation of cell culture containers disposed in container receiving areas, e.g., to agitate, dissociate, wet, etc. cells contained in the containers. For example, cell culture dissociator 300 includes rotational mechanism 308. Rotational mechanisms are generally configured to rotate container holders between about 0° and about 180° (e.g., between about 0° and about 90°). In certain embodiments, for example, rotational mechanisms include two positions or states. In a first state, cell culture containers (e.g., Corning® RoboFlask™ Cell Culture Vessels (Corning, Inc. Life Sciences, Acton, Mass., USA)) are vertically oriented (as shown in, e.g., FIG. 3B), while in a second state, the containers are horizontally oriented (as shown in, e.g., FIG. 3C). The vertical state typically allows for robot and tip access to the containers. In some embodiments the horizontal state is used for wetting the bottom of a cell culture flask, e.g., during a trypsinizing process. In these embodiments, the wetting of the flask is typically done at a low angular velocity. When set at a higher angular velocity, the container holder holding the flask will generally impact other portions of a cell culture dissociator (e.g., comprising one or more stops, which are described further below) with sufficient force to send shear forces parallel to the bottom of the flask. This process is typically utilized to detach adherent cells from the bottom of the flask after trypsin or another dissociative reagent has been added to the container. Optionally, a material handling component is used to triturate (i.e., pipette cells up and down) clumps of cells to dissociate them from one another.

In some embodiments, rotational mechanisms include counterweights that counter or radially balance out the weight of container holders and cell culture containers disposed in those mechanisms when the rotational mechanisms rotate the container holders. Counterweights allow container holders to rotate under substantially constant force for easy control of angular velocity. In the absence of counterweights, it generally takes more force to start a rotation from a vertical position (shown in FIG. 3A) than to finish the rotation near a horizontal position (shown in FIG. 3C). This typically makes it more difficult to control the angular velocity of container holders when they are rotated into contact with or impact the stops of the cell culture dissociators (described further below). To illustrate, cell culture dissociator 300 includes counterweight 310 operably connected to rotational mechanism 308. In embodiments that include multiple container holders, the holders are optionally disposed symmetrically relative to rotational axes to balance each other out such that separate counterweights are not needed. Controllers are generally operably connected to rotational mechanisms and include logic devices having logic instructions that direct the rotational mechanisms to rotate the container holders at pre-set rates or rates selected by the user.

As referred to above, cell culture dissociators generally include one or more stops. In these embodiments, rotational mechanisms are typically configured to rotate container holders into contact with the stops to effect, e.g., the dissociation of cells from one another and/or from surfaces of the rotated cell culture containers, the agitation of cells in the rotated cell culture containers, etc. For example, cell culture dissociator 300 includes stops 312 that rotational mechanism 308 rotates container holder 302 into contact with. Typically, stops are fabricated from materials that resiliently absorb the impact of the rotated container holders, such as an elastomer or the like.

The cell culture dissociators of the invention also typically include container retention components that are movable relative to the container holders. As an example, cell culture dissociator 300 includes container retention component 314 (shown as a container retention plate) coupled to container holder 302 via slidable coupling 316 (shown as a pneumatic slide). These slidable coupling along with switches generally effect movement of container retention components and container holders relative to one another. Container retention components are structured to retain cell culture containers in substantially fixed positions relative to the container retention components when the containers is disposed in the container receiving area and the container holders are in closed positions (e.g., latched positions). Typically, container retention components are structured to permit access to cell culture containers when the containers are disposed in container receiving areas and the container holders are in these closed positions. Cell culture dissociator 300 is shown in a closed position in, e.g., FIG. 3B. As shown, cell culture container 306 is partially disposed under a portion of container retention component 314 and permits, e.g., tip access to the container. In addition, container retention component 314 retains rotational device 300 in a substantially fixed position when tips are withdrawn from cell culture container 306 by holding cell culture container 306 down during the tip withdrawal process. Controllers are generally operably connected to container holders (e.g., via slidable couplings and/or associated switches). These controllers typically include logic devices comprising logic instructions that direct the container holders to close (e.g., latched positions) or open (e.g., unlatched positions). In an open position, a container receiving area of a container holder is able to receive a cell culture container, e.g., from a robotic gripping device or via manual placement. To illustrate, cell culture dissociator 300 is shown in an opened position in, e.g., FIG. 3A, such that container holder 302 receives cell culture container 306 in a vertical orientation (i.e., on an edge of the container).

ii. Container Positioning Devices

In some embodiments, the systems of the invention include container positioning devices. To illustrate, FIG. 5 schematically shows a perspective view of container positioning device 500 according to one embodiment of the invention. As shown, container positioning device 500 includes multiple container holders 502 that include container receiving areas 504. Container receiving areas 504 are structured to receive cell culture containers (e.g., cell culture container 306). Although container positioning device 500 includes ten container holders 502, other numbers of container holders are also optionally utilized (e.g., 1, 5, 15, 20, 25, etc.). As also shown, container holders 502 include angled surfaces 506, which guide cell culture containers into container receiving areas 504 when the containers are placed into container receiving areas 504, e.g., manually or by a robotic gripping device.

In addition, container positioning device 500 also includes container retention components 508 (shown as a container retention plate) that are movable relative to container holders 502. As described above with respect to cell culture dissociators, container retention components 508 are typically structured to retain cell culture containers in substantially fixed positions relative to container retention components 508 when the cell culture containers are disposed in container receiving areas 504 and container holders 502 are in closed positions relative to container retention components 508. Container retention components 508 are coupled to container holders 502 via slidable couplings 510 (shown as a pneumatic slide). Slidable couplings 510 along with switches (not within view) typically effect movement of container retention components 508 and container holders 502 relative to one another. Optionally, line flow regulators are used on pneumatic slides to regulate the speed of the retracting and extending motion of the containers. If the speed is too high, containers typically impact the stops with excessive force that dislodges the containers from desired positioning. Further, the logic devices (e.g., computers, etc.) of system controllers typically include logic instructions that direct container holders 502 to move to closed positions or to open positions.

In certain embodiments, cell culture dissociators and container positioning devices are operably connected to translational mechanisms that effect the translation of these devices along at least one translational axis. For example, in cell culture passaging station 400 includes cell culture dissociator 300 and container positioning device 500 mounted on translational mechanisms 402 and 404, respectively. During operation, translational mechanisms 402 and 404 independently translate cell culture dissociator 300 and container positioning device 500 in decontamination device 406 between first chamber 408 and second chamber 410, which are components of decontamination device 406. A system controller is typically operably connected to translational mechanisms 402 and 404 and includes logic instructions that direct translational mechanisms 402 and 404 to translate cell culture dissociator 300 and container positioning device 500, respectively, to selected positions along the translational axes of translational mechanisms 402 and 404. As shown, material handling component 412 (shown as a dispensing device) is disposed in first chamber 408 of decontamination device 406. Decontamination devices and material handling component are described further below. In cell culture passaging station 400, cell culture dissociator 300 typically functions as a source cell culture flask locator (i.e., a source of cells to be passaged), while container positioning device 500 generally functions as a destination flask locator that positions cell culture flasks to receive cells from a cell culture flask positioned in the source cell culture flask locator for sub-culturing.

iii. Material Handling Components

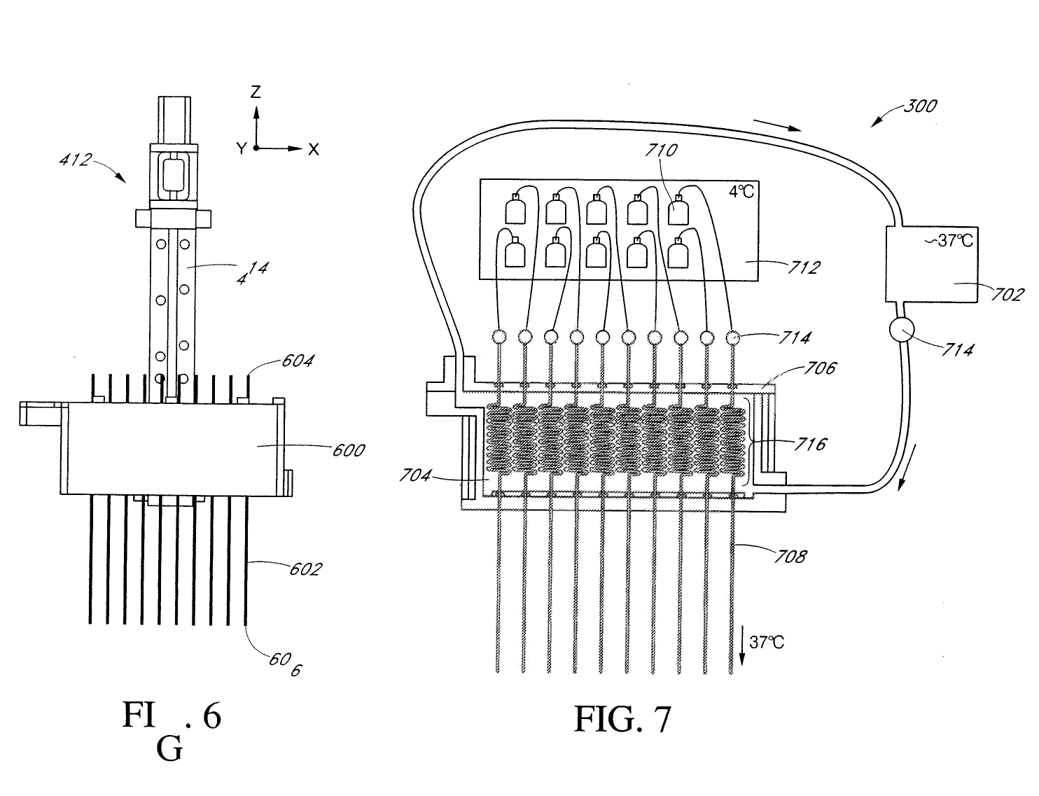

The systems of the invention typically include various types of material handling components. For example, cell culture passaging stations (e.g., cell culture passaging station 400, which is schematically depicted in FIGS. 4A-E) generally include at least one fluidic material transfer component or dispensing device that is configured to transfer fluidic materials (e.g., cell culture media, fluidic reagents, etc.) to and/or from containers positioned in one or more components of the system. Typically, these dispensing devices include at least one conduit having an inlet and an outlet that fluidly communicate with one another. To illustrate, FIG. 6 schematically illustrates a front elevational view of dispense head 600 of dispensing device 412 according to one embodiment of the invention. As shown, dispense head 600 includes multiple conduits 602 that each include inlet 604 and outlet 606 that fluidly communicate with one another. Conduits 602 are structured as tips that can be inserted into cell culture containers (e.g., cell culture container 306) positioned in cell culture dissociator 300 and container positioning device 500 such that fluids can be dispensed into and/or aspirated from those containers through conduits 602. Although dispense head 600 includes ten tips, dispense head having other numbers of tips are also optionally utilized. Conduits 602 are generally inserted through elastomeric septums, gaskets, or other re-sealable (e.g., self-sealing) ports of these containers to establish fluid communication with the containers. Accordingly, conduits 602 can have non-coring profiles to minimize damage to, e.g., flask septums upon insertion into and withdrawal from these containers. In addition, tips are also typically coated with ceramic or another coating that provides for chemical inertness or compatibility with fluidic materials contained in the cell culture containers. The inlets of dispensing device conduits typically fluidly communicate with one or more fluid sources. In addition, fluid conveyance devices (e.g., peristaltic pumps, etc.) are generally operably connected to these conduits and/or to the fluid sources to effect the conveyance of fluidic materials from the fluid sources. Fluid sources and fluid conveyance devices discussed further below, e.g., with reference to FIG. 7.

Dispense heads are generally mounted to one or more translational mechanisms that are capable of translating the dispense heads along one or more translational axes. To illustrate with reference to, e.g., FIGS. 4A, 4B, 4E, and 6, dispense head 600 is coupled to Z-axis translational mechanism 414 and to Y-axis translational mechanism 416. Z-axis translational mechanism 414 is configured to translate dispense head 600 along the Z-axis so that conduits 602 can be inserted into and removed from cell culture containers positioned in cell culture dissociator 300 and container positioning device 500. In contrast, Y-axis translational mechanism 416 is configured to translate dispense head 600 along the Y-axis such that dispense head 600 can be moved between culture rotational device 300 and container positioning device 500. As further shown in FIG. 4E, for example, cell culture passaging station 400 includes multiple dispense heads 600, Z-axis translational mechanisms 414, and Y-axis translational mechanisms 416.

In some embodiments, dispense heads include chambers through which at least segments of the conduits are disposed. In these embodiments, dispensing devices also typically include thermal regulation components that fluidly communicate with the chambers (e.g., heat exchange chambers) to regulate the temperature of fluids that are conveyed through the conduits. To illustrate, FIG. 7 schematically depicts dispensing system 700, which include thermal regulation component 702. As shown, thermal regulation component 702 includes a fluid recirculation bath that fluidly communicates with chamber 704 of dispense head 706 (shown in a cross-sectional view). As also shown, conduits 708 fluidly communicate with fluid sources 710. Pumps 714 are configured to effect the conveyance of fluidic materials from fluid sources 710 through conduits or tips 708, e.g., via tubing that connects fluid sources 710 and conduits 708 of dispense head 706. Fluid source storage device 712 (e.g., a refrigeration device, etc.) stores fluid sources 710 at a selected temperature (e.g., about 4° C. in certain applications), e.g., to minimize the degradations of fluidic reagents contained in fluid sources 710 prior to being dispensed. During operation, pump 714 of thermal regulation component 702 effects the recirculation of another fluid (e.g., an antifreeze solution, etc.) that is substantially maintained at another selected temperature (e.g., about 37° C. for certain cell culturing applications). This recirculated fluid functions as a heat transfer medium. In particular, as fluidic reagents are conveyed from fluid sources 710 through segments 716 of conduits 708, those fluidic reagents attain a temperature that is closer to that of the fluid recirculated through chamber 704 than to the temperature of the fluid in fluid storage device 712. Preferably, the fluidic reagents that flow through segments 716 substantially attain the temperature of the fluid recirculated through chamber 704 of dispense head 706 by thermal regulation component 702.

To further illustrate, a fluid recirculation bath of a thermal regulation component maintains an antifreeze solution at a temperature that is slightly above 37° C. in certain embodiments. The antifreeze solution is continuously pumped through a dispense head chamber at a high flow rate to insure that a uniform temperature is maintained inside the chamber (i.e., approximately 37° C.). In contrast, fluid sources containing fluidic reagents used in cell culture passaging applications are maintained at about 4° C. in a refrigeration device. One or more peristaltic pumps pump selected amounts of these fluidic reagents to cell culture flasks disposed in culture rotational devices and/or in container positioning devices. As these reagents are flowed through the chamber of the dispense head in the tips, their temperature is raised from 4° C. to 37° C. just as they are dispensed from the tips. Certain cells are stored or grown in media at a temperature of about 37° C. If the temperature of these cells deviates too far from 37° C., they may go into shock, which adversely affects their growth rate. In contrast, fluidic reagents (e.g., media components, etc.) used for cell or tissue culture are stored at 4° C., in some embodiments, to minimize the degradation of these reagents over time. Accordingly, these reagents are heated from 4° C. to 37° C. prior to contacting the cells in these embodiments. This mechanism heats these cell or tissue culture reagents on a continuous “as required” basis, which maximizes the amount of reagents stored at the colder temperature to minimize the amount of reagent that would otherwise be degraded at elevated temperatures.

Dispense heads can be fabricated from various materials, including various ceramic, metallic, and/or polymeric materials. In certain embodiments, for example, dispense heads are fabricated from aluminum such that the heat exchange chambers are sealed within body structures of the heads. Component fabrication is described further below.

In certain embodiments, segments of conduits disposed in the chambers of dispense heads include coiled structures. Segments 716 of conduits 708 shown in FIG. 7 schematically illustrate one of these embodiments. Coiled structures are typically used to maximize the length of the conduits disposed within the chambers of the dispense heads so that the ratio of conduit or tip surface area to recirculation fluid volume is maximized. To adequately compensate for heat loss, a length of the conduit included in a coiled structure is typically at least about 167 mm in some embodiments. However, to further compensate for such loss, the coiled length of conduit used in the dispense head chamber is typically at least about 350 mm, and more typically at least about 375 mm (e.g., at least about 390 mm, at least about 400 mm, at least about 410 mm, etc.). The length 167 mm is derived from the OD and wall thickness of the conduit or tip. An example illustrating how this length was calculated is provided below. If a larger conduit or tip with thicker walls is utilized, for example, the length would typically have to be increased.

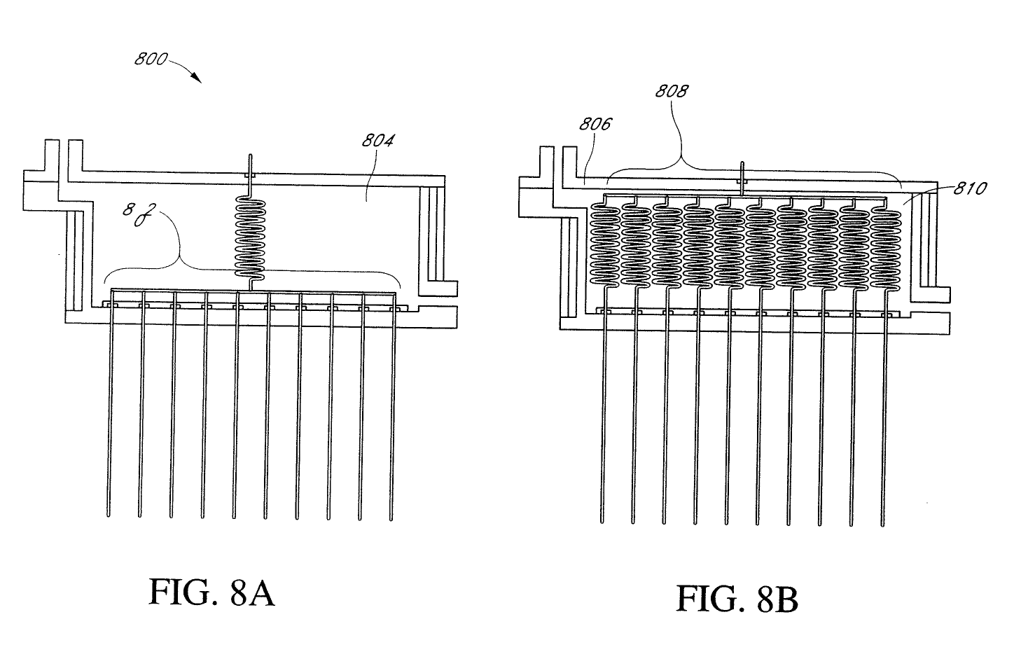

In some embodiments, dispense heads include one or more manifolds that fluidly communicate with conduits or tips. To illustrate, FIG. 8A schematically shows a cross-section through dispense head 800 that includes manifold 802 disposed within chamber 804 of dispense head 800. To further illustrate, FIG. 8B schematically illustrates another exemplary embodiment of a dispense head that includes a manifold. In particular, dispense head 806 (also shown in a cross-sectional view) includes manifold 808 disposed within chamber 810 of dispense head 806.

Additional material handling components and related methods that are optionally utilized in the systems of the invention are also described further herein. For example, devices and systems for dispensing and/or removing materials from multi-well containers in addition to methods of dispensing substantially uniform concentrations of cell culture media are described below.

iv. Decontamination Devices

The invention also provides devices that can be used to minimize contamination by isolating those components of the system that are most vulnerable to contaminants. Other pre-existing approaches to minimizing contamination have included enclosing the entire system in a Class II-type cabinet and to use disposable reagent or sample containers (e.g., cell culture flasks, etc.). For many applications, however, this approach is impractical, because users are required to wear new clean suits each time access to the system components is needed, such as for service or maintenance, or the like, which can be between about one to five times a day in certain cases. In addition, because these systems are typically composed of many parts, it is difficult to ensure that all of these parts are suitably clean before they are placed in the clean room environment. Accordingly, the issue of eliminating contamination is addressed in the systems of the invention by, e.g., enclosing only selected portions (e.g., dispensing devices, etc.) of the systems in a substantially sterile or clean room environments in certain embodiments. In this approach, only those parts that are moving in and out of those environments generally need to be cleaned. To illustrate, one such part may be a cell culture container or flask. Using pre-existing approaches, users who wish to enter the clean room housing the entire system must typically wear a clean room suit and first enter an ante-chamber, where they are exposed to high velocity streams of clean air to filter our or otherwise remove contaminants that they may be carrying. This same approach is utilized in certain embodiments of the systems described herein, but only for components of the operating system (e.g., cell culture flasks, multi-well containers, reagent containers, disposable tips, etc.). This reduces the frequency that users need to access clean room environments (e.g., in clean room suits) within the systems relative to approaches that involve the enclosure of the entire system.

More specifically, in some embodiments, the systems of the invention include one or more decontamination devices that each includes a first chamber that at least transiently houses at least one system component (e.g., a cell culture dissociator, a material handling component, a container positioning device, etc.). The first chamber is generally comprises a HEPA or other filtration system that maintains a substantially sterile environment (e.g., a Class II-type environment) in the first chamber. These decontamination devices also each include a second chamber (e.g., an ante-chamber, etc.) that communicates with the first chamber such that one or more containers (e.g., cell culture containers, multi-well containers, etc.) are capable of being translocated between the first and second chambers, e.g., in an automated manner using a robot gripping mechanism, a translocation mechanism, etc. These decontamination devices also each include at least one decontamination component that communicates at least with the second chamber. These decontamination components are configured to substantially decontaminate one or more surfaces of the containers when the containers are disposed in the second chamber (i.e., before the containers are translocated from the second chambers into the first chambers). In certain embodiments, for example, decontamination components include gas sources that are configured to flow gas (e.g., air, an inert gas, etc.) into the second chambers with sufficient velocities to substantially remove contaminants from the surfaces of the containers when the containers are disposed in the second chambers. Other decontamination components are also optionally adapted for use in the systems described herein, such as radiation sources that are configured to irradiate container surfaces to effect decontamination. To further illustrate, other exemplary decontamination components that are optionally utilized include, e.g., decontamination fluid misters, UV lamps, thermal decontamination devices, plasma cleaning devices, or the like.

Referring now to FIGS. 4A, 4B, and 4D, decontamination device 406 includes first chamber 408, which encloses dispensing device 412 in a substantially sterile environment. As also shown, decontamination device 406 includes second chamber 410 (schematically shown as an ante-chamber) and decontamination component 411 (schematically shown as a high velocity clean air blower) that blows or blasts high velocity clean air into second chamber 410 to remove contamination, e.g., from the septum and outer walls of cell culture flasks. In this manner, any bacteria or other contamination that may remain adhered to these flasks after going through this procedure will typically remain on the flasks and will not contaminate dispensing device 412, because the velocity of the air flow in first chamber 408 is much lower than that in second chamber 410. In some embodiments, decontamination devices also include decontamination fluid misters that mist cell culture flasks with a decontamination fluid (e.g., 70% ethanol, etc.) to effect further decontamination of the flasks in the ante-chambers following the initial air blasts. In these embodiments, the flasks are then typically blasted with high velocity clean air to evaporate or otherwise remove the decontamination fluid from the surfaces of the flasks before the flasks are processed further. During operation, translational mechanisms 402 and 404 independently translate cell culture dissociator 300 and container positioning device 500 in decontamination device 406 between first chamber 408 and second chamber 410.

In some embodiments, ante-chambers communicate with the HEPA-enclosed chambers via passageways. These passageways optionally include movable sealing mechanisms (e.g., an air lock, etc.) that are structured to reversibly separate these first and second chambers from one another.

C. Robotics

The systems of the invention typically include one or more robotic components that, at least in part, effect system automation. To illustrate, although other numbers are optionally utilized, a system of the invention generally includes from about one to about 10 robotic devices. Typically, these robots are configured for rotation about an axis and each have a rotational range of about 360 degrees. In addition, each robot typically adjusts vertically and horizontally to align with relatively higher or lower work positions. Moreover, each rotational robot generally has a robotic arm that extends and/or retracts from the robot's rotational axis. Accordingly, each rotational robot has an associated rotational reach, e.g., defining how far out from the rotational axis the robot is capable of operating. As described above, this rotational reach defines a work perimeter, e.g., a circular work perimeter, for that robot.

In addition, each robotic arm typically has a robotic gripper mechanism. For example, a gripper mechanism is used to grasp objects for transport between selected positions with a system. In certain embodiments, for example, gripper mechanisms are configured to removably grasp multi-well containers, such as standard 96, 384, or 1,536 well plates. Gripper mechanisms are also optionally configured to grasp other types of objects, including without limitation, custom sample holders, reaction vessels, reaction blocks, cell culture containers or flasks, crucibles, petri dishes, test tubes, test tube arrays, vial arrays, among many others. Robotic arms and gripper mechanisms are typically operated pneumatically, hydraulically, magnetically, or by other means known in the art. Optionally, gripper mechanisms are coupled to robotic arms via a breakaway or other deflectable member that is structured to deflect when the gripper mechanism contacts an object with a force greater than a preset force, e.g., to minimize the risk of damage to the rotational robot and the object. Exemplary robotic gripping devices that are optionally adapted for use in the systems of the invention are described further in, e.g., U.S. Pat. No. 6,592,324, entitled “GRIPPER MECHANISM,” issued Jul. 15, 2003 to Downs et al. and International Publication No. WO 02/068157, entitled “GRIPPING MECHANISMS, APPARATUS, AND METHODS,” filed Feb. 26, 2002 by Downs et al., which are both incorporated by reference.

In some embodiments, the robotic gripping devices include sensors (e.g., optical sensors, etc.), e.g., for detecting containers or other objects being transported and the direction a particular sample container should be inserted into a device, such as a plate reader. In addition, a sensor optionally determines a location of gripper mechanisms relative to objects to be transported.

Suitable robots are available from various commercial suppliers known in the art. In some embodiments, for example, Stäubli RX-60 robots (provided by Stäubli Corporation of South Carolina, U.S.A.) are utilized in the systems of the invention. Such robots are highly accurate and precise, e.g., typically to within about one one-thousandth of an inch. Other robot models from this or other suppliers are also optionally used. A variety of other robotic instrumentation that is optionally adapted for use with the present invention is available from, e.g., the Zymark Corporation (Zymark Center, Hopkinton, Mass.), which utilize various Zymate systems, which can include, e.g., robotics and fluid handling modules. Similarly, the common ORCA® robot, which is used in a variety of laboratory systems, e.g., for microtiter tray manipulation, is also commercially available, e.g., from Beckman Coulter, Inc. (Fullerton, Calif.).

The robots and associated work perimeters and station locations are typically attached to one or more frames that support the system components. To illustrate, weldments, aluminum extrusions, etc. are optionally used to provide support frames with optics table tops or other support surfaces for mounting various devices, e.g., cell culture passaging stations, incubators, detectors, and the like. Table tops such are these are commercially available from various suppliers, including Melles Griot, Inc. (Carlsbad, Calif., USA).

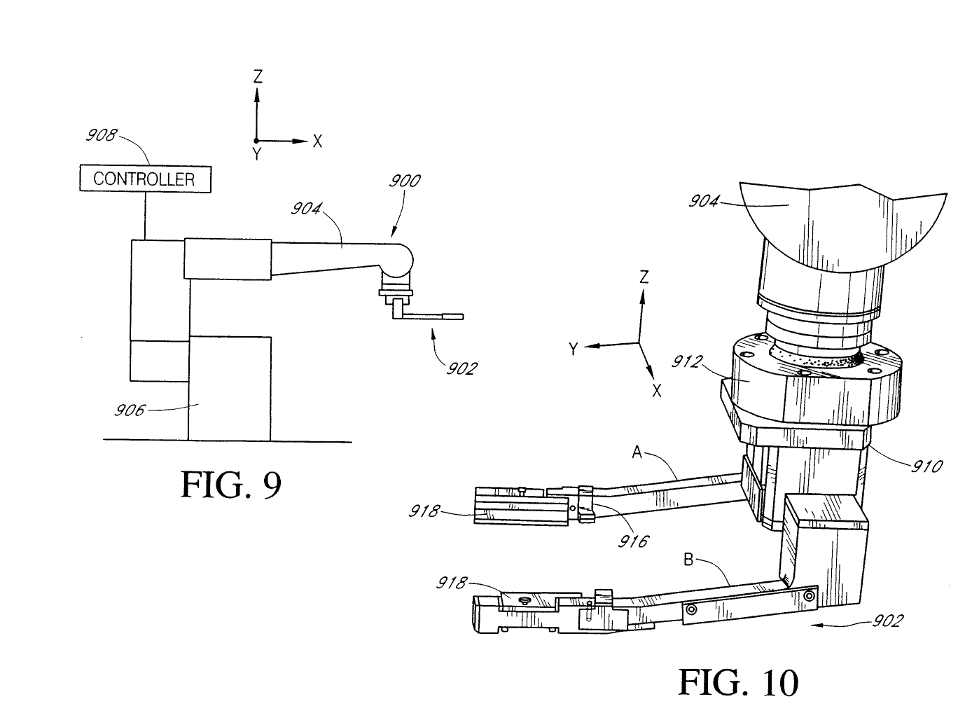

To further illustrate, FIG. 9 schematically depicts robotic gripping device 900 from a side elevational view according to one embodiment. Robotic gripping device 900 is an automated robotic device, e.g., for accurately and securely grasping, moving, manipulating and/or positioning objects. The design of robotic gripping device 900 is optionally varied to accommodate different types of objects. For example, robotic gripping device 900 is optionally manufactured to grasp sample containers or plates (e.g., cell culture flasks, microwell plates, or the like). Other exemplary objects include, e.g., fermentation sample vessels, fermentation apparatus, centrifuge rotors, etc.

In the embodiment illustrated in FIG. 9, robotic gripping device 900 includes gripper mechanism 902 movably connected to boom 904, which is movable relative to base 906. Controller 908, which optionally includes a general purpose computing device, controls the movements of, e.g., gripper mechanism 902 and boom 904 in a work perimeter that includes one or more stations that can receive and support selected objects.

Boom 904 is configured to extend and retract from base 906. As described above, this defines the work perimeter for robotic gripping device 900. Stations (e.g., the cell culture passaging stations described above) are positioned within the work perimeter of boom 904 as are hand-off areas or other areas that are configured to support or receive objects grasped and moved by gripper mechanism 902. For example, sample containers are positioned on a station shelf or container positioning device and can be grasped by gripper mechanism 902 and moved to another position by boom 904.

Referring now to FIG. 10, one embodiment of gripper mechanism 902 is illustrated. Grasping arm A and grasping arm B extend from gripper mechanism body 910. Although the embodiments described herein include two arms for purposes of clarity of illustration, the gripper mechanisms of the invention optionally include more than two arms, e.g., about three, about four, about five, about six, or more arms. Further, although in certain embodiments, gripper mechanism arms are structured to grasp objects between the arms, other configurations are also optionally included, e.g., such that certain objects can be at least partially, if not entirely, grasped internally, e.g., via one or more cavities disposed in one or more surfaces of the particular objects.

As further shown in FIG. 10, grasping mechanism body 910 is connected to a deflectable member, such as breakaway 912, which is deflectably coupled to boom 904. Breakaway 912 is typically structured to detect angular, rotational, and compressive forces encountered by gripper mechanism 902. The breakaway acts as a collision protection device that greatly reduces the possibility of damage to components within the work perimeter by, e.g., the accidental impact of gripper mechanism 902 or grasping arms A and B with objects. For example, when gripper mechanism 902 impacts an object, breakaway 912 will deflect, thereby also causing gripper mechanism 902 to deflect. To further illustrate, deflectable members of robotic gripping devices generally deflect when the gripper mechanism contacts an object or other item with a force greater than a preset force. The preset force typically includes a torque force and/or a moment force that, e.g., ranges between about 1.0 Newton-meter and about 10.0 Newton-meters. When controller 908 detects the deflection, it generally stops movement of the robotic gripper mechanism. In one embodiment, breakaway 912 is a “QuickSTOP™” collision sensor manufactured by Applied Robotics of Glenville, N.Y., U.S.A. Breakaway 912 is typically a dynamically variable collision sensor that operates, e.g., on an air pressure system. Other types of impact detecting devices are optionally employed, which operate hydraulically, magnetically, or by other means known in the art. In certain embodiments, breakaways are not included in robotic gripping devices used in the systems of the invention. In these embodiments, gripper mechanisms are typically directly coupled to robotic booms.

As also shown, body 910 connects grasping arms A and B to breakaway 910. When directed by controller 908, body 910 moves grasping arms A and B away from or toward each other, e.g., to grasp and release objects. In one embodiment, body 910 is manufactured by Robohand of Monroe, Conn., U.S.A. Typically, the grasping arms are pneumatically driven, but other means for operating the arms are also optionally utilized, such as magnetic- and hydraulic-based systems.

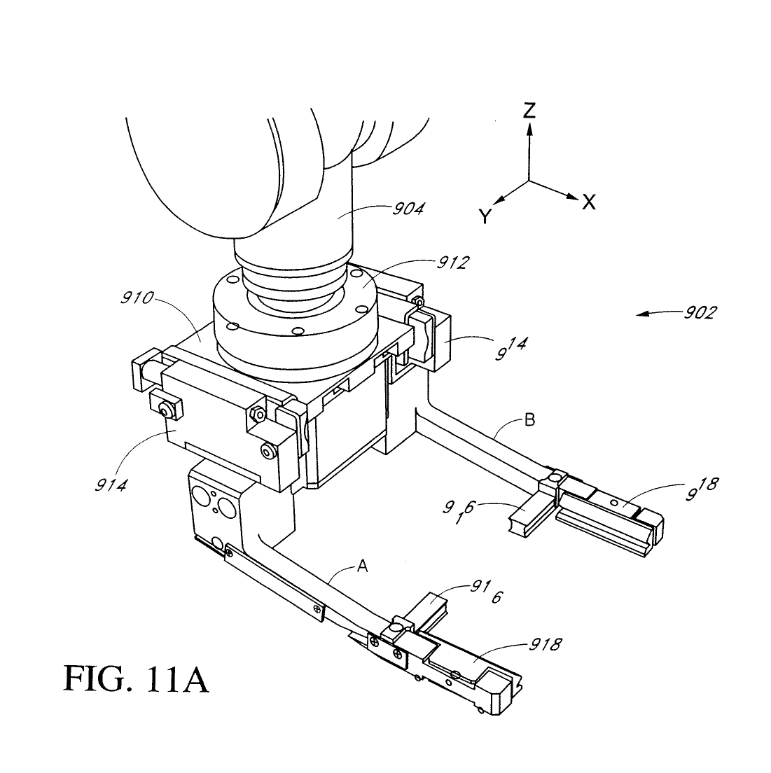

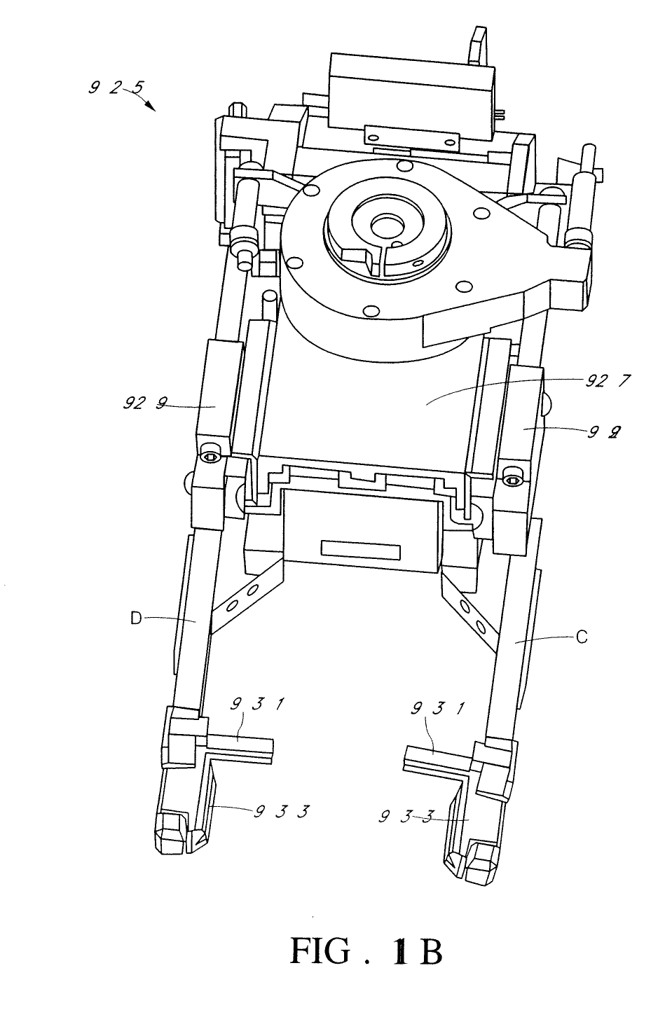

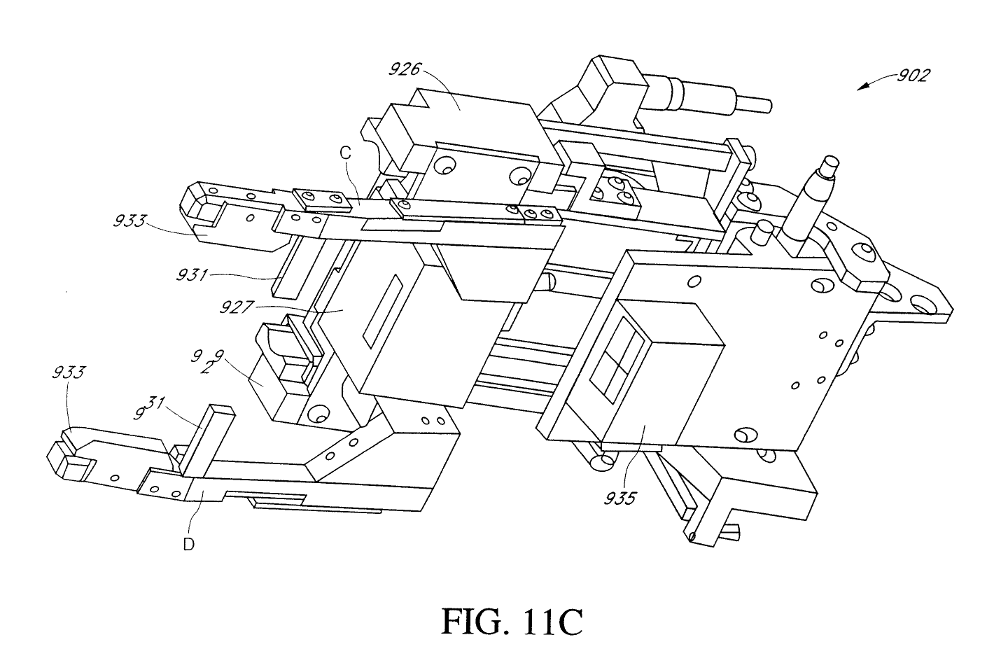

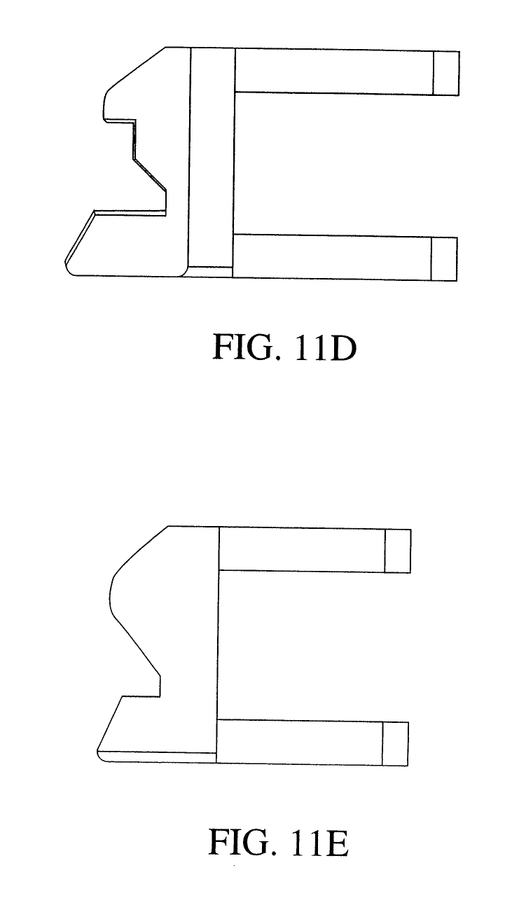

In other embodiments, grasping arms are resiliently coupled to robotic booms such that when an object contacts stops on the grasping arms, the arms reversibly recede from an initial position, e.g., to determine a y-axis position of an object prior to determining the x-axis and z-axis positions of the object. One of these embodiments is schematically illustrated in FIG. 11A. In particular, FIG. 11A schematically depicts one embodiment of gripper mechanism 902 that includes arms A and B resiliently coupled to body 910 via slidable interfaces 914. Slidable interfaces typically include springs, which resiliently couple, e.g., grasping arms to grasping mechanism bodies. Such resiliency is optionally provided by other interfaces that include, e.g., pneumatic mechanisms, hydraulic mechanisms, or the like. As further shown, arms A and B include stops 916 and pivot members 918. As mentioned, the embodiment of gripper mechanism 902 schematically illustrated in FIG. 11A is optionally used to determine the y-axis position of an object prior to grasping the object between the arms, that is, prior to determining the x-axis and z-axis positions of the object. As further shown in FIG. 11A, gripper mechanism 902 is connected to boom 904 via breakaway 912. Breakaways are described in greater detail above.

To further illustrate, FIGS. 11 B and C schematically show grasping mechanism 925 from top and bottom perspective views, respectively, according one embodiment. As shown, grasping mechanism 925 includes arms C and D resiliently coupled to body 927 via slidable interfaces 929 similar to gripper mechanism 902 described above. As also shown, arms C and D include stops 931 and pivot members 933. FIG. 11D schematically shows pivot member 933 from a front elevational view. Pivot member 933 is fabricated to accommodate or compensate for various container skirt or rib heights or thicknesses (e.g., about 1 mm, about 1.5 mm, about 2.0 mm, about 2.5 mm, about 3 mm, about 3.5 mm, and/or greater thicknesses) including the skirt heights of certain cell culture containers (e.g., Corning® RoboFlask™ Cell Culture Vessels (Corning, Inc. Life Sciences, Acton, Mass., USA), etc.). For example, certain cell culture containers include ribs that are designed to help them stand upright without external support. Pivot member 933 can typically accommodate these types of ribs. FIG. 11E schematically illustrates pivot member 918 from gripper mechanism 902 from a front elevational view. Grasping mechanism 925 also includes in-line bar code reader 935, mounted on a height and angled adjustable mechanism of grasping mechanism 925. Bar code reader 935 is configured to read bar codes disposed on containers when bar code reader 935 is within sufficient proximity to the container, such as when the containers are grasped by arms C and D of grasping mechanism 925. Bar codes are typically used to track the location of containers in the systems of the invention. Other tracking methods known to persons of skill in the art are also optionally utilized. Although not shown, grasping mechanism 925 is typically coupled to a boom of a robotic gripping device in the systems described herein.

The robots of the systems described herein are typically used to transport one or more sample containers between locations in the systems. In some embodiments, for example, robots transfer samples disposed in sample containers from one work perimeter to another work perimeter, e.g., via a transfer station. To transfer between adjacent work perimeters, a first robot generally retrieves a sample container, positions the container at a transfer station, and then a second robot from an adjacent work perimeter retrieves the container from the transfer station. Alternatively, robots are configured to directly transfer a sample plate from one robot to another.

In addition, the robots generally transfer sample containers and other objects between station locations within the associated work perimeter of the robot. In this manner, the sample containers are transported to various sub-systems or devices of the systems, e.g., for further processing, measurement, detection, etc.

Although the systems of the invention are primarily automated, certain functionalities are optionally performed manually. For example, an operator optionally manually introduces a particular sample container into a system, e.g., by placing the container onto a table device, holding area, or the like. To illustrate, holding areas 232 and 234 (shown in FIG. 2) are optionally used to manually introduce sample containers into system 200, that is, into work perimeter 206 and 214 respectively. Rotational robots optionally retrieve the sample containers from the manual holding areas. It is then optionally moved to a container storage device or other station location, or moved to a transfer station, such as transfer station 242 or 244, e.g., to be retrieved by another robot. The rotational robot that retrieves the sample container from the holding area or transfer station typically moves the container into any of its associated station locations, e.g., for further processing by the device associated with that station. For example, a rotational robot optionally positions a sample container within sensory communication of the detectors included in a system or deposits the container relative to a dispensing device. To facilitate such manual operation, the operator typically uses a basic command set to introduce, move, and process individual sample holders. Any combination of manual and automated processes is contemplated within the present invention. However, sample containers are also optionally introduced into systems automatically, e.g., from a storage device disposed outside of the work perimeters of the systems using a conveyor or other mechanism. In this case, a central controller or a controller coupled to the storage device is typically used to direct which sample containers are introduced into the systems.

Certain robotic gripping devices used in the systems of the invention can be used to effect the agitation of cell culture containers. In certain embodiments, for example, this agitation is a gentle “cross motion” (e.g., forward-back translation in one axis and forward-back translation in second axis normal to vertical axis). In some embodiments, a rotation motion about the vertical axis with sinusoidal pulses that set a wave pattern in the containers can be utilized. Typically, the goal is to shake the cells for uniform distribution in the container without wetting the top of the container. For example, if the top of the container is wet, then a non-intrusive cell counter or microscope can have difficulty resolving the cells in the container.

In addition to rotational robots, other automated robotic devices are also typically used in the systems of the invention. As also described above, for example, systems include translational mechanisms operably connected to cell culture dissociators in certain embodiments. These translational mechanisms are typically configured to move the cell culture dissociator along a translational axis. In these embodiments, controllers are generally operably connected to the translational mechanisms and comprise logic instructions that direct the translational mechanisms to translate the cell culture dissociators to selected positions along the translational axis.

D. Assaying Components

The present invention provides sample assaying components that can support a broad range of assay formats, including screens for compounds with desired properties. The systems of the invention are typically highly automated with minimal user intervention for repeated usage at high throughput in, e.g., laboratory and industrial settings. The systems described herein are also highly adaptable such that a variety of samples and sample assays can be accommodated by the systems to acquire information about the samples. For example, certain other automated tissue culturing or compound profiling systems are designed to automate the process of seeding the cells, incubation, trypsination, cell counting and viability determination, splitting of cell lines, and collection and plating of cells. In certain embodiments, the automated compound profiling systems of the invention are able to perform all these tasks, but unlike many of these pre-existing systems, the systems of the invention also have the capability to test the cell-lines against compounds, e.g., by including assaying components in the system. In some of these embodiments, for example, the assaying components include non-pressure-based fluid transfer probes, such as pin tools. The purpose of using such non-pressure-based fluid transfer probes is to transfer test compounds or other test reagents from test reagent plates into assay plate containing cells (e.g., assay plates that include 96-wells, 384-wells, 1536-wells, or even higher well densities). To further illustrate, if twenty-one hundred compounds have previously been proven to be toxic to certain types of tumors, eight different dilutions of the twenty-one hundred compounds (16,800 compounds total) may exposed to, e.g., two, twenty-five, fifty, or sixty to one hundred cell lines using these non-pressure-based fluid transfer probes. Once a cell line has been exposed to a compound it is possible to determine such factors as whether the compound is toxic to the cell line, whether the compound is activating a specific signal transduction pathway, etc. Assaying components that are optionally adapted for use in the systems of the present invention are also described in, e.g., U.S. patent application Ser. No. 10/911,388, entitled “NON-PRESSURE BASED FLUID TRANSFER IN ASSAY DETECTION SYSTEMS AND RELATED METHODS,” filed Aug. 3, 2004 by Evans et al., which is incorporated by reference.

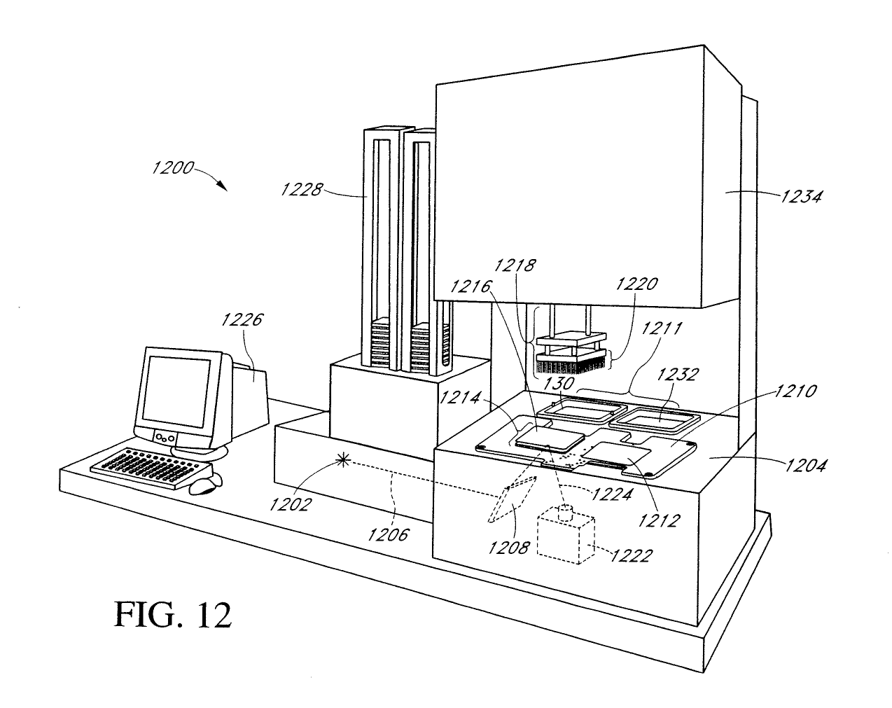

To further illustrate, FIG. 12 schematically shows an assaying component from a perspective view according to one embodiment of the invention. As shown, assaying component 1200 includes electromagnetic radiation source 1202, which is schematically depicted as a laser. Other electromagnetic radiation sources are also optionally adapted for use in the systems of the invention, including electroluminescence devices, laser diodes, light-emitting diodes (LEDs), incandescent lamps, arc lamps, flash lamps, fluorescent lamps, and the like. Assaying component 1200 also includes sample assaying region 1204, which is configured to receive source electromagnetic radiation 1206 from electromagnetic radiation source 1202 via mirror 1208. Various optical systems are optionally utilized or adapted for use in the systems of the invention. Exemplary optical systems are described or referred to herein. Other suitable optical systems are known in the art and will be apparent to those of skill in the art.

In some embodiments, sample assaying region 1204 includes container positioning device 1210, which includes container stations 1212 and 1214 that are each structured to position container 1216 (shown as a multi-well container) relative to fluid transfer device 1218. Fluid transfer device 1218 includes non-pressure-based fluid transfer probe 1220 (shown as a pin tool). Sample assaying region 1204 also includes transfer probe washing station 1211, which includes wash reservoirs 1230 and 1232 for washing non-pressure-based fluid transfer probe 1220. Fluid transfer device 1218 is configured to transfer fluid in at least one selected region (e.g., sample assaying region 1204, as shown) of assaying component 1200. In certain embodiments, non-pressure-based fluid transfer probe 1220 is removably attached to a chassis of fluid transfer device 1218. As also shown, assaying component 1200 also includes detector 1222 configured to detect sample electromagnetic radiation 1224 received from sample assaying region 1204. Various detectors are optionally adapted for use in the assaying components of the invention including, e.g., charge-coupled devices (CCDs), intensified CCDs, photomultiplier tubes (PMTs), photodiodes, avalanche photodiodes, etc. Hood 1234 of assaying component 1200 moves to enclose sample assaying region 1204 to exclude, e.g., electromagnetic radiation other than source and sample electromagnetic radiation 1206 and 1224, respectively, or other contaminates that may bias assay results from sample assaying region 1204. In certain embodiments, fluid transfer devices and detectors are included in separate stations of the systems of the invention.

Assaying component 1200 also includes controller 1226 (shown as computer) that is typically operably connected to, e.g., electromagnetic radiation source 1202, fluid transfer device 1218, and detector 1222. Optionally, controller 1226 is also operably connected to other system components. The controllers of the invention typically include at least one logic device (e.g., a computer such as the one illustrated in FIG. 12) having one or more logic instructions that direct operation of one or more components of the system. Also shown is container storage component 1228, which stores containers before and/or after being assayed. All of these system components are described in greater detail below.

i. Non-Pressure-Based Fluid Transfer Probes and Fluid Transfer Devices

One of the advantages of the assaying components of the present invention is the reproducible transfer of fluids at higher levels of throughput than can be achieved with more conventional systems such as those that rely solely upon pressure-based methods of fluid transfer. For example, pipette tips commonly used in various pipetting devices often become completely or partially obstructed which can yield inaccurate delivery of selected fluid volumes, if at all, which ultimately may bias assay results. In addition, assays or screens performed utilizing these types of pressure-based devices often necessitate replacing pipette tips at various steps in the particular protocol, which further limits the throughput of the assay being performed. Furthermore, the cost of disposable pipette tips can significantly add to the overall cost of running a large number of assays. Although pressure-based fluid transfer devices are also optionally used in the systems described herein, the assaying components described herein can avoid the shortcomings of these devices by utilizing non-pressure-based fluid transfer probes to effect reliable fluid transfer.

As referred to above, the non-pressure-based fluid transfer probes used in the assaying components of the invention are optionally pin tools. The pins tools utilized in these system components generally include a support structure having at least one attachment feature that can removably attach the pin tool to a chassis or other structural component of the fluid transfer device of the assaying component. Attachment features can be in the form of hooks or hook mounts that hook onto corresponding components of the chassis. Any other functionally equivalent attachment feature can also optionally be utilized or adapted for use in the assaying components described herein. In addition, the pin tools of the assaying component of the invention also include pin tool heads that have at least one pin attached to the head. Pins are typically free floating in pin tool heads or resiliently coupled to pin tool heads by a resilient coupling, such as a spring, an elastomer, or other such coupling device or material known in the art, to minimize the risk of damaging a component of the system and/or a sample container or support if a pin contacts the container or support. Pin tool heads are typically removably attached to the support structures of pin tools. This facilitates exchanging, e.g., pin tool heads having different pin densities and/or configurations, etc. Pin tool heads are generally removably attached to the support structure by one or more attachment components, such as set screws, spring ball sockets, and/or the like. In some embodiments, pin tool heads further include a rotational adjustment feature (e.g., a screw or the like) such that pin tool heads are capable of rotating relative to corresponding support structures, e.g., to align the pin tool heads with various containers or supports and/or various system components. Rotational adjustment features or mounts are described in greater detail below.

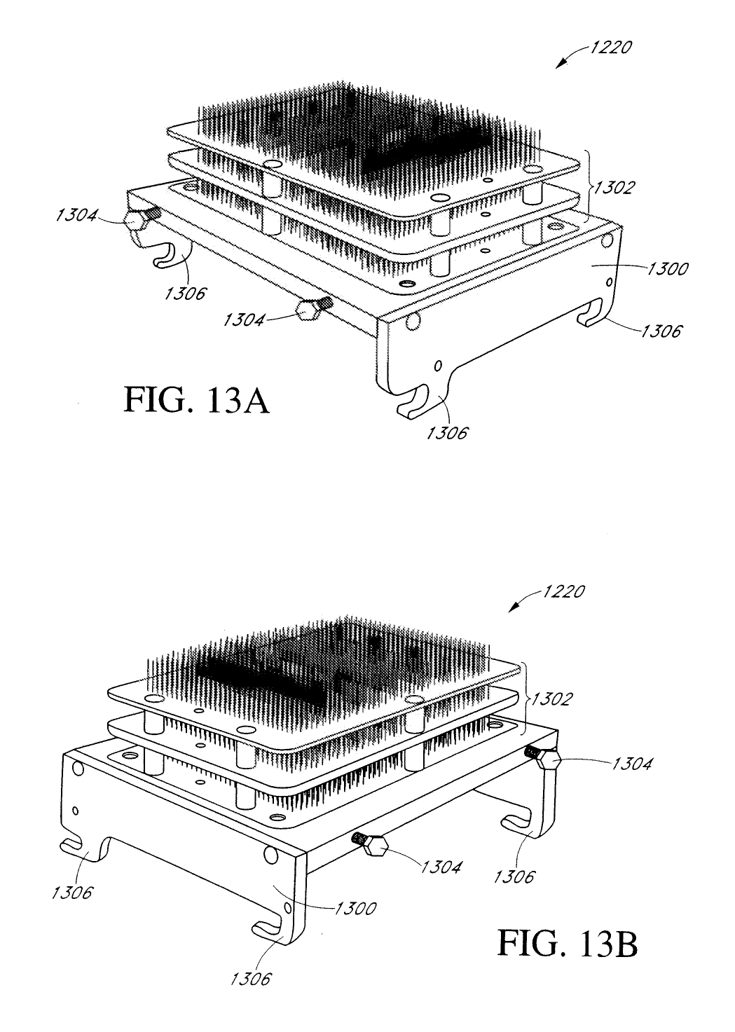

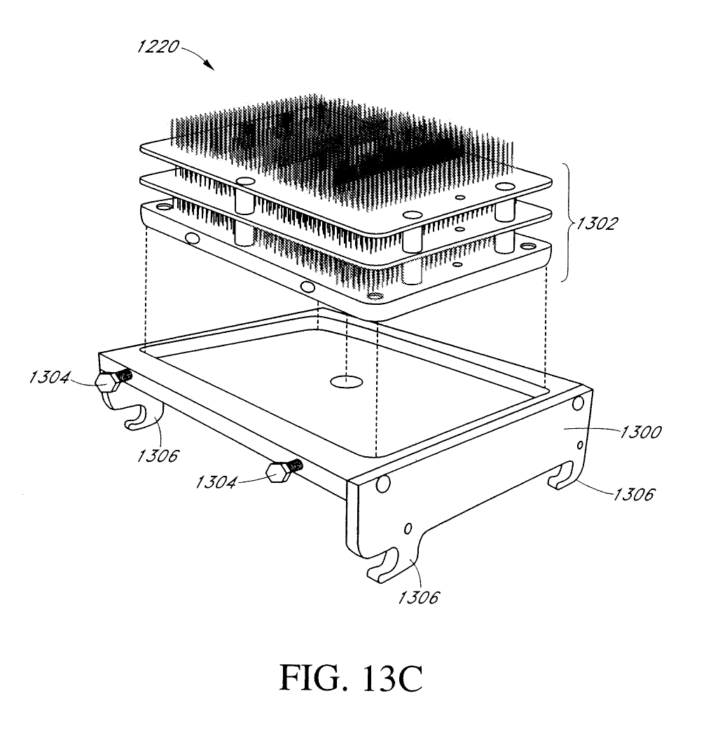

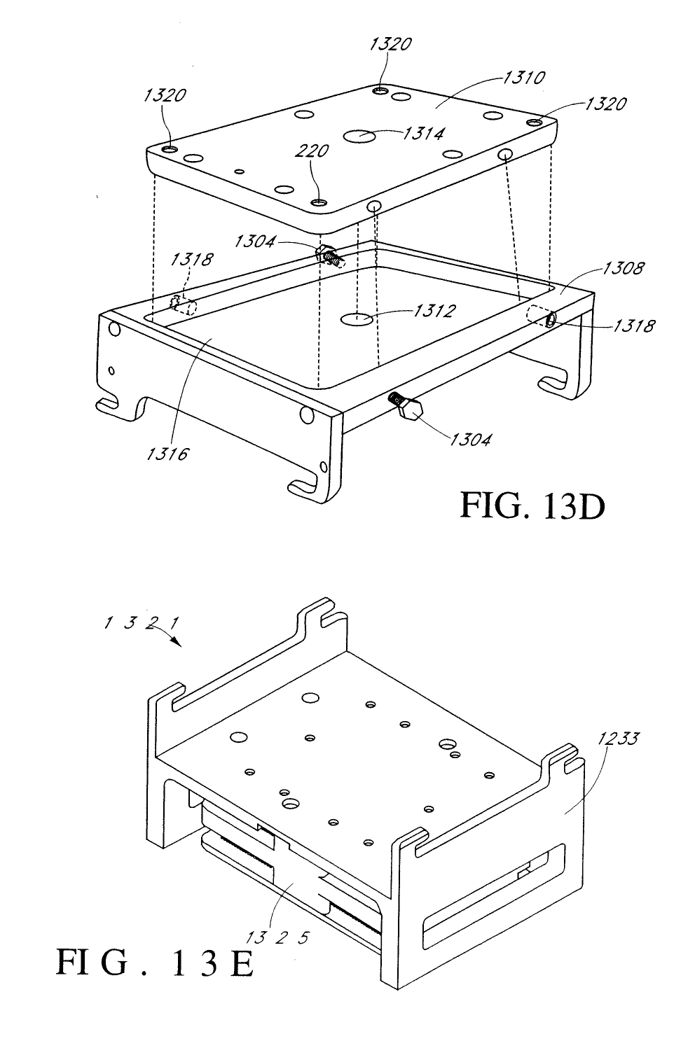

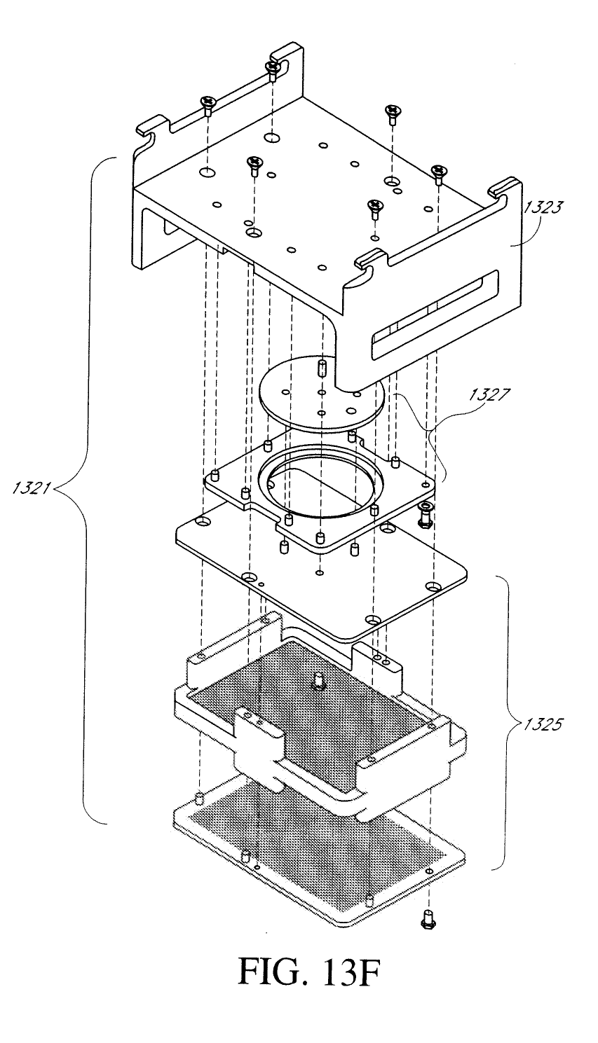

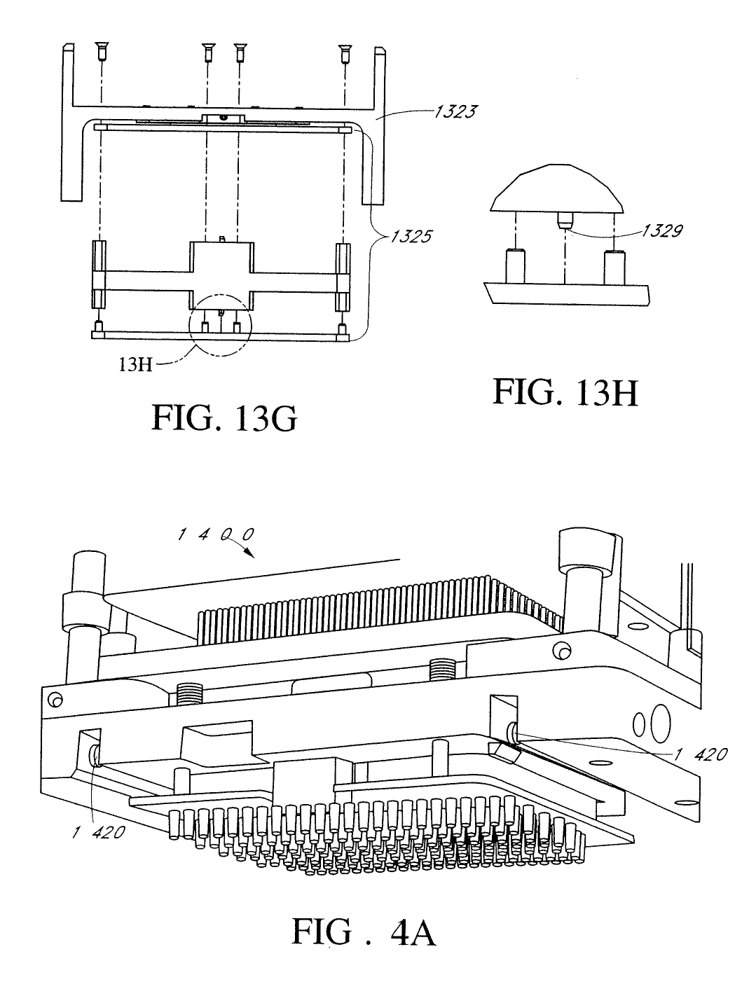

FIGS. 13A-C schematically show pin tool 1220 from various perspective views according to one embodiment. As shown, pin tool 1220 includes support structure 1300 and pin tool head 1302. Pin tool head 1302 is removably attached to support structure 1300 by set screws 1304. Pin tool heads typically include a mounting plate and one or more floating fixtures or plates. As also shown, support structure 1300 also includes hooks 1306, which removably attach support structure 1300 to another component of the fluid transfer device, such as the chassis of the fluid transfer device, which is described further below. Pin tool head 1302 includes 1536 pins in a 32×48 array that has a footprint corresponding to 1536-well micro-well plate. The pin tool heads of the assaying components of the invention optionally include other array configurations and/or numbers of pins to transfer fluid samples to and/or remove such fluid samples from selected multi-well containers or support surfaces. In certain embodiments, pin tool heads of the systems described herein include numbers of pins that correspond to the number of wells in various standard multi-well plates, such as those having, e.g., 6, 12, 24, 48, 96, 192, 384, 768, 1536, 3456, 9600, or more wells. A wide variety of pin tools and pins are optionally used in the systems of the invention and some are commercially available from sources, such as V&P Scientific, Inc. (San Diego, Calif., USA), Beckman Coulter, Inc. (Fullerton, Calif., USA), Perkin Elmer Life Sciences (Boston, Mass., USA), and the like. Pins, for example, can be of varied lengths selected, e.g., according to the depth of the containers to be accessed. Pins can also have various cross-sectional dimensions (e.g., diameters, etc.) and be slotted, solid, etc. or otherwise varied according to the fluid volumes to be transferred. Pins can also be uncoated, or coated with, e.g., hydrophobic or lipophobic coating to provide additional control over the transfer of various types of solutions (e.g., organic or aqueous solutions).

In some embodiments, the pin tools of the assaying components described herein include low profile rotational adjustment features or mounts. Conventional pin tools lack an intrinsic mechanism to adjust for the rotational axis of the pin tool. Instead, conventional devices are typically coupled to a separate rotational mount. An advantage of these pin tools is that a low profile rotational adjustment is generally built into the pin tools themselves, thereby eliminating the need for separate rotational mounts. This is schematically illustrated in FIG. 13D, which shows pin tool support structure 1308 and top plate 1310 of a pin tool head (floating plates and pins are not shown) from an exploded perspective view according to one embodiment. Pin tool support structure 1308 and top plate 1310 each include center holes 1312 and 1314, respectively, which align with one another when top plate 1310 is positioned in top plate inset region 1316 of pin tool support structure 1308. Center holes 1312 and 1314 are each typically threaded to receive a center screw (not shown), which can be used to adjust the rotational axis of an attached pin tool head. Other functionally equivalent components aside from center screws (e.g., posts, ball and socket joints, etc.) can also be adapted for use as rotational adjustment features of the pin tools of the invention. In the embodiment shown in FIG. 13D, pin tool support structure 1308 also includes spring tension devices 1318 (e.g., spring ball sockets, etc.) opposed by set screws 1304 to rotate the pin tool head about the center screw. In other embodiments, support structures include only set screws 1304. This is shown, for example, in FIGS. 13A-C. Holes 1320 are typically included to attach top plate stand-off components (e.g., flexed metal or polymeric strips, springs, elastomers, etc.) that resiliently couple a pin tool head to pin tool support structure 1308. Optionally, top plate stand-off components are not included or are attached to pin tool support structure 1308.

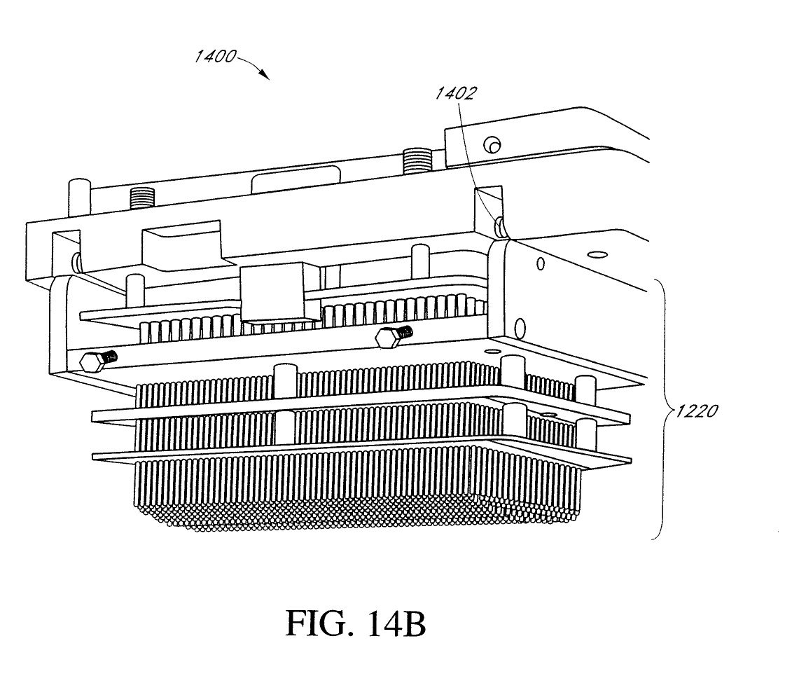

Pin tools typically removably attach to other components of the fluid transfer devices of the assaying systems by various attachment features, including the hook mounts described above. In certain embodiments, for example, pin tools removably attach a chassis of a pressure-based fluid transfer device (e.g., a pipetting system, etc.) to afford the user the option of using either a pin tool or pipettes to transfer fluids between various types of containers and/or supports. FIG. 14A schematically shows a chassis of a fluid transfer device that includes such a pipetting system. As shown, chassis 1400 includes horizontal posts 1402 (two are not within view) to which hooks 1306 of pin tool 1220 are capable of being attached. FIG. 14B schematically depicts pin tool 1220 attached to chassis 1400 via horizontal posts 1402. When pin tools are not attached to fluid transfer device chassis, they are optionally disposed in a docking station. In certain embodiments, for example, wash stations can also function as docking stations for pin tools. Docking and wash stations are described in greater detail below. In some embodiments, fluid transfer devices do not include pipetting systems in addition to the capability of using pin tools to effect fluid transfer. In these embodiments, at least pin tool support structures are optionally manufactured as non-removable components of fluid transfer devices.

To further illustrate, FIGS. 13 E and F schematically illustrate another exemplary pin tool according to one embodiment of the invention. More specifically, FIG. 13E schematically shows pin tool 1321 from a perspective view, while FIGS. 13 F and G schematically depict pin tool 1321 from exploded perspective and exploded front views, respectively. As shown, pin tool 1321 includes support structure 1323 and pin tool head 1325 (pins not shown). Pin tool 1321 also includes rotational adjustment feature 1327 (shown as a rotation stage and as a rotation stage capture block). FIG. 13H schematically shows an interface between components of pin tool head 1325 from pin tool 1321 from a detailed front view. The interface includes dowel pin 1329, which is received by an opposing hole (not within view) when pin tool head 1325 is assembled.

The fluid transfer devices of the assaying components of the systems of the invention typically include robotic translation systems (e.g., X-Y-Z translations systems, etc.) that move pin tools relative other components of the system. In certain embodiments, for example, a fluid transfer device lowers a pin tool such that the pins contact fluidic samples in a multi-well sample compound plate. The fluid transfer device then typically withdraws from the compound plate such that fluid adheres to the pins of the pin tool and translocates the pin tool such that the fluidic samples volumes adhered to the pins are dispensed into corresponding wells in a multi-well sample assay plate for analysis, e.g., excitation by the electromagnetic radiation from the electromagnetic radiation source and detection of sample electromagnetic radiation from the assay plate by the detector. Robotic translations systems are typically operably connected to controllers of the assay systems, which controller generally includes one or more computers or other logic devices having system software that directs the operation of the translation systems. Controllers are described in greater detail below.

ii. Sample Assaying Regions, Container Positioning Devices, and Fluid Transfer Probe Washing and Drying Stations

The sample assaying regions of the assaying components of the systems of the invention are configured to receive source electromagnetic radiation from the electromagnetic radiation source. In certain embodiments, sample assaying regions also include container positioning devices that position containers relative to the fluid transfer device and/or the detector. Sample assaying regions optionally further include fluid transfer probe washing stations to wash fluid transfer probes before and/or after selected fluid transfer processes, and fluid transfer probe drying stations (e.g., blotting stations, vacuum drying stations, etc.) to dry fluid transfer probes as desired.

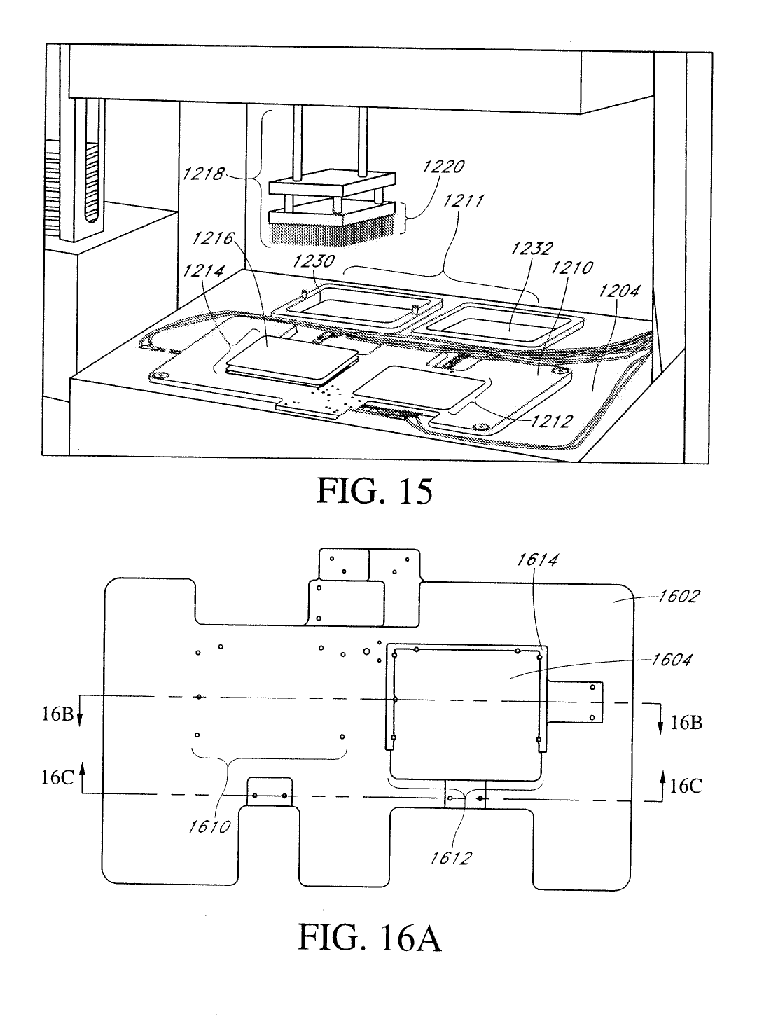

FIG. 15 schematically shows sample assaying region 1204 from a perspective view according to one embodiment. As shown, sample assaying region 1204 includes container positioning device 1210, which includes container stations 1212 and 1214 that are each structured to position containers relative to fluid transfer device 1218. In some embodiments, container stations 1212 and 1214 are structured to position multi-well plates. In compound profiling applications, for example, container station 1212 is typically utilized to position a multi-well plate containing sample compounds and container station 1214 is typically utilized to position an assay multi-well plate into which compounds are transferred from the sample compound multi-well plate positioned in container station 1212 using fluid transfer device 1218. As also shown in this embodiment, sample assaying region 1204 additionally includes fluid transfer probe washing station 1211. Certain assay protocols include washing pin tool 1220 in one or both wash reservoirs 1230 and 1232 before and/or after performing a particular transfer step. Optionally, wash reservoir 1230 is also used as a docking station to position pin tool 1220 when it is detached from the chassis of fluid transfer device 1218. In certain embodiments, fluid transfer probe washing stations are not included in the assaying systems of the invention or are located in a region other than sample assaying region 1204. In some embodiments, for example, one or both of reservoirs 1230 and 1232 are replaced by fluid transfer probe blotting stations or vacuum drying stations, which effect the removal of fluids that adhere to the pins of pin tool 1220. Each of these system components is described in greater detail below.

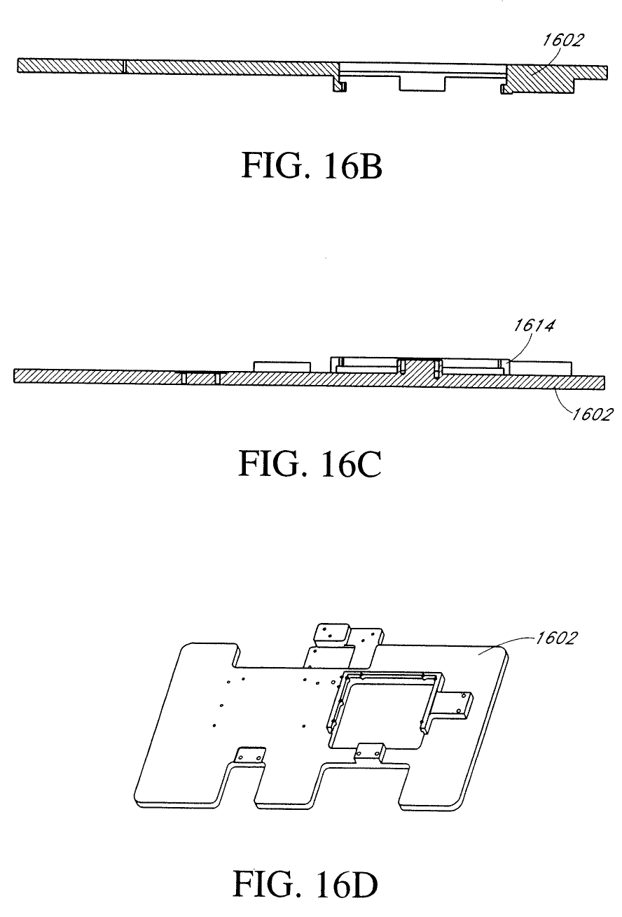

In certain embodiments, the sample assaying regions of the assaying components of the systems described herein include container positioning devices, e.g., to position sample containers relative to fluid transfer devices. The container positioning devices of the invention generally include multiple container stations, e.g., to position multiple containers for fluid transfer when performing a given assay. In some embodiments, at least two of the container stations are tiered, that is, disposed at different levels. In systems that include robotic handlers, tiered container stations have the advantage of allowing a robotic handler to access and handle (e.g., grasp and re-locate) a first container positioned at one tiered container station without contacting a second container positioned at another tiered container station. This is further illustrated in, e.g., FIGS. 16A-D. In particular, FIG. 16A schematically shows support structure 1602 of container positioning device 1600 from a top view. As shown, support structure 1602 includes container station 1610 and container station 1612. Container station 1612 includes orifice 1604 disposed through support structure 1602, as described above. In addition, container station 1612 further includes tier structure 1614 disposed around a portion of orifice 1604. Tier structure 1614 positions containers at a different level in container station 1612 than those positioned in container station 1610. FIGS. 16 B and C schematically depict cross-sectional side views of support structure 1602 shown in FIG. 16A along sections 16B and 16C, respectively. To further illustrate, FIG. 16D schematically illustrates support structure 1602 from a top perspective view.

The container stations of the container positioning devices of the invention also optionally include heating elements (e.g., external to or integral with the container stations) to regulate temperature in the container or on the other support, e.g., when an assay is performed in the system. Suitable heating elements that can be adapted for use in the systems of the invention are generally known to persons of skill in the art and are readily available from various commercial sources. Heating elements are typically operably connected to system controllers, which control operation of the elements.

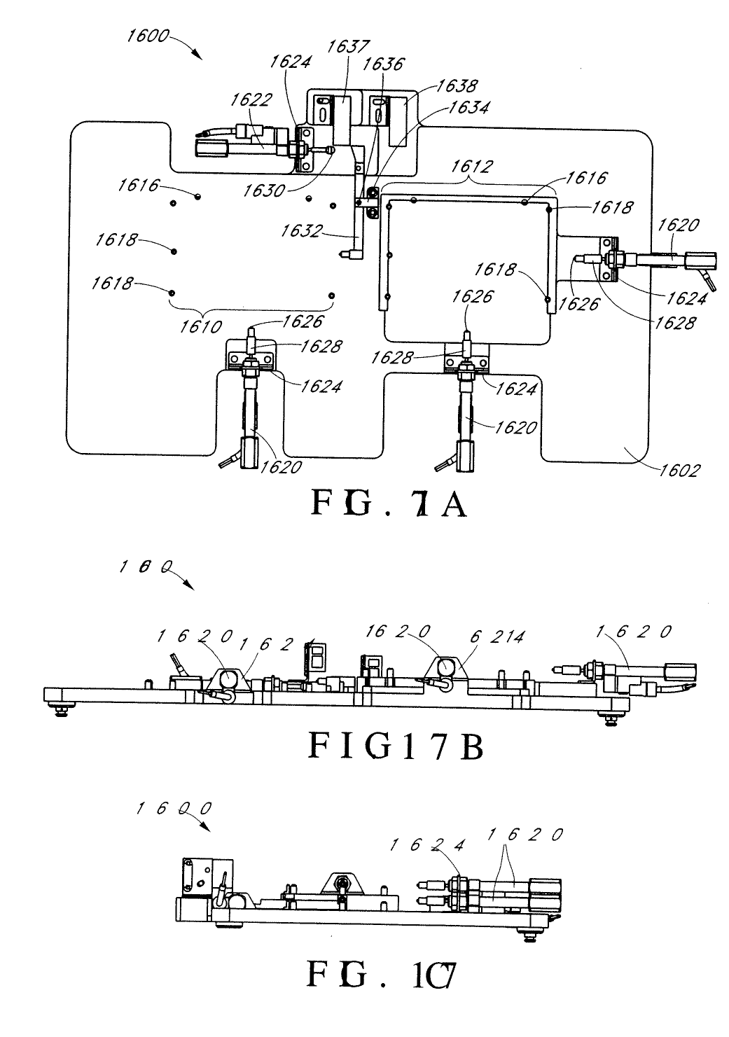

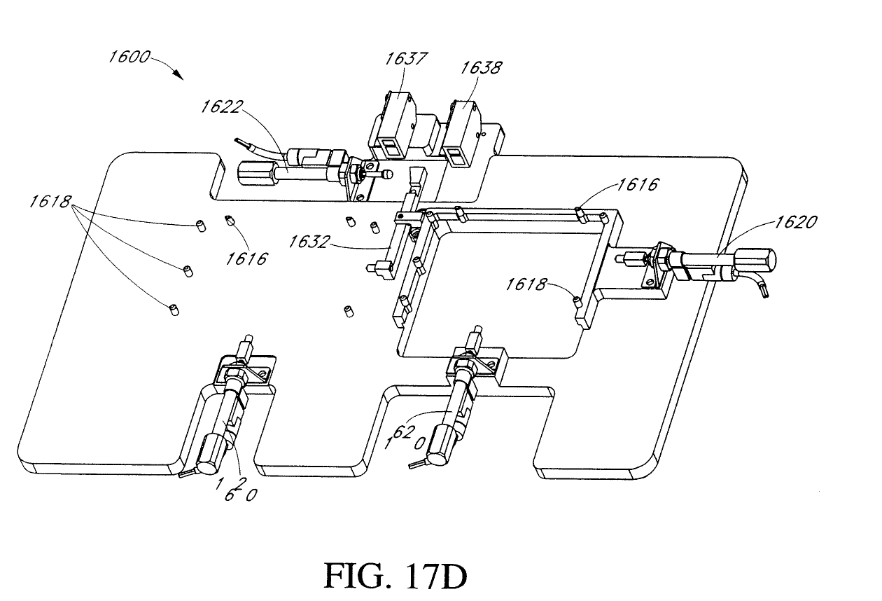

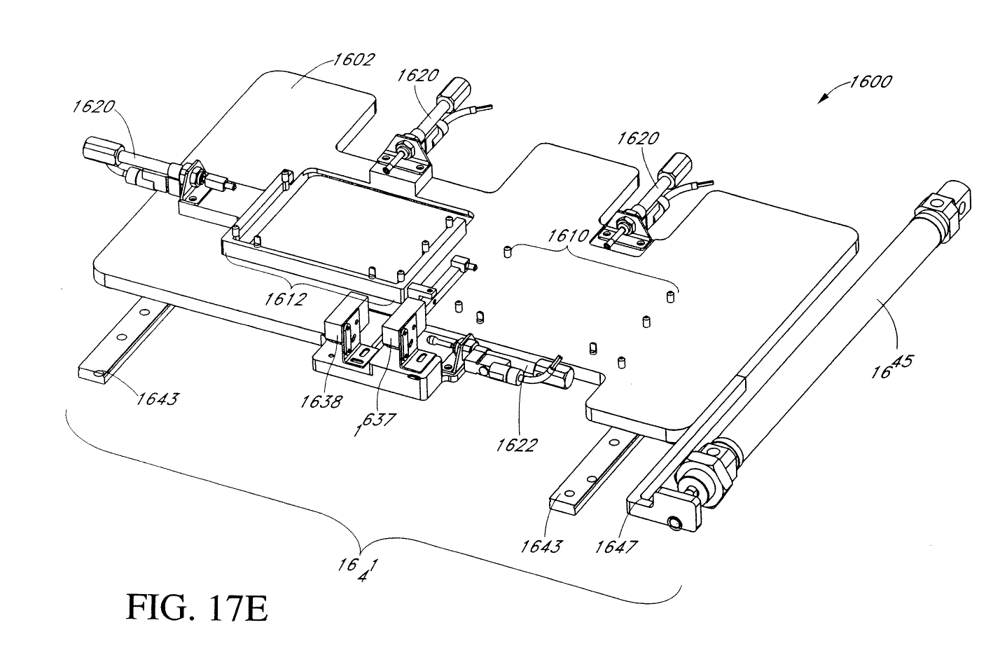

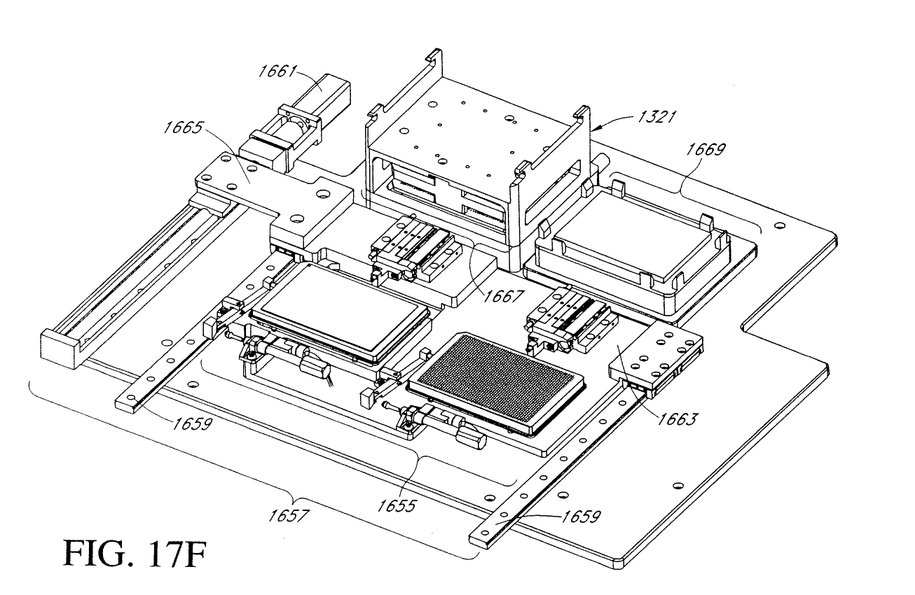

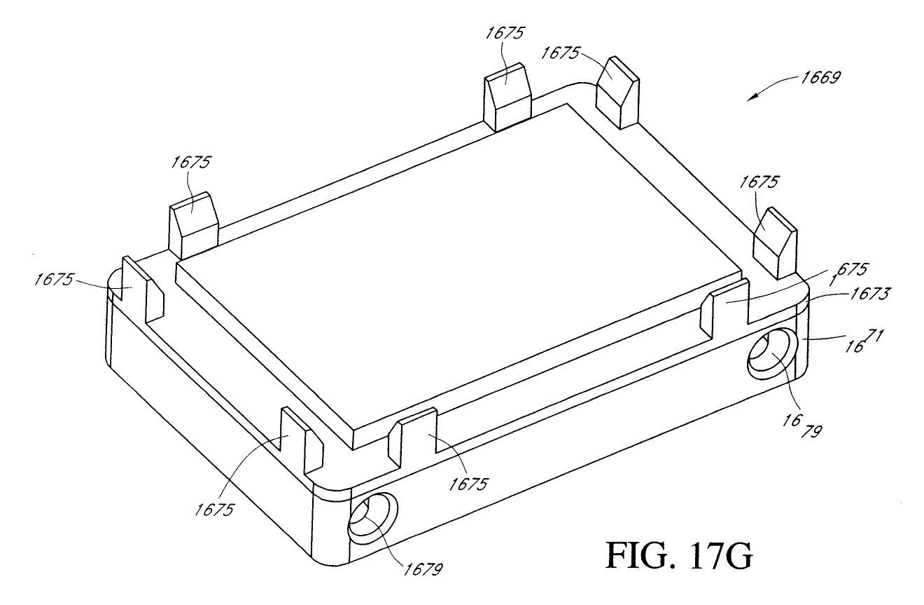

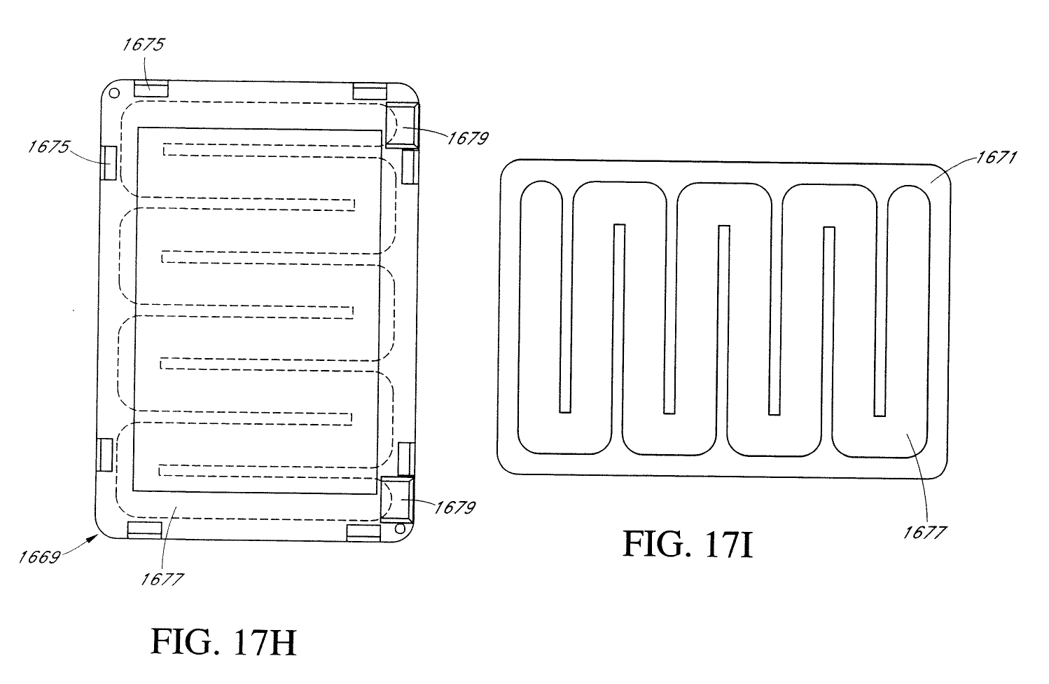

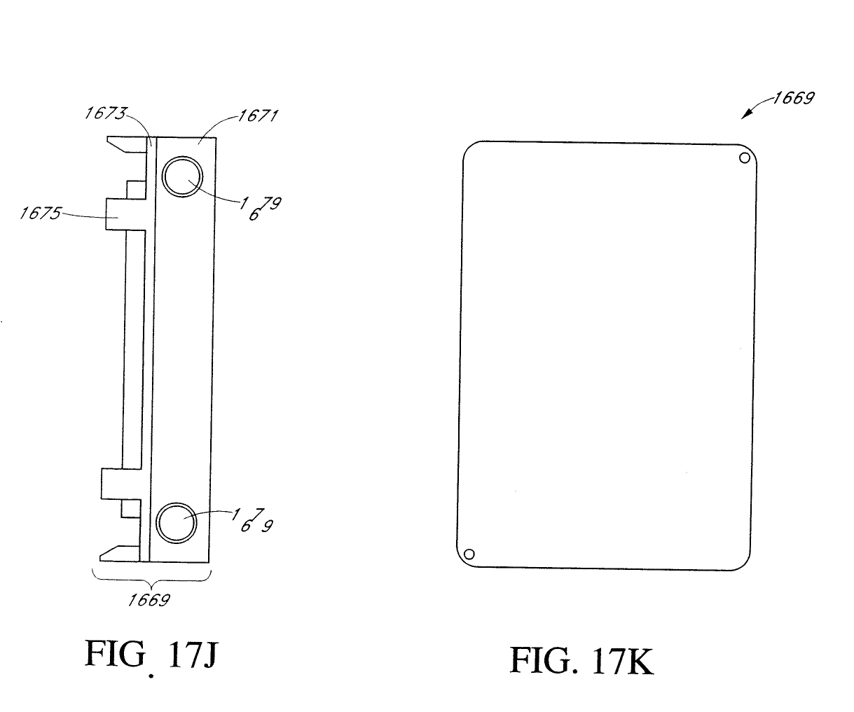

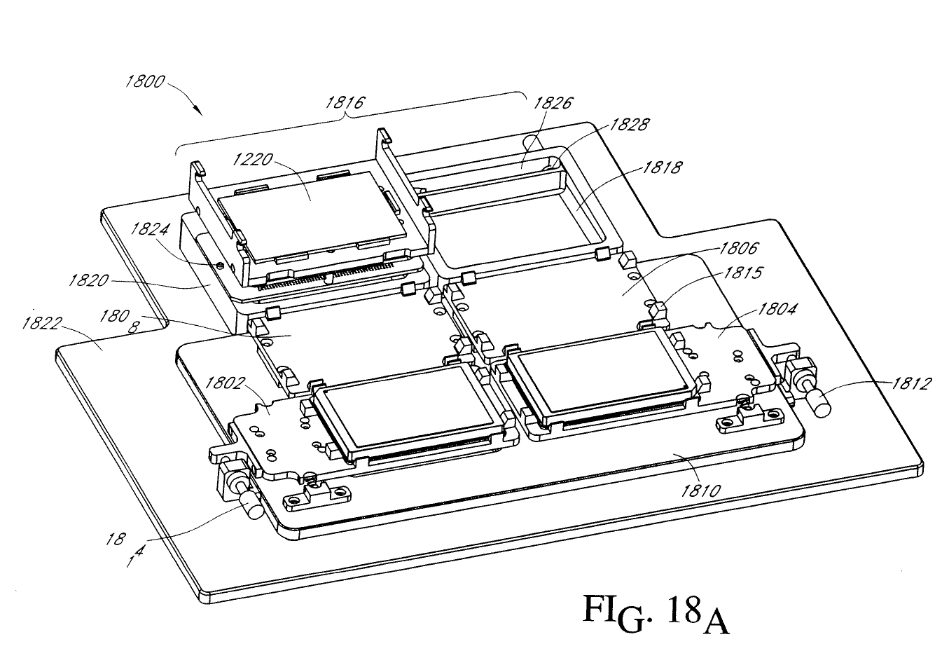

Container positioning devices also generally include alignment members that are positioned to contact surfaces of containers when the containers are positioned in the container stations such that the containers align with the fluid transfer device. In addition, these container positioning devices also typically include pushers that push the containers into contact with the alignment members when the containers are positioned in the container stations. Embodiments of these aspects of container positioning devices are illustrated in FIGS. 17A-D. More specifically, FIG. 17A schematically shows container positioning device 1600 from a top view. As shown, container positioning device 1600 includes alignment members 1616 (shown as trimmed face pins) and alignment members 1618 (shown as pins), which align with inner surfaces of standard multi-well plates positioned in container stations 1610 and 1612. As also shown, container positioning device 1600 further includes pneumatically-driven pushers 1620 and 1622 (e.g., air cylinders or the like), which effect container positioning relative to alignment members 1616 and 1618. Pushers 1620 and 1622 are mounted to support structure 1602 via pusher mounts 1624 and are operably connected to pressure sources (not shown). Pushers 1620 include spring plungers 1626 and plunger posts 1628. Pusher 1622 includes knob 1630 that contacts lever arm 1632 to push lever arm 1632 into contact with a container. Lever arm 1632 is mounted to support structure 1602 via pin capture block 1634 and lever shaft 1636. As also shown in FIG. 17A, container positioning device 1600 also includes laser assemblies 1637 and 1638 for detecting the presence of containers in container stations 1610 and 1612, respectively. FIGS. 17 B and C schematically show container positioning device 1600 from side elevational views. In addition, FIG. 17D schematically illustrates container positioning device 1600 from a perspective view.

To further illustrate aspects of container positioning devices, FIG. 17E schematically shows a perspective view of container positioning device 1600 of FIG. 17A mounted on translational mechanism 1641. When container positioning devices are included in system components such as assaying component 1200 schematically shown in FIG. 12, translational mechanisms are optionally included such that container positioning devices can be translocated along at least one translational axis, e.g., to facilitate access to multi-well containers positioned in the container positioning devices by a user, a robotic gripping device, and/or the like. In the embodiment shown, translational mechanism 1641 includes rails or tracks 1643 on which container positioning device 1600 is mounted and along which container positioning device 1600 slides. In addition, actuator 1645 (e.g., an air cylinder, motor, etc.) is operably connected to support structure 1602 of container positioning device 1600 via bracket 1647. Actuator 1645, which is generally operably connected to a controller, effects translocation of container positioning device 1600 along tracks 1643.

To further illustrate additional aspects of container positioning devices, FIG. 17F schematically shows a perspective view of sample assaying region 1663, which includes container positioning device 1655 mounted on translational mechanism 1657. As referred to above, translational mechanisms are optionally included so that container positioning devices can be translocated along at least one translational axis. In the embodiment shown, translational mechanism 1657 includes rails or tracks 1659 on which container positioning device 1655 is mounted and along which container positioning device 1655 slides. In addition, actuator 1661 (e.g., an air cylinder, motor, etc.) is operably connected to support structure 1663 of container positioning device 1655 via bracket 1665. Actuator 1661, which is generally operably connected to a controller, effects translocation of container positioning device 1655 along tracks 1659.

As also shown in FIG. 17F, sample assaying region 1663 also includes wash reservoir 1667 and thermal modulation nest 1669 according certain illustrative embodiments. Wash reservoirs and stations are also described further below. Thermal modulation nests are typically used to regulate temperatures in containers (e.g., compound plates, assay plates, etc.). To further illustrate, FIGS. 17G-K schematically depict various aspects of thermal modulation nest 1669. More specifically, FIG. 17G schematically depicts thermal modulation nest 1669 from a perspective view, FIG. 17H schematically shows thermal modulation nest 1669 from a transparent top view, FIG. 17I schematically shows bottom plate 1671 of thermal modulation nest 1669 from a top view, FIG. 17J schematically illustrates thermal modulation nest 1669 from a front view, and FIG. 17 schematically depicts thermal modulation nest 1669 from a bottom view. As shown, thermal modulation nest 1669 includes top plate 1673 and bottom plate 1671, which are generally attached (e.g., welded, bonded, adhered, etc.) to one another in an assembled device. Although other materials are optionally utilized, top plate 1673 and bottom plate 1671 are both fabricated from stainless steel in certain embodiments. Top plate 1673 typically includes nest features 1675 formed on a surface (e.g., via machining, molding, etc.), which are used to align containers on thermal modulation nest 1669. Bottom plate 1671 includes channel 1677 (shown with a serpentine flow path), which communicates with orifices 1679. Channels and orifices are typically formed by machining or other processes known to persons of skill in the art.

During operation, hoses are generally attached to orifices 1679 and heated or cooled fluids are circulated through the hoses and channel 1677 via orifices 1679, e.g., to regulate temperatures in a container (e.g., a control plate or boat, etc.) disposed on thermal modulation nest 1669. In certain embodiments, for example, the hoses are operably connected to a recirculated chiller unit (e.g., a NESLAB RTE-7 available from Thermo Electron Corporation (Newington, N.H., USA)). In these embodiments, the chiller unit typically cools a 50/50 ethylene-glycol and water mixture to 4° C. and circulates the fluid through thermal modulation nest 1669. Typically, a drip tray or the like is positioned underneath thermal modulation nest 1669 to catch condensate that forms on thermal modulation nest 1669. Containers positioned on thermal modulation nest 1669 are typically accessible by the pin tools described herein.

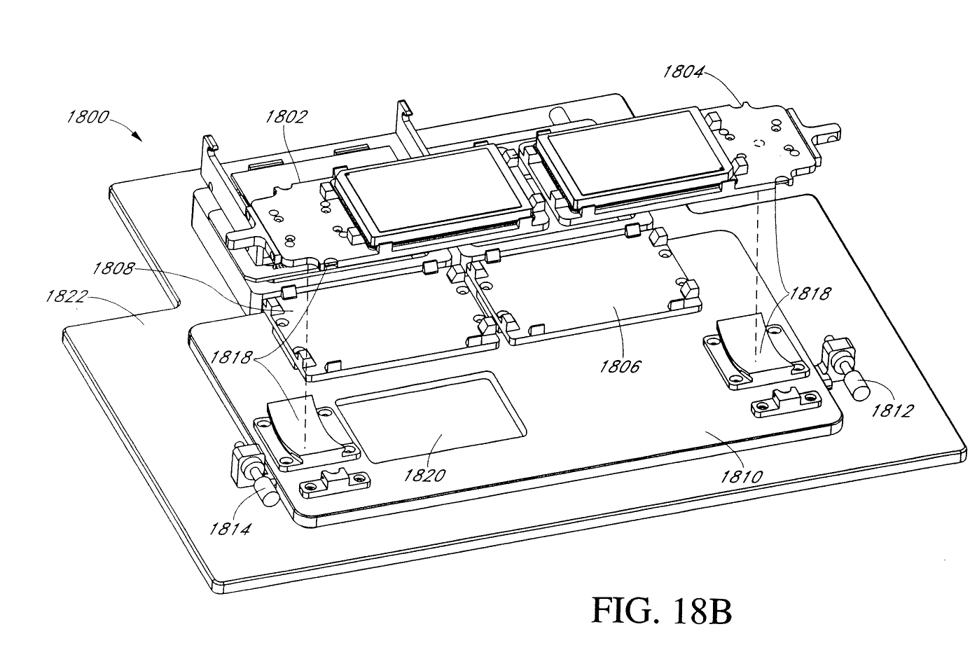

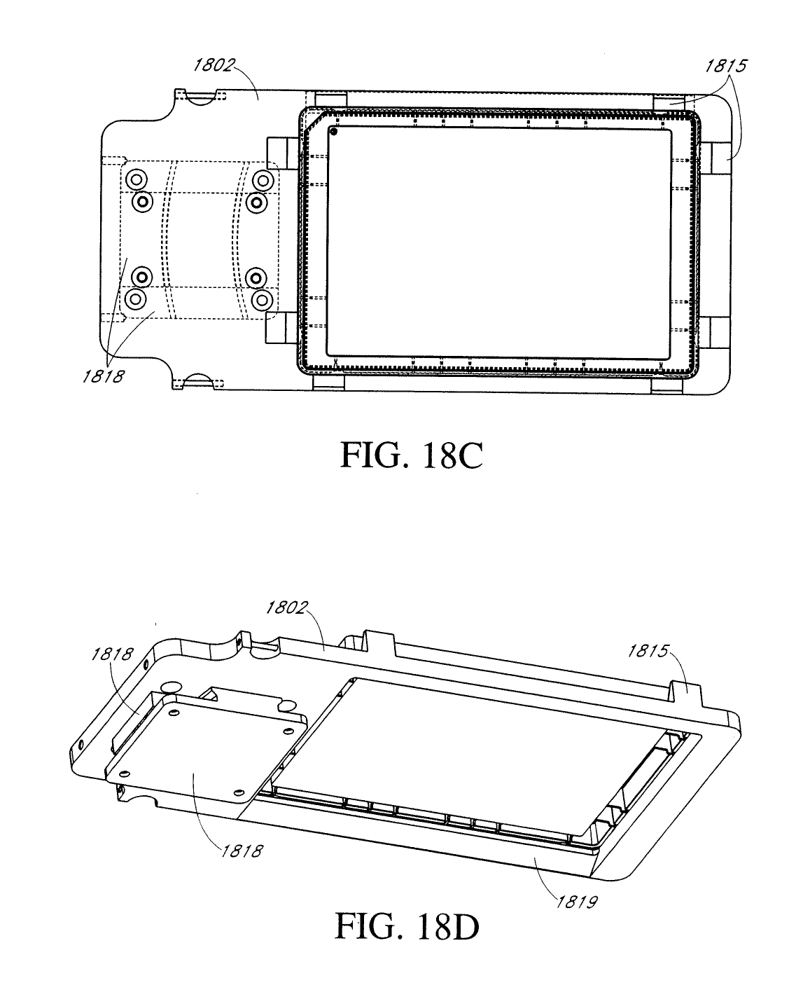

Container positioning devices also include other embodiments. For example, FIG. 18A schematically shows container positioning device 1800 from a perspective view. As shown, container positioning device 1800 includes nests 1802, 1804, 1806, and 1808 in which multi-well containers can be placed to position the containers relative to the fluid transfer device. Nests 1802, 1804, 1806, and 1808 are typically precisely fabricated (e.g., machined, molded, etc.) such that sample plates fit tightly (i.e., substantially without room for lateral movement, etc.) into nests 1802, 1804, 1806, and 1808. Component fabrication is described further below. As shown, nests 1802, 1804, 1806, and 1808 each include multiple alignment members 1815 that include angled surfaces that are configured to direct multi-well containers into nests 1802, 1804, 1806, and 1808, respectively, when such containers are placed into those nests. Nests 1802 and 1804 are fabricated to rotate about the centers of plates positioned in those nests so that plate positions can be adjusted to align with the pin tool of the fluid transfer device. This eliminates the need to include a corresponding rotational adjustment in, e.g., the pin tool and/or fluid transfer device chassis. However, in some embodiments, these other rotational adjustments are also included for additional control over the alignment of the pin tool and plates.

FIG. 18B schematically shows positioning device 1800 of FIG. 18A from a partially exploded perspective view. As shown, nest 1802 and 1804 rotate about rotational coupling components 1818 (shown as a carriage and base that mate via a dovetail joint) that mate with or otherwise contact both the particular nest and top tier support structure component 1810 of positioning device 1800, which are typically disposed proximal to an end of the particular nest. Rotational coupling components 1818 are typically fabricated from stainless steel with a thin (e.g., 0.002 inches thick) brass, TEFLON™, or other shim inserted between the two pieces to provide a bearing surface. Other rotational couplings, which are generally known to persons of skill in the art, are also optionally utilized. The rotational positions of nests 1802 and 1804 are individually adjusted using set screws 1814 and 1812, respectively, or other functionally equivalent rotational adjustment features. Springs 1815 provide counteracting tension to set screws 1814 and 1812 to maintain the selected rotational position of nests 1802 and 1804. In addition, nest 1802 includes orifice or cutout 1820 so that when a container is positioned over the orifice 1820, the container can receive electromagnetic radiation from an electromagnetic source and/or the detector can receive electromagnetic radiation from the container through orifice (e.g., via an optical system, etc.). Additional details relating to container positioning devices which are optionally adapted for use in assaying components or other work stations of the systems of the present invention are described in, e.g., International Publication No. WO 01/96880, entitled “AUTOMATED PRECISION OBJECT HOLDER,” filed Jun. 15, 2001 by Mainquist et al., U.S. patent application Ser. No. 10/911,238, entitled “MULTI-WELL CONTAINER POSITIONING DEVICES AND RELATED SYSTEMS AND METHODS,” filed Aug. 3, 2004 by Evans, and U.S. Provisional Patent Application No. 60/645,502, entitled “MULTI-WELL CONTAINER POSITIONING DEVICES, SYSTEMS, COMPUTER PROGRAM PRODUCTS, AND METHODS,” filed Jan. 19, 2005 by Chang et al., which are incorporated by reference.

To further illustrate the invention, FIG. 18C schematically shows a partially transparent top view of a portion of nest 1802 of positioning device 1800. The relative orientation of rotational coupling components 1818 is shown. This is further depicted in FIG. 18D, which schematically shows nest 1802 from a bottom perspective view. As shown, edge 1819 includes an angled cut surface (e.g., at approximately 45°) to allow, e.g., electromagnetic radiation from an excitation laser or other electromagnetic radiation source to be incident on any selected well of a given multi-well container without being obstructed the nest structure. These angled edges are also typically included in other container stations having orifices as described herein.

Nests 1806 and 1808 are optionally used to position additional sample plates. In some embodiments, at least one of nests 1806 and 1808 is used as a fluid transfer probe or pin tool blotting station to remove adherent fluid from the probe before or after a fluid transfer step is performed. In these embodiments, blotting paper (not shown) is placed in, e.g., nest 1806 and pin tool 1220 is contacted with the paper such that adherent fluid is blotted, wicked, or otherwise removed from the pins of pin tool 1220. Various types of blotting paper including, e.g., lint-free blotting paper, etc. are commercially available from many different suppliers, such as V&P Scientific, Inc. (San Diego, Calif., USA) or the like.

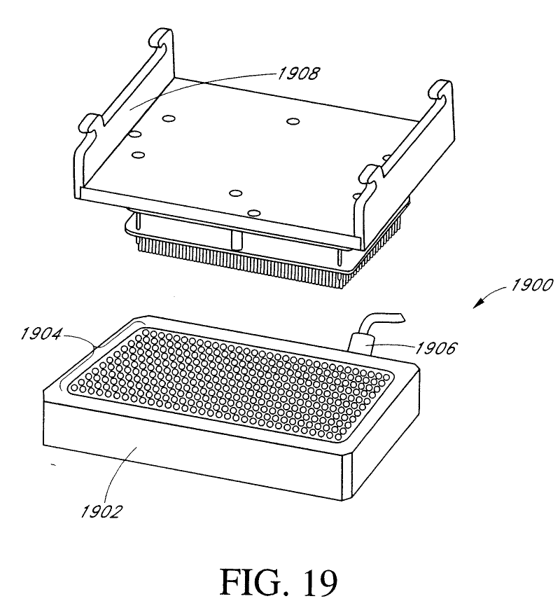

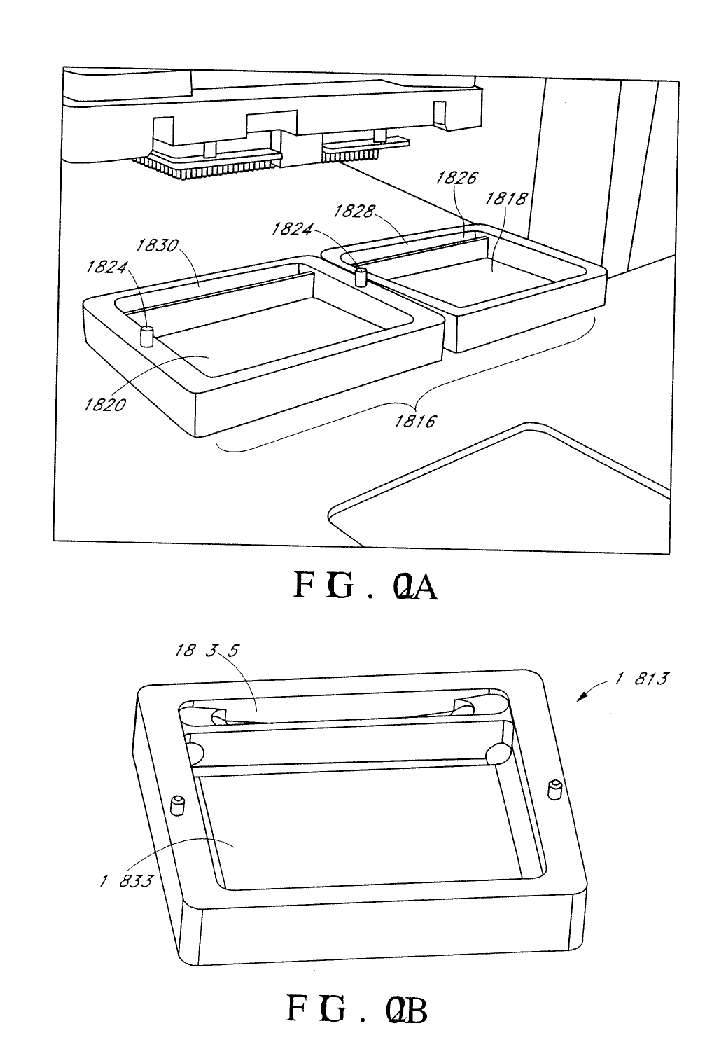

In certain embodiments, the assaying components further includes a fluid transfer probe or pin tool vacuum drying station that removes adherent fluid from the pins under an applied vacuum when the pin tool is disposed proximal to the vacuum drying station. Optionally, such a vacuum drying station replaces, e.g., nest 1806 and/or nest 1808 or is positioned at another location that is either internal or external to the assaying component. An exemplary fluid transfer probe vacuum drying station is schematically depicted in FIG. 19. As shown, vacuum drying station 1900 includes vacuum drying station body structure 1902, which includes array of holes 1904 through which vacuum is applied to effect the removal of adherent fluid from the pins of pin tool 1908 when the pins are positioned proximal to array of holes 1904 by the fluid transfer device. In some embodiments, vacuum holes are arrayed to have a footprint that corresponds to the pins of the particular pin tool being utilized (e.g., a one-to-one correspondence). In other embodiments, a one-to-one correspondence between the number of vacuum holes and the number of pins is not present. For example, if there are fewer holes in the particular array than in the pin tool, then the applied vacuum is typically increased so that a given hole can remove adherent fluid from multiple pins. Vacuum is typically applied via a vacuum line operably connected to vacuum port 1906.

As additionally shown in FIG. 18A, container positioning device 1800 also includes fluid transfer probe washing station 1816, which includes wash reservoirs 1818 and 1820 (e.g., recirculation troughs or baths, etc.) disposed on bottom tier support structure component 1822 of container positioning device 1800. Wash reservoirs 1818 and 1820 are generally filled with a wash solvent such as dimethyl sulfoxide (DMSO), ethanol, methanol, water, or the like and are used to wash pin tool 1220. For example, one washing or cleaning protocol includes filling wash reservoir 1820 with DMSO and filling wash reservoir 1818 with ethanol (or methanol). In this cleaning protocol, after compounds are transferred from a compound plate to an assay plate, the pins of pin tool 1220 are first dipped into the DMSO bath, followed by being dipped into the ethanol (or methanol) bath. In embodiments that include the blotting stations described above, the pins are then typically contacted with the blotting paper to remove the wash solvent. As one alternative to this wash protocol, after compound transfer, the pins are blotted before being dipped into wash reservoirs 1820 and 1818, as described above. As also shown, fluid transfer probe washing station 1816 also includes overflow reservoir 1826 that fluidly communicates wash reservoir 1818 by reservoir divider 1828, which is disposed below the level of the openings to wash reservoir 1818 and overflow reservoir 1826. Overflow reservoir 1826 prevents wash solvent from overflowing from wash reservoir 1818, e.g., onto other components of the assaying component. Although not within view in FIG. 18A, an overflow reservoir also fluidly communicates with wash reservoir 1820. This is illustrated in FIG. 20A, which schematically shows fluid transfer probe washing station 1816 from a perspective view. As shown, overflow reservoir 1830 fluidly communicates with wash reservoir 1820. To further illustrate another exemplary embodiment, FIG. 20B shows fluid transfer probe washing station 1831, which includes wash reservoir 1833 and overflow reservoir 1835.

Optionally, at least one of wash reservoirs 1818 and 1820 is used as a docking station for pin tool 1220 when it is not attached to the chassis of the fluid transfer device. As shown in FIG. 18A, for example, wash reservoir 1820 includes first alignment features 1824 (e.g., pins, etc.) (one not within view) and a floating plate of pin tool 1220 includes second alignment features (e.g., holes, etc.) (one not within view) that correspond to first alignment features 1824. For example, when the fluid transfer device dips pin tool 1220 into wash reservoir 1820, first alignment features 1824 and the corresponding second alignment features mate with one another to align pin tool 1220 relative to wash reservoir 1820 such that the fluid transfer device chassis can detach from pin tool 1220. These alignment features also align pin tool 1220 and wash reservoir 1820 when the pins are washed, e.g., according to a wash protocol described herein.

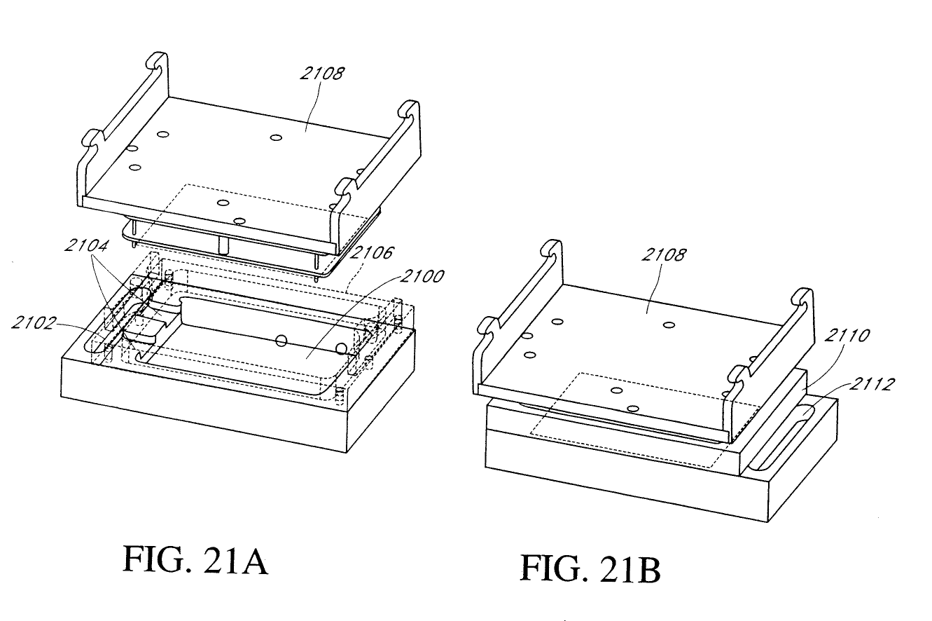

To illustrate another embodiment, FIG. 21A schematically shows wash reservoir 2100 from a perspective view. As shown, wash reservoir 2100 fluidly communicates with overflow reservoir 2102 via overflow channels 2104. FIG. 21A also shows a transparent perspective view of non-pressure-based fluid transfer probe mount 2106 disposed around wash reservoir 2100. Non-pressure-based fluid transfer probe mount 2106 is optionally utilized to mount or position non-pressure-based fluid transfer probe 2108 relative to wash reservoir 2100 when non-pressure-based fluid transfer probe 2108 is washed and/or when non-pressure-based fluid transfer probe 2108 is separated from a chassis of the fluid transfer device. In addition, FIG. 21B schematically shows non-pressure-based fluid transfer probe 2108 positioned or mounted on non-pressure-based fluid transfer probe mount 2110 from a perspective view. As shown, the wash reservoir (not within view) and overflow reservoir 2112 mirror the orientation of wash reservoir 2100 and non-pressure-based fluid transfer probe mount 2106 depicted in FIG. 21A.

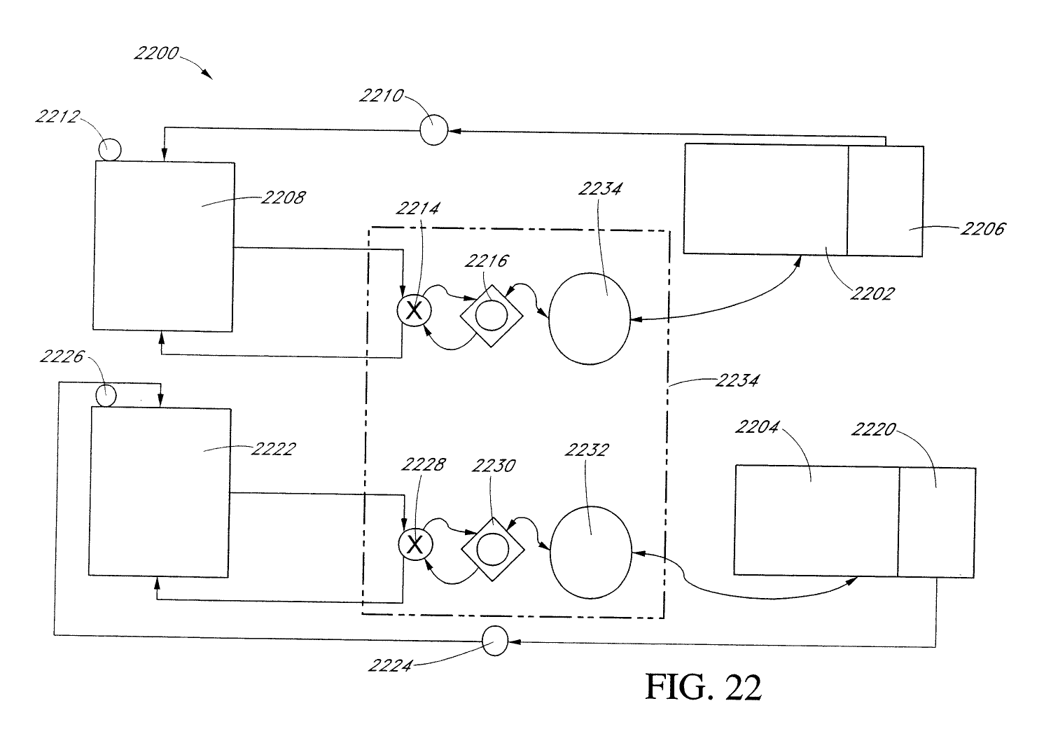

FIG. 22 is a block diagram showing representative fluid transfer probe washing station 2200. As shown, fluid transfer probe washing station 2200 includes two wash reservoirs, namely, wash reservoir 2202 and wash reservoir 2204. Wash reservoirs 2202 and 2204 typically contain different wash solvents (e.g., DMSO, ethanol, methanol, water, or the like). Wash reservoir 2202 fluidly communicates with overflow reservoir 2206, which fluidly communicates with waste reservoir 2208 via a fluid conduit. As shown, fluid sensor 2210 is disposed in sensory communication with the fluid conduit between wash reservoir 2202 and overflow reservoir 2206 to sense fluid disposed proximal to (e.g., leakage from, etc.) the fluid conduit. Fluid sensor 2212 is disposed in sensory communication with waste reservoir 2208 to sense fluid disposed proximal to and/or the fluid level in waste reservoir 2208. Fluid sensors utilized in fluid transfer probe washing station 2200 are optionally wet or dry sink fluid presence sensors. In addition, the fluid sensors of fluid transfer probe washing station 2200 are typically operably connected to one or more controllers, which receive data from the fluid sensors to monitor the presence of fluid in and/or proximal to fluid transfer probe washing station 2200. Controllers are described in greater detail below. Wash reservoir 2202 and waste reservoir 2208 also fluidly communicate with one another via valve 2214 (e.g., a three-way pinch valve or the like), fluid sensor 2216, and pump 2218 (e.g., a peristaltic pump, etc.). Pump 2218 effects fluid flow between wash reservoir 2202 and waste reservoir 2208.

As additionally shown in FIG. 22, wash reservoir 2204 fluidly communicates with overflow reservoir 2220, which fluidly communicates with waste reservoir 2222 via a fluid conduit. As also shown, fluid sensor 2224 is disposed in sensory communication with the fluid conduit between wash reservoir 2204 and overflow reservoir 2222 to sense fluid disposed proximal to (e.g., leakage from, etc.) the fluid conduit. Fluid sensor 2226 is disposed in sensory communication with waste reservoir 2208 to sense fluid disposed proximal to and/or the fluid level in waste reservoir 2222. Wash reservoir 2204 and waste reservoir 2222 also fluidly communicate with one another via valve 2228 (e.g., a three-way pinch valve or the like), fluid sensor 2230, and pump 2232 (e.g., a peristaltic pump, etc.). Pump 2232 effects fluid flow between wash reservoir 2204 and waste reservoir 2222. Valves 2214 and 2228, fluid sensors 2216 and 2230, and pumps 2218 and 2232 are typically housed in electronics box 2234. In addition, one or more controllers (e.g., pump and valve controllers, etc.) and a power supply are also optionally housed in electronics box 2234.

iii. Electromagnetic Radiation Sources, Optical Systems, and Detectors

The assaying components of the systems of the invention are configured to detect and quantify absorbance, transmission, and/or emission of light, and/or changes in those properties in samples that are typically arrayed in the wells of a multi-well plate, or arrayed in dot blots supported on membranes, treated glass, or other support materials. The systems of the invention can also be used to detect and quantify these properties in irregularly distributed samples. In addition to other system components described herein, the assaying components of the systems of the invention also generally include illumination or electromagnetic radiation sources, optical systems, and detectors. Because the systems and methods of the invention are flexible and allow essentially any chemistry to be assayed, they can be used for all phases of assay development, including prototyping and mass screening.

In some embodiments, the assaying components of the systems of the invention are configured for area imaging, but can also be configured for other formats including as a scanning imager or as a nonimaging counting system. An area imaging system typically places an entire multi-well container or other specimen onto the detector plane at one time. Accordingly, there is typically no need to move photomultiplier tubes (PMTs), to scan a laser, or the like, because the detector images the entire container onto many small detector elements (e.g., charge-coupled devices (CCDs), etc.) in parallel. This parallel acquisition phase is typically followed by a serial process of reading out the entire image from the detector. Scanning imagers typically pass a laser or other light beam over the specimen, to excite fluorescence, reflectance, or the like in a point-by-point or line-by-line fashion. In certain cases, confocal-optics are used to minimize out of focus fluorescence. The image is constructed over time by accumulating the points or lines in series. Nonimaging counting systems typically use PMTs or light sensing diodes to detect alterations in the transmission or emission of light, e.g., within wells of a multi-well container. These systems then typically integrate the light output from each well into a single data point.

A wide variety of illumination or electromagnetic sources and optical systems can be adapted for use in the assaying components or other sub-systems of the systems of the present invention. Accordingly, no attempt is made herein to describe all of the possible variations that can be utilized in the systems of the invention and which will be apparent to one skilled in the art. Exemplary electromagnetic radiation sources that are optionally utilized in the systems of the invention include, e.g., lasers, laser diodes, electroluminescence devices, light-emitting diodes, incandescent lamps, arc lamps, flash lamps, fluorescent lamps, and the like. One preferred type of laser used in the assaying systems of the invention are argon-ion lasers. Exemplary optical systems that conduct electromagnetic radiation from electromagnetic radiation sources to sample containers and/or from sample containers to detectors typically include one or more lenses and/or mirrors to focus and/or direct the electromagnetic radiation as desired. Many optical systems also include fiber optic bundles, optical couplers, filters (e.g., filter wheels, etc.), and the like.

Suitable signal detectors that are optionally utilized in these systems detect, e.g., emission, luminescence, transmission, fluorescence, phosphorescence, absorbance, or the like. In preferred embodiments, the detector monitors a plurality of optical signals, which correspond in position to “real time” results. Example detectors or sensors include PMTs, CCDs, intensified CCDs, photodiodes, avalanche photodiodes, optical sensors, scanning detectors, or the like. Each of these as well as other types of sensors is optionally readily incorporated into the systems described herein. The detector optionally moves relative to multi-well plates or other assay components, or alternatively, multi-well plates or other assay components move relative to the detector. In certain embodiments, for example, detection components are coupled to translation components that move the detection components relative to multi-well plates positioned on container positioning devices of the systems described herein. Optionally, the systems of the present invention include multiple detectors. In these systems, such detectors are typically placed either in or adjacent to, e.g., a multi-well plate or other vessel, such that the detector is in sensory communication with the multi-well plate or other vessel (i.e., the detector is capable of detecting the property of the plate or vessel or portion thereof, the contents of a portion of the plate or vessel, or the like, for which that detector is intended). In certain embodiments, detectors are configured to detect electromagnetic radiation originating in the wells of a multi-well container.

The detector optionally includes or is operably linked to a computer, e.g., which has system software for converting detector signal information into assay result information or the like. For example, detectors optionally exist as separate units, or are integrated with controllers into a single instrument. Integration of these functions into a single unit facilitates connection of these instruments with the computer, by permitting the use of a few or even a single communication port for transmitting information between system components. Computers and controllers are described further below. Detection components that are optionally included in the systems of the invention are described further in, e.g., Skoog et al., Principles of Instrumental Analysis, 5^{th} Ed., Harcourt Brace College Publishers (1998) and Currell, Analytical Instrumentation: Performance Characteristics and Quality, John Wiley & Sons, Inc. (2000), which are incorporated by reference.

Additional details relating to electromagnetic radiation sources, optical systems, detectors, and other aspects of the present invention which can be utilized or adapted for use in the systems described herein are provided in, e.g., U.S. Pat. Nos. 6,316,774, entitled “OPTICAL SYSTEM FOR A SCANNING FLUOROMETER,” which issued Nov. 13, 2001 to Giebeler et al., 5,112,134, entitled “SINGLE SOURCE MULTI-SITE PHOTOMETRIC MEASUREMENT SYSTEM,” which issued May 12, 1992 to Chow et al., 5,766,875, entitled “METABOLIC MONITORING OF CELLS IN A MICROPLATE READER,” which issued Jun. 16, 1998 to Hafeman et al., 6,469,311, entitled “DETECTION DEVICE FOR LIGHT TRANSMITTED FROM A SENSED VOLUME,” which issued Oct. 22, 2002 to Modlin et al., 6,151,111, entitled “PHOTOMETRIC DEVICE,” which issued Nov. 21, 2000 to Wechsler et al., 6,498,690, entitled “DIGITAL IMAGING SYSTEM FOR ASSAYS IN WELL PLATES, GELS AND BLOTS,” which issued Dec. 24, 2002 to Ramm et al., and 6,313,471, entitled “SCANNING FLUOROMETER,” which issued Nov. 6, 2001 to Giebeler et al., which are each incorporated by reference.

E. Additional Material Handling Components

In addition to the material handling components described above, e.g., with respect to the dispensing devices of the automated cell culture passaging stations and the fluid transfer devices of the assaying components of the compound profiling systems of the invention, other material handling components are also optionally included. In certain embodiments, for example, cells are expanded to selected quantities and pooled performing for compound profiling assays. These pooled cells are then typically dispensed into assay plates or other containers using various dispensing devices. Once these assay plates have been prepared, test compounds or reagents are typically transferred into the assay plates, e.g., using the transfer devices of the assaying components described above. Exemplary material handling components that are optionally adapted to perform reagent or cell culture dispensing, container washing, and/or other material handling functions in the systems of the invention are described in, e.g., U.S. Provisional Patent Application No. 60/577,849, entitled “DISPENSING SYSTEMS, SOFTWARE, AND RELATED METHODS,” filed Jun. 7, 2004 by Chang et al., U.S. Provisional Patent Application No. 60/598,994, entitled “MULTI-WELL CONTAINER PROCESSING SYSTEMS, SYSTEM COMPONENTS, AND RELATED METHODS,” filed Aug. 4, 2004 by Micklash I I et al., International Publication No. WO 2004/091746, entitled “MATERIAL REMOVAL AND DISPENSING DEVICES, SYSTEMS, AND METHODS,” filed Apr. 7, 2004 by Micklash I I et al., U.S. patent application Ser. No. 11/003,026, entitled “MATERIAL CONVEYING SYSTEMS, COMPUTER PROGRAM PRODUCTS, AND METHODS,” filed Dec. 1, 2004 by Chang et al., U.S. Patent Publication No. US-2003/0175164, entitled “DEVICES, SYSTEMS, AND METHODS OF MANIFOLDING MATERIALS,” filed Sep. 18, 2003 by Micklash I I et al., U.S. Pat. No. 6,659,142, entitled “APPARATUS AND METHODS FOR PREPARING FLUID MIXTURES,” to Downs et al., and U.S. Pat. No. 6,827,113, entitled “MASSIVELY PARALLEL FLUID DISPENSING SYSTEMS AND METHODS,” filed Mar. 27, 2002 by Downs et al., which are each incorporated by reference. In addition, exemplary micro-well plate stations that are optionally adapted for use in the systems of the invention are also described in, e.g., Reidel et al. (2005) “Low Temperature Microplate Stations,” JALA 10:29-34, which is incorporated by reference.

Other automated devices that are optionally used in the systems of the invention are replating stations positioned at station locations in one or more work perimeters. These devices are typically used to replate or replicate a plurality of samples from one or more small sample plates into a single large sample plate. For example, compounds are optionally transferred or replated from 96 well to 384 well microtiter plates and/or from 384 to 1536-well plates. These stations generally use visual and readable controls to track the reformatting and allow the user to verify that the reformatting was successful. A Tecan Miniprep robotic station (Tecan US, Durham, N.C., USA), which comprises an automatic sample processor, is one example of a device that is suitable for replating operations.

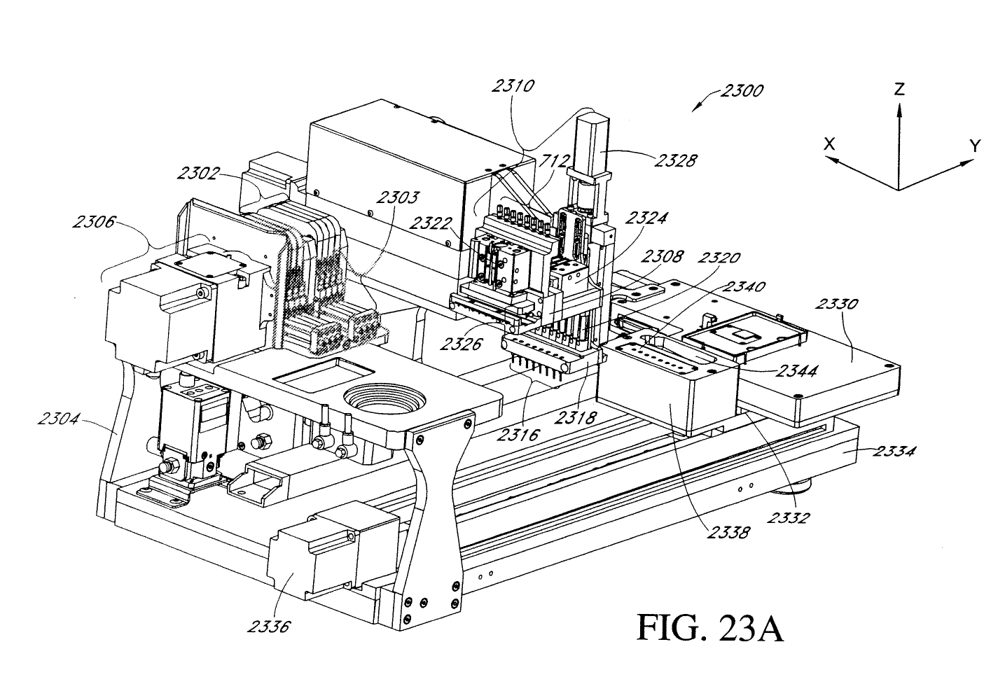

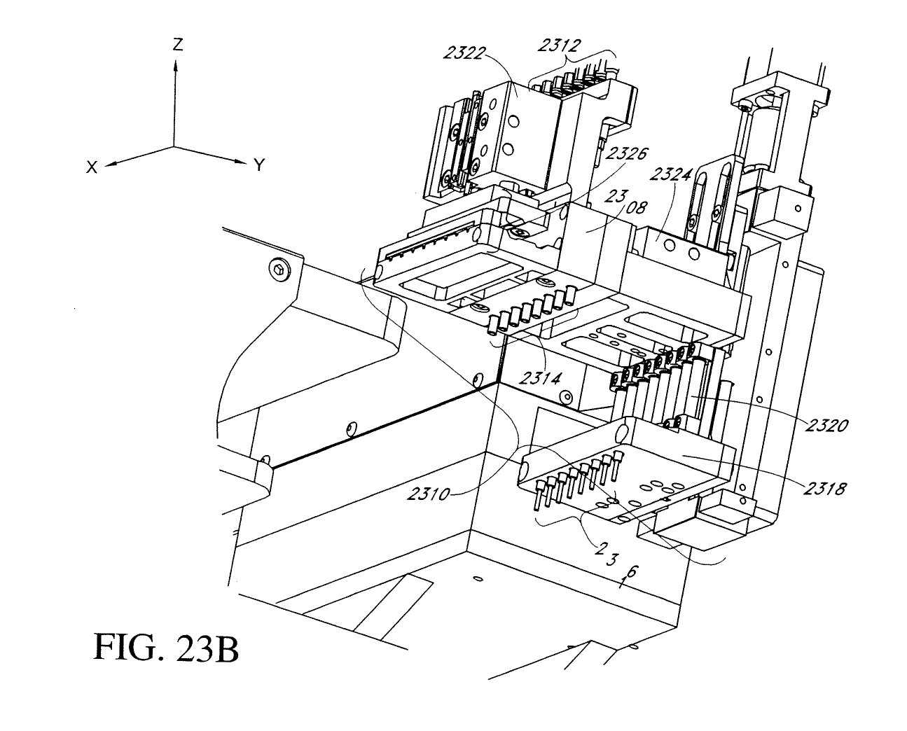

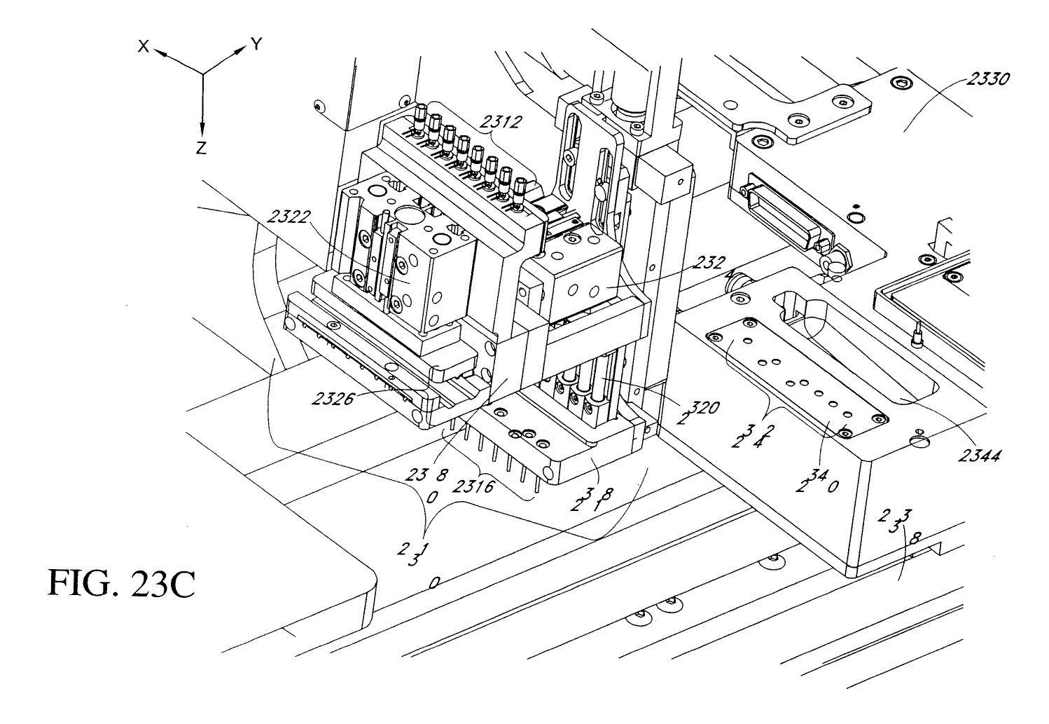

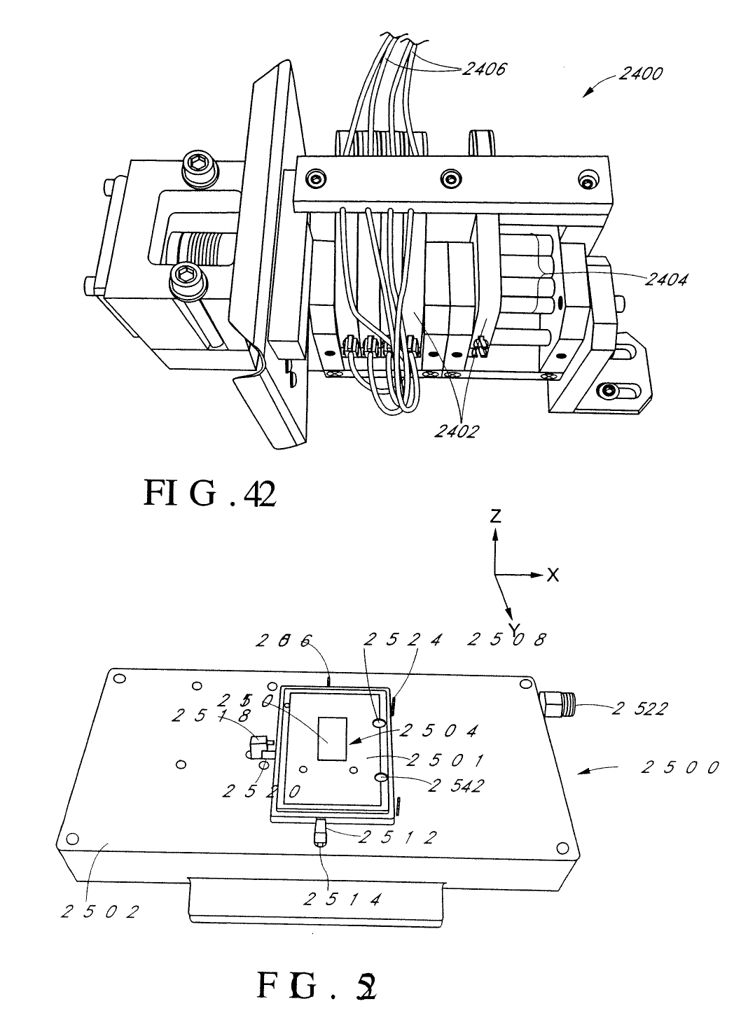

To further illustrate additional material handling components that are optionally included as components of the systems of the invention, FIGS. 23 A-C schematically depict dispensing station 2300 according to one embodiment. As shown, dispensing station 2300 includes peristaltic pump 2302 (e.g., a multi-channel low volume peristaltic pump) mounted on mounting component 2304 (shown as a rigid frame). Dispensing station 2300 also includes a feedback component that comprises drive motor 2306, which typically includes a position encoder and gear reduction, and which is operably connected to peristaltic pump 2302 to effect precisely controlled rotation of the rotatable roller support of peristaltic pump 2302. The feedback component also includes a control system for drive motor 2306 (not shown in FIG. 23) that is capable of position feedback control.

During operation, conduits (not shown in FIG. 23) are generally disposed between the compression surfaces and rollers of peristaltic pump 2302. In addition, one set of termini of the conduits fluidly communicate with the same or different material sources (not shown in FIG. 23), while the other set of termini are operably connected to and fluidly communicate with fluid junction block 2308 of dispensing component 2310. As also shown, dispensing station 2300 includes tube stretchers 2303, which are designed to give the user fine adjustment over the flow rate of each peristaltic channel. More specifically, tube stretchers 2303 mechanically increase the length of associated peristaltic tubing or conduits. As the length of a given tube is increased, the inner diameter of that tube decreases and the volume conveyed per pulse or rotational increment is also decreased. This gives the user a fine adjustment to the flow rate for each peristaltic channel. In some embodiments, further adjustments can be made by varying the spacing between peristaltic pump cartridges and rollers.

FIGS. 23 B and C schematically illustrate detailed bottom and top perspective views, respectively, of dispensing component 2310 from dispensing station 2300. Solenoid valves 2312 fluidly communicate with the same or different pressure sources (not within view) (e.g., a pressurized gas source, a pressurized second fluidic material source, a pump, etc.) and with fluid junction block 2308 via conduits (not shown in FIG. 23). Outlets 2314 of fluid junction block 2308 fluidly communicate with dispensing tips 2316 disposed in dispense head 2318 via conduits (not shown in FIG. 23), which conduits form conduit coils disposed around vertically mounted posts. As also shown, dispensing component 2310 also includes air tables 2322 and 2324. Air table 2322 effects operation of pinch valve 2326, whereas 2324 is operably connected to a gas valve (not within view) of fluid junction block 2308 to regulate the flow of gas into fluid junction block 2308 to introduce gaseous gaps to prevent fluid mixing.

In addition, dispensing component 2310 of dispensing station 2300 also includes Z-axis linear motion component 2328 (e.g., a compact, high speed, short travel Z-axis motion component or system), which is a positioning component that effects Z-axis translation of dispensing tips 2316 relative, e.g., multi-well plates, membranes, etc. disposed on object holder or container positioning device 2330. Object holder 2330 is operably connected to X/Y-axis linear motion components 2332 (shown as tables), which move object holder 2330 relative to dispensing tips 2316 along the X- and Y-axes. X/Y-axis linear motion components 2332 are also mounted on support element 2334, which forms part of mounting component 2304. One or more motors (e.g., solenoid motors, etc.) are generally operably connected to these dispensing stations to effect motion of object holders on X/Y-axis linear motion tables. For example, solenoid motor 2336 effects motion of object holder 2330 in dispensing station 2300. Although not within view in FIGS. 23 A-C, dispensing station 2300 also generally includes control drives, e.g., for X/Y-axis linear motion components 2332 and position feedback for drive motor 2306. As also shown, cleaning component 2338, which is used to clean dispensing tips 2316 is also included. In particular, cleaning component 2338 includes vacuum chamber 2340 having orifices 2342 that correspond to dispensing tips 2316 such that when dispensing tips 2316 are disposed proximal to orifices 2342 under a vacuum applied by vacuum chamber 2340, adherent material is removed at least from external surfaces of dispensing tips 2316. Cleaning component 2338 also includes fluid container 2344 disposed next to vacuum chamber 2340. In certain embodiments, fluid container 2344 contains a cleaning solvent into which dispensing tips 2316 can be lowered by Z-axis linear motion component 2328, e.g., prior to applying a vacuum to dispensing tips 2316 at vacuum chamber 2340. Optionally, fluid container 2344 is used as a waste collection component.

The dispensing stations of the systems of the invention also typically include controllers (also not shown in FIG. 23) that are configured to effect rotation of peristaltic pump roller supports in selected rotational increments, to effect application of pressure from pressure sources, to effect motion of linear motion components, and/or the like. These and other aspects of the systems invention are described further below.

i. Peristaltic Pumps

In certain embodiments, the dispensing stations of the systems of the invention generally include rotating peristaltic pumps with precisely regulated accelerations, velocities, and decelerations to effect accurate angular displacements. Essentially any rotary peristaltic pump can be used in the stations described herein. Peristaltic pumps typically use a turning mechanism to move fluids or other materials through a tube or other conduit that is compressed at a number of points in contact with, e.g., rollers, shoes, etc. of the pump such that the fluid is moved through the tube with each rotating motion. Peristaltic pumps generally include rotatable roller carriers or supports that support at least two rollers. Peristaltic pumps and related methods of pump control are also described in, e.g., U.S. patent application Ser. No. 11/003,026, entitled “MATERIAL CONVEYING SYSTEMS, COMPUTER PROGRAM PRODUCTS, AND METHODS,” filed Dec. 1, 2004 by Chang et al., which is incorporated by reference.

In some embodiments, for example, the peristaltic pump comprises a multi-channel peristaltic pump such that multiple quantities of material can be conveyed simultaneously. To illustrate, FIG. 24 schematically shows multi-channel peristaltic pump 2400 from a top perspective view. In the embodiment shown, multi-channel peristaltic pump 2400 comprises five channels 2402. Optionally, additional channels 2402 are added to multi-channel peristaltic pump 2400, or one or more of channels 2402 are removed from multi-channel peristaltic pump 2400. Typically, the number of channels is selected to correspond to the number of dispensing tips to be utilized in a dispensing station for a particular dispensing application. Rollers 2404 of the roller support of multi-channel peristaltic pump 2400 and conduits 2406 are also schematically shown in FIG. 24.

Although rotatable rollers (e.g., passively or actively rotatable) that rotate relative to roller supports are typically utilized in the systems of the invention, non-rotatable functionally equivalent components, such as fixed rollers or shoes are also optionally used. However, rotatable rollers generally produce less wear on material conduits (e.g., flexible tubing or the like) than non-rotatable equivalents for comparable amounts of usage.

Peristaltic pumps that can be adapted for use in the systems of the invention are available from a wide variety of commercial suppliers including, e.g., ABO Industries Inc. (San Diego, Calif., USA), Analox Instruments Ltd. (London, UK), ASF Thomas Industries GmbH (Puchheim, Germany), Barnant Co. (Barrington, Ill., USA), Cole-Parmer Instrument Company (Vernon Hills, Ill., USA), Fluid Metering Inc. (Syosset, N.Y., USA), Gorman-Rupp Industries (Bellville, Ohio, USA), I & J Fisnar Inc. (Fair Lawn, N.J., USA), Möler Feinmechanik GmbH & Co. (Fulda, Germany), PerkinElmer Instruments (Shelton, Conn., USA), Terra Universal Inc. (Anaheim, Calif., USA), and the like. Additional details relating to rotary pumps are described in, e.g., Karassik et al. (Eds.), Pump Handbook, The McGraw-Hill Companies (2000) and Nelik, Centrifugal and Rotary Pumps: Fundamentals with Applications, CRC Press (1999), which are both incorporated by reference.

ii. Motion Control

The motion control systems used in certain dispensing stations used in systems of the invention typically include matched components such as controllers, motor drives, motors, encoders and resolvers, user interfaces and software. Peristaltic pump drive motors generally include at least one position encoder and at least one gear reduction component. Exemplary motors utilized in these stations typically include, e.g., servo motors, stepper motors, or the like. In some embodiments, feedback components of these dispensing stations include at least one drive mechanism that is operably connected to the motor. The drive mechanism typically includes at least one control component that effects position feedback control of the motor.

As referred to above, the movement of peristaltic pump roller supports is typically effected by a motor operably connected to the pump. Exemplary motors that are optionally utilized in the systems of the invention include, e.g., DC servomotors (e.g., brushless or gear motor types), AC servomotors (e.g., induction or gearmotor types), stepper motors, linear motors, or the like. Servomotors typically have an output shaft that can be positioned by sending a coded signal to the motor. As the input to the motor changes, the angular position of the output shaft changes as well. Stepper motors generally use a magnetic field to move a rotor. Stepping can typically be performed in full step, half step, or other fractional step increments. Voltage is applied to poles around the rotor. The voltage changes the polarity of each pole, and the resulting magnetic interaction between the poles and the rotor causes the rotor to move.

The dispensing stations of the systems of the invention also generally include motor drives (e.g., AC motor drives, DC motor drives, servo drives, stepper drives, etc.), which act as interfaces between controllers and motors. In certain embodiments, motor drives include integrated motion control features. For example, servo drives typically provide electrical drive output to servo motors in closed-loop motion control systems, where position feedback and corrective signals optimize position and speed accuracy. Servo drives with integrated motion control circuitry and/or software that accept feedback, provide compensation and corrective signals, and optimizes position, velocity, and acceleration.

Suitable motors and motor drives are generally available from many different commercial suppliers including, e.g., Yaskawa Electric America, Inc. (Waukegan, Ill., USA), AMK Drives & Controls, Inc. (Richmond, Va., USA), Enprotech Automation Services (Ann Arbor, Mich., USA), Aerotech, Inc. (Pittsburgh, Pa., USA), Quicksilver Controls, Inc. (Covina, Calif., USA), NC Servo Technology Corp. (Westland, Mich., USA), HD Systems Inc. (Hauppauge, N.Y., USA), ISL Products International, Ltd. (Syosset, N.Y., USA), and the like. Additional detail relating to motors and motor drives are described in, e.g., Polka, Motors and Drives, ISA (2002) and Hendershot et al., Design of Brushless Permanent-Magnet Motors, Magna Physics Publishing (1994), which are both incorporated by reference.

iii. Pressure Sources

The dispensing stations of the systems of the invention typically include pressure sources in addition to the peristaltic pumps that convey fluidic materials into the stations in preparation for dispensing. These additional pressure sources are generally configured to apply pressure in station conduits such that selected aliquots of the fluidic materials (e.g., cell culture media, etc.) that have been conveyed into the station by the peristaltic pumps are forced or otherwise dispensed from the conduits. Essentially any pressure source can be adapted to effect fluidic material dispensing in this manner. To illustrate, pressure sources comprise pressurized gas sources that fluidly communicate with conduits from which fluidic materials are dispensed are used in certain embodiments. A wide variety of pressurized gas can be utilized. In some embodiments, for example, air compressors are used to provide air pressure to force the selected aliquots from system conduits. Other gases, such as nitrogen, helium, argon, or the like are also optionally used to effect fluidic material conveyance. In some embodiments, these pressurized gas sources fluidly communicate with conduits from which fluidic materials are dispensed via one or more fluidic material sources, such as a system fluid source (e.g., a buffer or other solvent). In these embodiments, the pressurized gas typically forces fluidic material from these pressurized fluidic material sources into these conduits to effect the dispensing of selected fluidic material aliquots from the conduits. Various pumps, such as syringe pumps, other peristaltic pumps, etc. can also be configured to function as these pressure sources in the dispensing systems described herein.

The pressure applied by these pressure sources to effect dispensing of selected fluidic material aliquots can be regulated using a wide variety of techniques. In certain embodiments, for example, valves are positioned between pressure sources and the openings of conduits from which fluidic materials are dispensed. In some of these embodiments, solenoid valves, such as microsolenoid valves are utilized. Suitable valves are commercially available from various suppliers including, e.g., The Lee Company USA (Westbrook, Conn., USA). In these embodiments, valves are typically operably connected to controllers, which effect operation of the valves. Controllers are described in greater detail below.

iv. Positioning and Mounting Components

In some embodiments, the dispensing stations of the systems of the invention include positioning components. Positioning components are generally structured to moveably position conduits and/or fluidic material sites relative to one another. Positioning components typically include at least one object holder or container positioning device that is structured to support the fluidic material site (e.g., a multi-well plate, a substrate, etc.). Typically, positioning components are operably connected to system controllers, which are configured to simultaneously effect fluidic material dispensing from conduits and moveably position the conduits and/or fluidic material sites relative to one another such that fluidic material volumes are conveyed to the fluidic material sites synchronous with the relative movement of the conduits and/or the fluidic material sites, e.g., to effect high throughput “on-the-fly” fluidic material dispensing.

For positioning along two different axes, the object holders of the dispensing systems of the invention generally have one or more alignment members positioned to receive, e.g., each of the two axes of a multi-well container. For example, FIG. 25 shows a top perspective view of object holder 2500 that can be used in the dispensing systems described herein. Another embodiment of an object holder (i.e., object holder 2330) is schematically depicted in FIG. 23A, which is described further above. As shown in FIG. 25, container station 2501 is disposed on support structure 2502 of object holder 2500. Support structure 2502 supports vacuum plate 2504. Protrusions 2506 and 2508 function as alignment members. The illustrated embodiment of the container station 2501 has two x-axis protrusions 2508 and one y-axis protrusion 2506 extending from support structure 2502. Accordingly, x-axis protrusions 2508 and y-axis protrusion 2506 are fixedly positioned relative to the vacuum plate 2504, which, in this embodiment, acts to hold a multi-well container in position once it has been positioned. X-axis locating protrusions 2508 are constructed to cooperate with an x-axis surface of a multi-well container (e.g., a y-axis wall of a microtiter plate), while y-axis protrusion 2506 is constructed to cooperate with an y-axis surface of the container (e.g., a y-axis wall of a microtiter plate).

The alignment members can be, for example, locating pins, tabs, ridges, recesses, or a wall surface, and the like. In some embodiments, an alignment member includes a curved surface that contacts a properly positioned multi-well container. The use of a curved surface minimizes the effect of, for example, roughness of the container surface that contacts the alignment member. The use of two alignment members along one axis and one alignment member along the second axis, as shown in FIG. 25, is another approach to minimize the effect of surface irregularities on the proper positioning of the container. The multi-well container contacts three points along the surface of the container, so proper alignment is not dependent upon the entire container surface being regular.

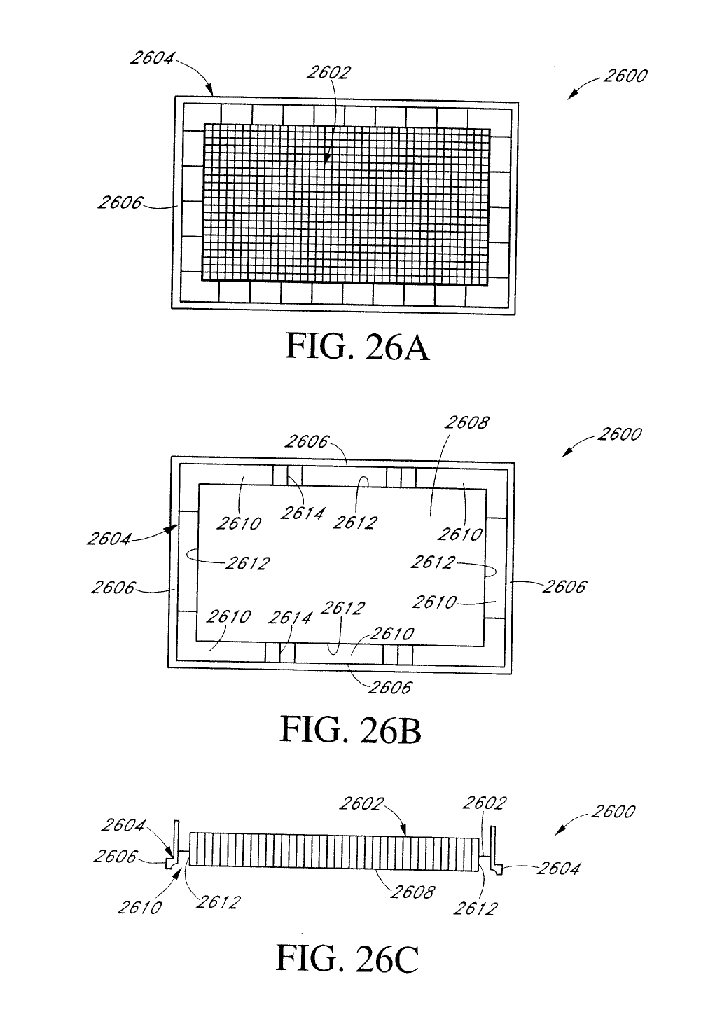

Certain aspects of the invention apply specifically to the positioning of microtiter plates, e.g., when used as assaying plates, compound plates, or the like. To illustrate, microtiter plate 2600 is shown in FIGS. 26A-C. As shown, microtiter plate 2600 comprises well area 2602, which has many individual sample wells for holding samples and reagents. Microtiter plates are available in a wide variety of sample well configurations, including commonly available plates with 6, 12, 24, 48, 96, 192, 384, 768, 1536, 9600, or more wells. It will be appreciated that microtiter plates are available from a various manufacturers including, e.g., Greiner America Corp. (Lake Mary, Fla., USA), Nalge Nunc International (Rochester, N.Y., USA), and the like. Microtiter plate 2600 has outer wall 2604 having registration edge 2606 at its bottom. In addition, microtiter plate 2600 includes bottom surface 2608 below the well area on the plate's bottom side. Bottom surface 2608 is separated from outer wall 2604 by alignment member receiving area 2610. Alignment member receiving area 2610 is bounded by a surface of outer wall 2604 and by inner wall 2612 at the edge of bottom surface 2608. Although there may be some lateral supports 2614 in alignment member receiving area 2610, these areas are generally open between inner wall 2612 and an inner surface of the outer wall 2604.

In certain embodiments, to position a microtiter plate the alignment members of the container station are optionally arranged to cooperate with inner wall 2612 of the microtiter plate. Inner wall 2612 is advantageously used, as inner wall 2612 is typically more accurately formed and is more closely associated with the perimeter of the sample well area, as compared to an outer wall of plate 2600, such as wall 2604. Accordingly, aligning an inner wall (e.g., inner wall 2612) of a microtiter plate relative to alignment members is generally preferred to aligning with an outer wall, such as wall 2604. The increased positioning precision that is obtained by using an inner wall as the alignment surface makes possible the use of high-density microtiter plates, such as 1536-well plates. Further, by having the alignment members (e.g., alignment protrusions 2506 and 2508) cooperate with an inner wall 2612 of plate 2600, minimal structures are needed adjacent the outside of the plate. In such a manner, a robotic arm or other transport device is able to readily access plate 2600. Having the protrusions positioned adjacent inner wall 2612 thereby facilitates translocating plate 2600. However, it will be appreciated that the alignment members or protrusions can be placed in alternative positions and still facilitate the precise positioning of the plate.

Object holders generally include one or more movable members. The movable members function to move a container against one or more alignment members. For example, once a multi-well container is placed in the general location of the alignment members, the movable members (termed “pushers” herein) move the container so that an alignment surface of the container is in contact with one or more of the alignment members of the positioning device. The positioning device can have pushers for positioning of the container along one or more axes. For example, a positioning device will often have one or more pushers that position a container along an x-axis, and one or more additional pushers that position the container along a y-axis. The pushers can be moved by means known to those of skill in the art. For example, air cylinders, springs, pistons, elastic members, electromagnets or other magnets, gear drives, and the like, or combinations thereof, are suitable for moving the pushers so as to move containers into a desired position.

One embodiment of a container station of an object holder having pushers for positioning a microtiter plate along both the x-axis and the y-axis is shown in FIG. 25. When the microtiter plate is generally positioned adjacent the x- and y-axis protrusions, the bottom surface of the microtiter plate is directly above top surface 2510 of vacuum plate 2504. Y-axis pusher 2512, which extends through slot 2514 in support structure 2502, is used to apply pressure to a y-axis side wall of the microtiter plate. Sufficient force is applied to the plate to push the microtiter plate against y-axis protrusion 2506. When the microtiter plate is pushed against y-axis protrusion 2506, x-axis pusher 2518, which extends through slot 2520 of support structure 2502, is used to push an x-axis wall of the microtiter plate towards x-axis protrusions 2508. In this manner, the microtiter plate is accurately and precisely positioned relative both the x-axis and y-axis protrusions. It is sometimes advantageous, although not necessary, to have one or more of the pushers contact an inner wall of a microtiter plate rather than an outer wall. With this arrangement, the alignment members and pushers are underneath the microtiter plate. This leaves the area surrounding the exterior of the plate free of protrusions that could otherwise interfere with other devices that, for example, place the microtiter plate on the support.

As referred to above, the object holder embodiment shown in FIG. 25 includes vacuum plate 2504 that functions as a retaining device to hold a properly positioned container in a desired position. With both y-axis pusher 2512 and x-axis pusher 2518 applying sufficient force to precisely place the microtiter plate, a vacuum source (not shown) applies a vacuum through vacuum line 2522 into vacuum openings or holes 2524. Air source (not shown) applies air pressure through an air line (not shown) to effect movement of the pushers.

In certain embodiments, positioning components also include X/Y-axis linear motion tables operably connected to position feedback control drives that control movement of the X/Y-axis linear motion tables along X- and Y-axes. In certain embodiments, linear motion tables are configured to move only along a single axis, such as an X-axis or a Y-axis. Typically, object holders are mounted on, e.g., X/Y-axis linear motion tables. As an example, FIG. 23A schematically shows object holder 2330 mounted on X/Y-axis linear motion table 2332. Positioning components also generally include Z-axis linear motion components that include dispense heads (see, e.g., dispense head 2318 schematically shown in FIG. 23A) that supports portions of conduits and that move along the Z-axis. The Z-axis linear motion components generally include a solenoid motor or the like to effect movement of the dispense heads along the z-axis. In certain embodiments, Z-axis linear motion components also include material removal heads, e.g., mounted proximal to dispense heads. For example, certain material removal heads are configured to noninvasively remove materials from the wells of multi-well plates, e.g., to effect plate washing during certain applications. Material removal heads are typically structured to prevent cross-contamination among wells of multi-well plates as materials are removed from the plates. Additional details relating to material removal heads, systems and related methods, that are optionally adapted for use with the systems of the present invention are provided in, e.g., International Publication No. WO 2004/091746, entitled “MATERIAL REMOVAL AND DISPENSING DEVICES, SYSTEMS, AND METHODS,” filed Apr. 7, 2004 by Micklash I I et al., which is incorporated by reference.

Various other positioning components or portions thereof can be utilized in the systems of the invention. In certain embodiments, for example, detectable signals produced on, e.g., multi-well plates, substrate surfaces, etc. disposed on the object holders of the systems described herein are detected. In some of these embodiments, orifices are disposed through object holders to facilitate such detection. To further illustrate, object holders optionally comprise nests in which multi-well plates or other fluidic material sites can be positioned in some embodiments of the invention. Some of these devices are described above with respect to assaying components of the systems of the invention. These or other types of object holders that can be utilized in the work stations of the systems of the present invention are described in, e.g., International Publication No. WO 01/96880, entitled “AUTOMATED PRECISION OBJECT HOLDER,” filed Jun. 15, 2001 by Mainquist et al., U.S. patent application Ser. No. 10/911,238, entitled “MULTI-WELL CONTAINER POSITIONING DEVICES AND RELATED SYSTEMS AND METHODS,” filed Aug. 3, 2004 by Evans, U.S. patent application Ser. No. 10/911,388, entitled “NON-PRESSURE BASED FLUID TRANSFER IN ASSAY DETECTION SYSTEMS AND RELATED METHODS,” filed Aug. 3, 2004 by Evans et al., and U.S. Provisional Patent Application No. 60/645,502, entitled “MULTI-WELL CONTAINER POSITIONING DEVICES, SYSTEMS, COMPUTER PROGRAM PRODUCTS, AND METHODS,” filed Jan. 19, 2005 by Chang et al., which are each incorporated by reference.

In some embodiments, dispensing stations include mounting components that mount peristaltic pumps, pressure sources, controllers, positioning component, and/or other system components relative to one another. Mounting component are typically substantially rigid, e.g., fabricated from steel or other materials that can adequately support the other system components during operation of the system. An exemplary mounting component (i.e., mounting component 2304) is schematically depicted in FIG. 23A, which is described further above.

v. Cleaning Components

The dispensing stations of the systems of the invention optionally also include cleaning components that are structured to clean conduits (e.g., dispensing tips thereof), e.g., when positioning components move the conduits at least proximal to the cleaning components. As fluidic materials are dispensed, some fluid can wick up or otherwise adhere to the outer surface of dispensing tips. This generally leads to additional wicking if the adherent fluid is not removed from the tips, because as the surface finish of a tip becomes coated with fluid it tends to attracts more fluid, e.g., during subsequent dispensing steps. Moreover, this also typically leads to inaccurate quantities of material being dispensed, since wicked materials are not dispensed at the selected fluidic material sites and/or are dispensed at non-selected sites. This inaccuracy may be compounded when multiple quantities of material are simultaneously dispensed from multiple material conduits, because fluidic material wicking tends to occur at different rates at the material conduit tips. Accordingly, wicked fluidic material is generally cleaned from material conduit tips, e.g., between dispensing steps using a cleaning component in certain embodiments of the invention.

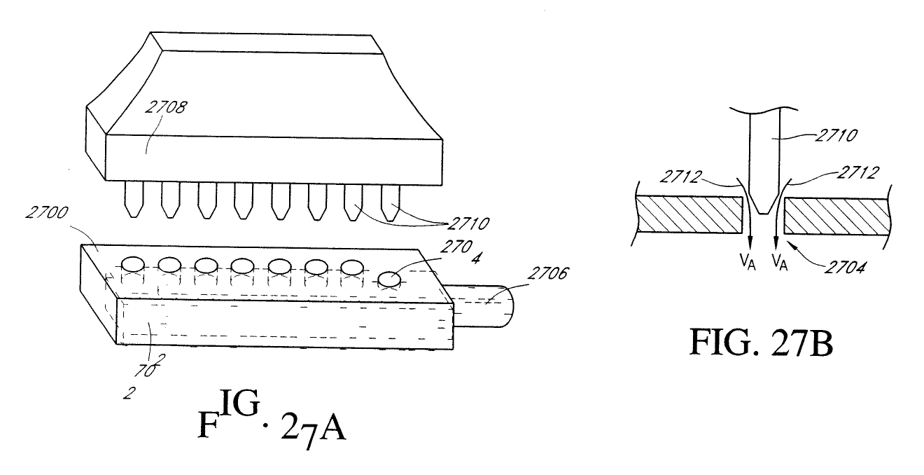

In some embodiments, for example, cleaning components include vacuum chambers that comprise at least one orifice into or proximal to which the positioning component moves the conduits such that an applied vacuum removes wicked or otherwise adherent material from external surfaces of the conduits or dispensing tips. Typically, outer cross-sectional dimensions of the conduits are smaller than cross-sectional dimensions of the orifices. To illustrate, FIG. 27A schematically shows a partially transparent perspective view of vacuum chamber 2702 of cleaning component 2700 according to one embodiment. As shown, multiple orifices 2704 are disposed in cleaning component 2700 and communicate with outlet 2706, which is typically operably connected to a vacuum source (not shown). Also shown is dispense head 2708 is disposed over cleaning component 2700. Orifices 2704 are structured to correspond to conduit tips 2710 of dispense head 2708 such that conduit tips 2710 can be lowered at least partially into orifices 2704 to effect removal of adherent materials from conduit tips 2710 under an applied vacuum. FIG. 27B schematically illustrates a detailed cross-sectional view of conduit tip 2710 disposed proximal to orifice 2704. Arrows 2712 represent the velocity of the air, VA, flowing through orifice 2704. As conduit tip 2710 is lowered into orifice 2704, the area of orifice 2704 is decreased such that VA increases in the gap that remains between vacuum chamber 2702 and conduit tip 2710 and pulls or otherwise removes adherent material from the outer surfaces of conduit tip 2710. Vacuum chambers are optionally disposed, e.g., on surfaces of object holders of the positioning components of the systems of the invention.

vi. Conduits

The conduits used in the systems of the invention include various embodiments. In some embodiments, for example, a terminus of a conduit used in a dispensing device includes a dispensing tip (e.g., a tapered tip, such as a nozzle or the like) that is fabricated integral with the conduit or is connected to the conduit, e.g., directly or via an insert. The size (e.g., internal cross-sectional dimension) of the conduit (e.g., pump tubing, etc.) and/or tip utilized is typically dependent, at least in part, on, e.g., the desired dispense volume, the viscosity of the fluidic material being conveyed, and the like. Although larger sizes are optionally utilized, cavities disposed through conduits and/or tips typically include, e.g., cross-sectional dimensions of between about 100 μm and about 100 mm, more typically between about 500 μm and about 50 mm, and still more typically between about 1 mm and about 10 mm. Optionally, cavities disposed through conduits or tips include at least two different cross-sectional dimensions.

Conduits, tips, and inserts are optionally fabricated from a wide variety of materials. Exemplary materials used to fabricated conduits, dispensing tips, and/or inserts include polypropylene, polystyrene, polysulfone, polyethylene, polymethylpentene, polydimethylsiloxane (PDMS), polycarbonate, polyvinylchloride (PVC), polymethylmethacrylate (PMMA), fluorinated ethylene propylene (FEP), polytetrafluoroethylene (PTFE) (TEFLON™), perfluoroalkoxy (PFA), autoprene, C-FLEX® (a styrene-ethylene-butylene (SEBS) modified block copolymer with silicone oil), NORPRENE® (a polypropylene-based material), PHARMED® (a polypropylene-based material), silicon, TYGON®, VITON® (includes a range of fluoropolymer elastomers), and the like. Dispensing tips and inserts are also optionally fabricated from other materials including glass and various metals (e.g., stainless steel, etc.). Materials for fabricating conduits, tips, and inserts are typically readily available from many different commercial suppliers including, e.g., Saint-Gobain Performance Plastics (Garden Grove, Calif., USA), DuPont Dow Elastomers L.L.C. (Wilmington, Del., USA), and the like.

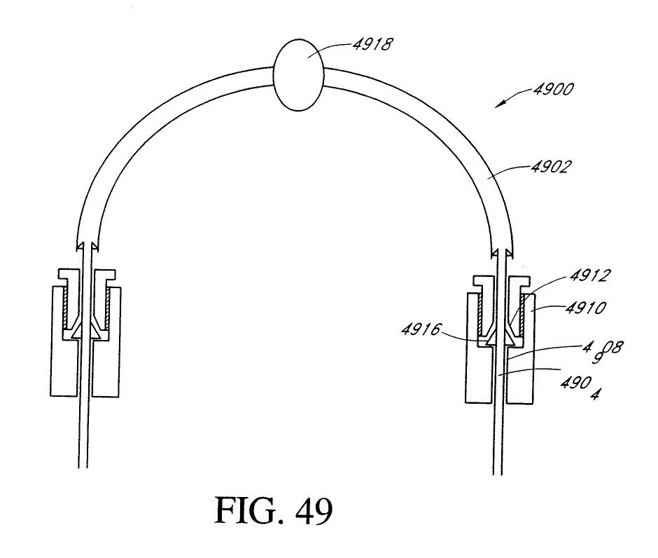

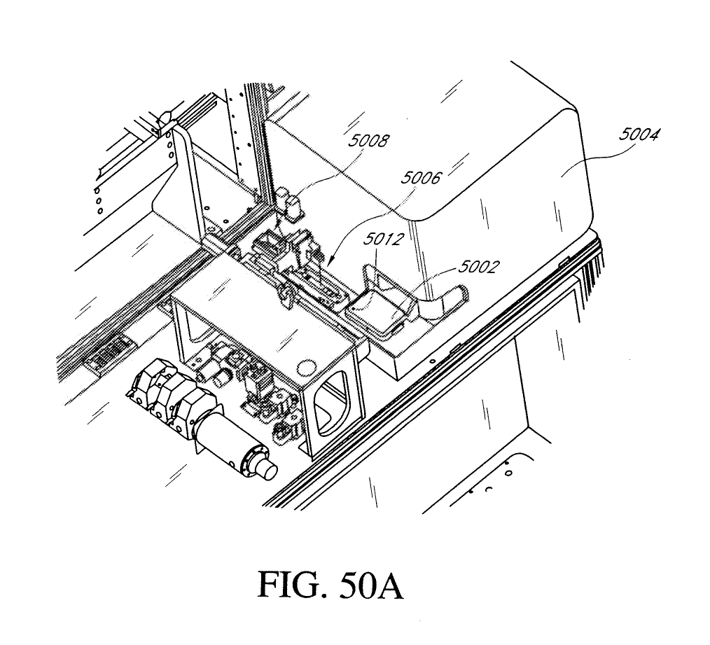

In certain embodiments, the conduits may comprise a resilient deformable material. As schematically illustrated in FIG. 49, an exemplary conduit 4900 comprises a length of tubing 4902, which is in fluid communication with tips 4904 at either end of the tubing. The tips 4904 comprise extensions which inhibit removal of the tips and which, preferably, form a seal which prevents fluid from leaking from the tubing. Preferably, the extensions comprise barb features 4906 which are tapered to facilitate introduction of the tips 4904 into the tube 4902, but taper outward to a diameter wide enough that a seal is formed with the interior walls of the tube 4902 and inhibit removal of the tips 4904 from the tube. Advantageously, the barb 4906 is tapered to avoid the formation of a crevice between the barb and the interior of the tubing 4906.

It can also be seen in the embodiment depicted in FIG. 49 that the tip 4904 extends through a hole 4908 in a housing 4910. In one embodiment, the hole 4908 is at least 5 times longer than the diameter of the hole. It can also be seen that the tip 4904 is held in place via a fastener. Preferably, the fastener is a threaded nut 4912, which has a tapered surface 4914 configured to mate with a tapered feature 4916 extending from the tip 4904, and comprises a through-hole permitting the tip to extend through the nut. The tapered surface 4914 serves to center the tip 4904 as the nut 4912 is tightened.

In the illustrated embodiment, as discussed above, the tubing 4902 comprises a peristaltic pump 4918 located between the tips 4904. In one embodiment, the pump 4918 is located closer to the suction side of the conduit 4900 in order to reduce the risk of the tubing 4902 collapsing due to minor plugs in the conduit 4900. By placing the pump 4918 directly between the tips 4904, the length of the tubing may be advantageously minimized.

In one embodiment, both tips 4904 may be cleaned in parallel, such as through the use of a cleaning component discussed above, or by routing a cleaning fluid through the conduit 4900. In addition, additional tips not in fluid communication with the tips 4904 may be cleaned at this time, as well.

F. Incubation, Refrigeration, and Container Storage Devices

The compound profiling systems of the invention optionally include various incubation, refrigeration, and storage stations that are within a work perimeter of, and accessible by, a given rotational robot or other robotic gripping device, e.g., at selected station locations. In certain embodiments, for example, incubation stations are used to culture cell populations, e.g., as part of an expansion or growth process prior to using the cells in a compound profiling process. In addition, as cell cultures are split using the cell culture passaging stations described above, sample aliquots are typically automatically removed from cell culture flasks at selected intervals and archived in freezer stations included in the systems of the invention. To further illustrate, compound and assay multi-well containers are also typically stored at least transiently in incubation, refrigeration, and other storage stations, e.g., prior to being utilized to perform a given assay in an assaying component of the system. Exemplary incubation and other storage devices that are optionally adapted for use in the systems of the invention are also described in, e.g., International Publication No. WO 03/008103, entitled “HIGH THROUGHPUT INCUBATION DEVICES,” filed Jul. 18, 2002 by Weselak et al., U.S. Patent Publication No. 2004/0236463, entitled “COMPOUND STORAGE SYSTEM,” filed Feb. 6, 2004 by Weselak et al., and U.S. Provisional Patent Application No. 60/598,929, entitled “OBJECT STORAGE DEVICES, SYSTEMS, AND RELATED METHODS,” filed Aug. 4, 2004 by Shaw et al., which are each incorporated by reference.

To further illustrate, incubation devices utilized in the systems of the invention typically include a housing with a plurality of doors disposed in, e.g., an access panel located on a side of the device. Typically, a robotic gripping device located outside the incubation device is used to open individual doors located in the access panel as it loads or unloads containers (e.g., multi-well containers, cell culture flasks, etc) into or out of the incubation device. This generally reduces the air exchange between the external environment and the internal environment of the incubation device along with limiting the moving parts within the interior of the incubation device. As a result, the incubation devices used in the systems of the invention provide a controlled environment for maintaining parameters, such as humidity, temperature, gas conditions (e.g., CO₂, N₂, or other gas levels).

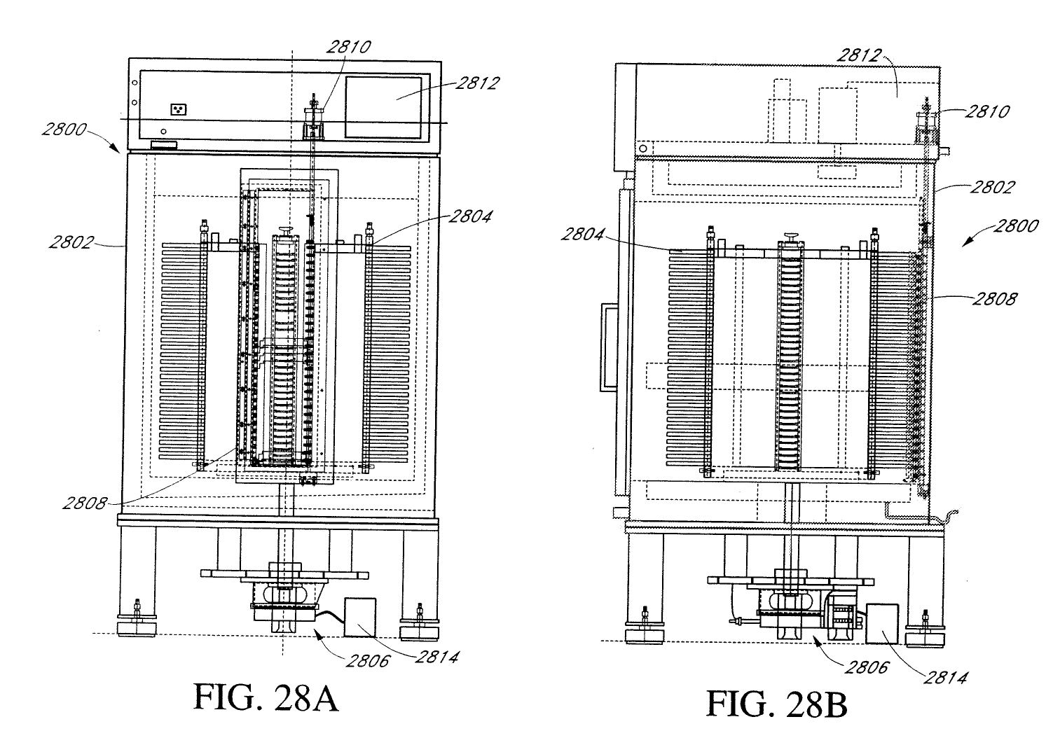

One embodiment of an incubation device is illustrated schematically in FIG. 28. In particular, FIG. 28A schematically depicts a front cutaway view of incubation device 2800. As shown, incubation device 2800 includes housing 2802 having carrousel with vertical columns of shelves 2804 disposed in housing 2802. Rotational mechanism 2806 (shown as an external motor) is operably connected to carrousel 2804 to rotate selected vertical columns of carrousel 2804 into alignment with vertical column of doors 2808. In certain embodiments, rotational mechanisms are configured to rotate the rotatable carrousels in one or more selectable modes. To illustrate, one exemplary selectable mode includes an oscillation (e.g., a side-to-side motion, etc.) of rotatable carrousels as the rotatable carrousels are rotated, e.g., to agitate containers or other objects disposed on the shelves of the carrousels. Typically, controller 2814 controls rotation of carrousel 2804 via rotational mechanism 2806, e.g., in these selectable modes. Incubation device 2800 also includes controller 2812, which controls one or more internal housing conditions. FIG. 28A also schematically illustrates door hold-open mechanism 2810 that includes a member (e.g., a rod, a column, a pole, a slat, a bar and the like) having a plurality of prongs (or a series of pins or other stops) for holding accessed doors of vertical column of doors 2808 open. FIG. 28B schematically depicts incubation device 2800 from a side cutaway view.

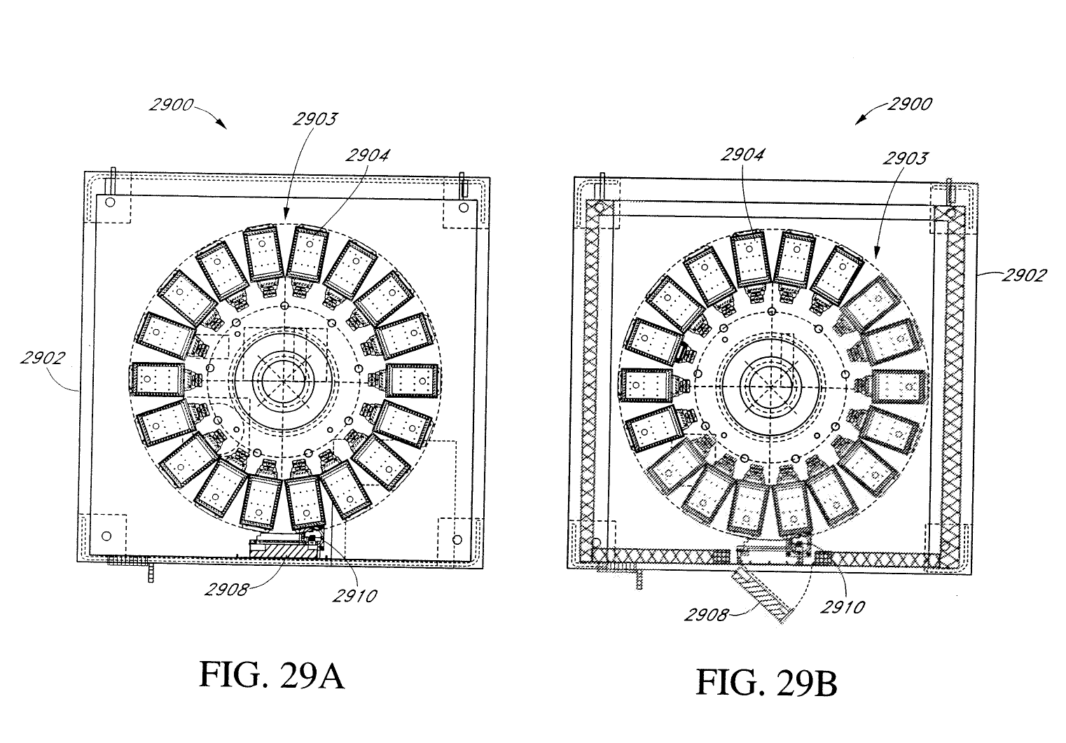

As referred to above, a rotating vertical carrousel with multiple columns (commonly referred to as “hotels”) and multiple shelves is typically located inside the incubation devices. To further illustrate, FIG. 29A schematically depicts a top cutaway view of incubation device 2900, while FIG. 29B schematically depicts a bottom cutaway view of incubation device 2900 according to one embodiment. Incubation device 2900 includes carrousel 2903 with a plurality of shelves 2904 disposed in housing 2902. A rotational mechanism (not shown) is operably connected to carrousel 2903 to rotate selected vertical columns of carrousel 2903 (e.g., about a Z-axis) into alignment with vertical column of doors 2908. Incubation device 2900 also includes door hold-open mechanism 2910 that includes a member (e.g., a rod, a column, a pole, a slat, a bar and the like) having a plurality of stops (shown as prongs) for holding accessed doors of vertical column of doors 2908 open. Vertical column of doors 2908 is hinged to housing 2902, which provides the ability to open or close vertical column of doors 2908. FIG. 29A schematically depicts vertical column of doors 2908 in a closed position, while FIG. 29B schematically depicts vertical column of doors 2908 in an open position.

As referred to above, the incubation devices of system of the invention optionally include access panels (e.g., vertical access panels, horizontal access panels, etc.), which are typically located on the sides of the devices. In some embodiments, access panels are attached to device housings via hinges. An open access panel provides access to a plurality of shelves in a carrousel and the interior compartment of the particular incubation device. Optionally, the access panel includes a gasket to further seal the interior environment of the given incubation device from the exterior environment and a lock, latch, and/or other mechanism to maintain the access panel in a closed position when desired.

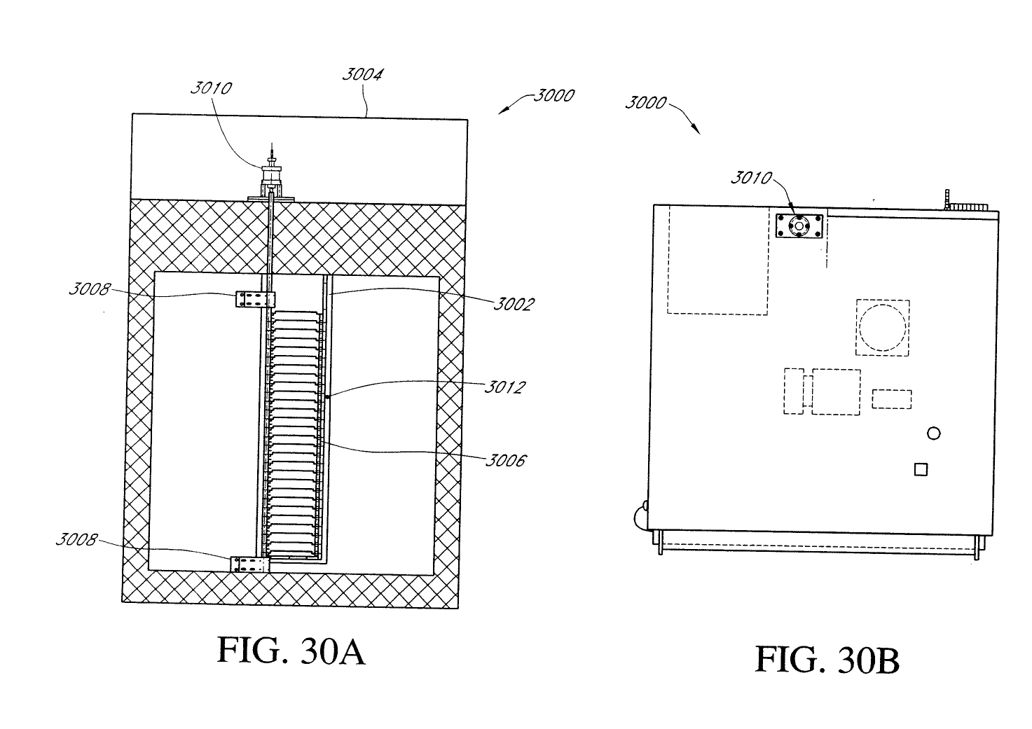

FIG. 30A schematically depicts a front view of incubation device 3000 according to one embodiment. As shown, access panel 3002 is disposed in a surface of device housing 3004. Access panel 3002 includes vertical column of doors 3006 and is attached to device housing 3004 by hinges 3008. A portion of door hold-open mechanism 3010 is also illustrated. FIG. 30B schematically depicts a top view of incubation device 3000.

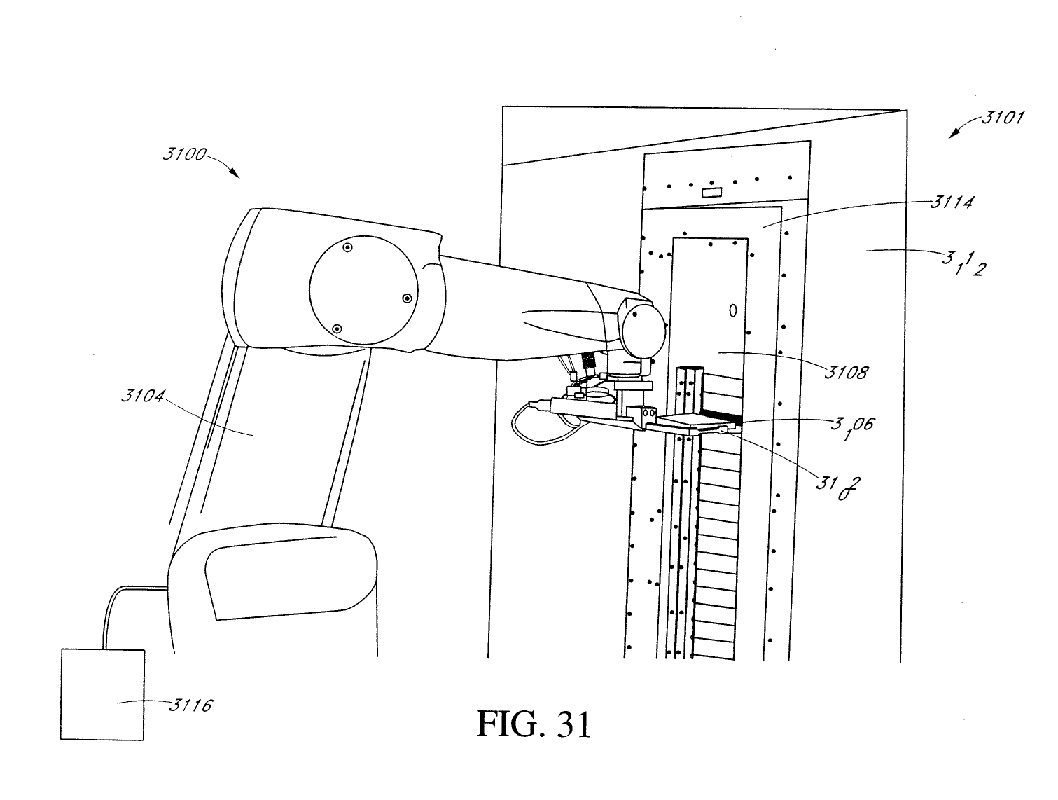

Individual actuators are typically not needed to open doors because a robotic gripping device typically provides mechanical actuation to open selected doors. Thus, incubation devices need not have any internal mechanism for opening the doors in, e.g., a given vertical column or horizontal row of doors. Since only relatively small doors are open at a time, air exchange between the interior of an incubation device and the outside atmosphere is reduced. FIG. 31 depicts robotic gripping device 3100 (e.g., a rotational robot) located outside incubation device 3101 opening door 3106 on vertical access panel 3114. Robotic gripping device 3100 loads and unloads containers into and out of incubation device 3101. More specifically, FIG. 31 schematically depicts gripper mechanism 3102 of robotic gripping device 3104 interfacing with door 3106 in vertical column of doors 3108 of housing 3112 in this exemplary embodiment. Robotic gripping device 3100 also includes logical device 3116 for controlling movement of robotic armature 3104. Robotic gripping devices are also described above.

The systems of the invention optionally include other storage devices, including certain modular object storage devices. These devices can be used, e.g., to store and manage large numbers of objects, such as compound libraries stored in multi-well containers. Robotic gripping devices are generally configured to translocate multi-well plates, substrates, cell culture flasks, or the like to and/or from object storage module shelves, and/or object storage modules to and/or from object storage module receiving areas of support elements of these modular object storage devices. As described above, system components such as these are optionally housed within enclosures or chambers, e.g., to prevent the contamination of objects stored on the shelves of modular object storage devices.

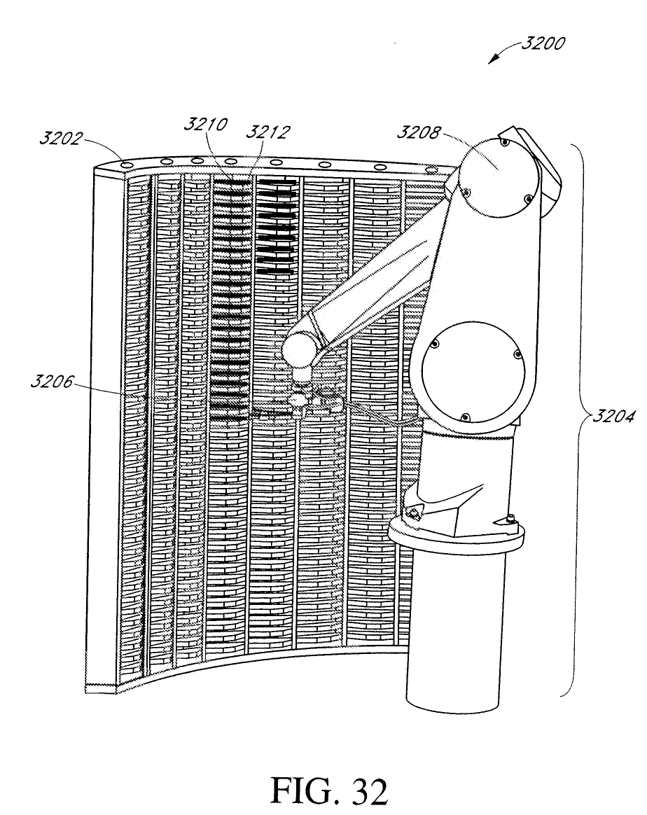

To illustrate, FIG. 32 schematically illustrates container storage station 3200, which includes modular object storage device 3202 and robotic gripping device 3204 from a perspective view. As shown, robotic gripping device 3204 includes gripper mechanism 3206 operably connected to robotic armature or boom 3208, which positions gripper mechanism 3206 relative to multi-well plates 3210 such that multi-well plates 3210 can be grasped by gripper mechanism 3206 and translocated to and/or from shelves 3212 of modular object storage device 3202 by boom 3208. Typically, robotic gripping device 3204 translocates multi-well plates 3210 between modular object storage device 3202 and another system component, such as a dispensing station, an assaying component, or other work station, e.g., for processing or analysis.

G. Lid Processing Devices

To reduce contamination and evaporative effects, it is sometimes desirable to provide sample containers with lids. A lid that sufficiently seals a given container, such as a multi-well container not only reduces evaporation and contamination, but also generally allows gases to diffuse into sample wells more consistently and reliably. Lids typically have a gripping structure, such as a gripping edge, that a robotic gripping device engages when adding or removing the lids from the containers. For example, U.S. Pat. No. 6,534,014, entitled “SPECIMEN PLATE LID AND METHOD OF USING,” filed May 11, 2000 by Mainquist et al., which is incorporated by reference, discloses specimen plate lids for robotic use that are optionally utilized to seal containers in the systems described herein. Further, lid processing devices or stations are also optionally included as components of the systems described herein, e.g., for adding and removing lids to and from containers.

H. Additional Detection Components

The systems of the invention also generally include detectors or detection components that are structured to detect detectable signals produced, e.g., in the wells of multi-well containers, in cell culture flasks, in samples aliquots taken from cell culture flasks, or the like. As described above, for example, detectors are typically included in the assaying components of the systems of the invention. Optionally, other detection components are included in these systems in addition to or in lieu of the assaying components described above.

To illustrate, suitable signal detectors that are optionally utilized in the systems of the invention detect, e.g., fluorescence, phosphorescence, radioactivity, mass, concentration (e.g., reagent concentrations, cellular concentrations or cell counts, etc.), pH, charge, absorbance, refractive index, luminescence, temperature, magnetism, or the like. In one exemplary embodiment, an ACQUEST™ workstation (Molecular Devices Corp., Sunnyvale, Calif., USA) is included as a system component. These workstations typically include multi-mode readers and modified nests for robotic access. In some embodiments, the systems of the invention also include FACS arrays or other cell counting components. Examples of these components that are optionally adapted for use in the systems described herein include the BD FACSArray™ bioanalyzer system (BD Biosciences, San Jose, Calif., USA), the MetaMorph® Imaging System (Universal Imaging Corporation™ a subsidiary of Molecular Devices, Downingtown, Pa., USA), or the like.

Detectors optionally monitor one or a plurality of signals from upstream and/or downstream of the performance of, e.g., a given assay or processing step. For example, the detector optionally monitors a plurality of optical signals, which correspond in position to “real time” results. Example detectors or sensors include photomultiplier tubes, CCD arrays, optical sensors, temperature sensors, pressure sensors, pH sensors, conductivity sensors, scanning detectors, or the like. Each of these as well as other types of sensors is optionally readily incorporated into the systems described herein. Detectors are optionally configured to move relative to multi-well containers, cell culture flasks, or other components, or alternatively, multi-well containers, cell culture flasks, or other components are configured to move relative to the detector. In certain embodiments, for example, detection components are coupled to translation components that move the detection components relative to multi-well containers, cell culture flasks, or other containers positioned on object holders or container positioning devices described herein. Optionally, the systems of the present invention include multiple detectors. In these systems, such detectors are typically placed either in or adjacent to, e.g., a multi-well container or other vessel, such that the detector is within sensory communication with the multi-well container or other vessel (i.e., the detector is capable of detecting the property of the plate or vessel or portion thereof, the contents of a portion of the plate or vessel, or the like, for which that detector is intended).

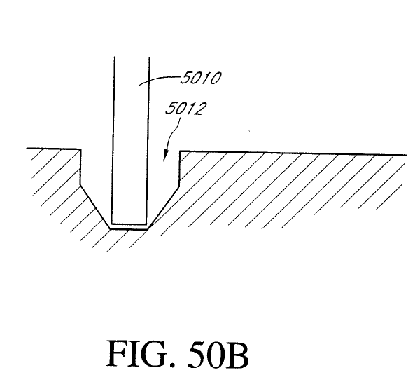

In one embodiment, described with respect to FIGS. 50A and 50B, the detectors may be used in conjunction with a reusable well plate. In the illustrated embodiment, the reusable well plate 5002 is a single-well plate, which has the same shape and footprint as a standard 96-well plate, and is thus compatible with a detector which is configured to receive a 96-well plate. In other embodiments, the well plate may have more than one well. In the illustrated embodiment, the detector is a FACS array 5004. In order to facilitate cleaning, the reusable well plate may advantageously be formed from stainless steel.

A sample may be added to the well via sample tip 5006. In one embodiment, the sample is drawn directly from a flask, without transferring the sample to an intermediate container. Advantageously, this minimizes the amount of replacement and cleaning of components. The sample tip may be rinsed with cleaning reagents both prior to and after addition of the sample to the well. In further embodiments, a reagent addition tip (not shown) may be utilized to add an additional component, such as a stain, to the well.

After the sample has been added to the well, the detector, which in this embodiment is the FACS array 5004, is commanded to read the sample and output the cell density of the sample. Advantageously, the sample tip 5006 may be cleaned in parallel with this process, so as to maximize throughput. After the detection process has been completed, the well plate 5002 is ejected for cleaning. In the illustrated embodiment, the sample may be removed from the container via an aspirating tip 5010 (not shown). Cleaning reagent may then be added to the well via one or more cleaning reagent tips 5008, and aspirated via the aspirating tip.

FIG. 50B is a schematic cross-section of the well 5012 of the reusable well plate 5002. As can be seen, the sides of the well 5012 taper inward, such that the base 5014 of the well is substantially equal in size and shape to the cross-section of the end of the aspirating tip 5010. This advantageously permits the removal via the aspirating tip 5010 of as much of the fluid in the well 5012 as possible.

Detectors optionally include or are operably linked to a computer, e.g., which has system software for converting detector signal information into assay result information or the like. For example, detectors optionally exist as separate units, or are integrated with controllers into a single instrument. Integration of these functions into a single unit facilitates connection of these instruments with the computer, by permitting the use of few or a single communication port(s) for transmitting information between system components. Computers and controllers are described further below. Detection components that are optionally included in the systems of the invention are described further in, e.g., Skoog et al., Principles of Instrumental Analysis, 5^{th} Ed., Harcourt Brace College Publishers (1998) and Currell, Analytical Instrumentation: Performance Characteristics and Quality, John Wiley & Sons, Inc. (2000), which are both incorporated by reference.

I. Fermentors

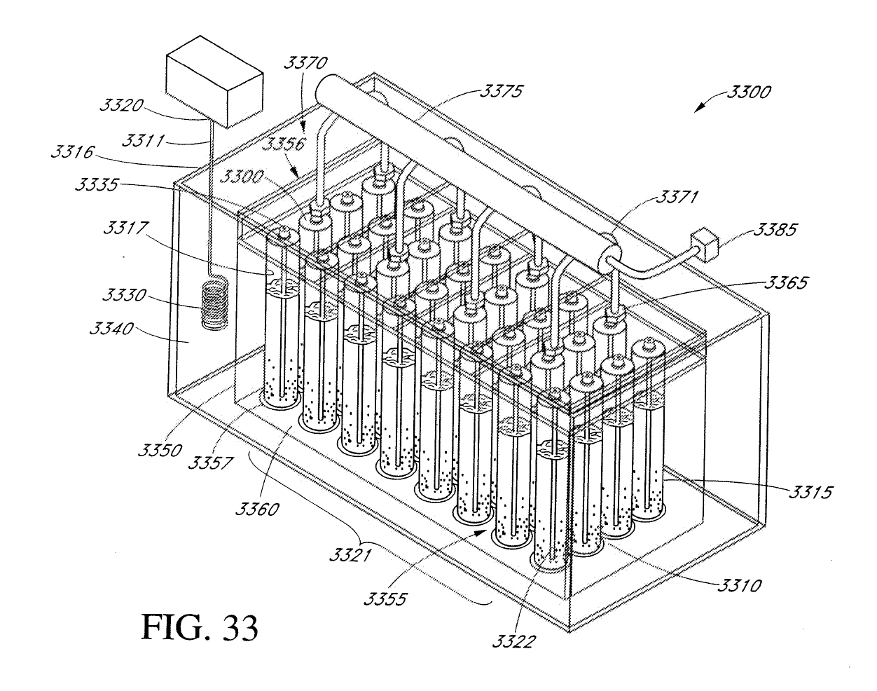

Fermentation stations are optionally included as components of the systems described herein. In certain embodiments, for example, fermentors are used to grow cell populations as part of various cell culturing processes. An exemplary fermentor is provided in FIG. 33. Fermentor 3300 generally comprises sample holder arrangement 3355, cannula assembly 3380 and gas distribution arrangement 3370. The illustrated fermentor 3300 is configured to separately and simultaneously ferment multiple batch samples in sample vessels that are compatible with direct pre- and post-fermentation processing.

Sample holder arrangement 3355 includes gripping surfaces 3317, individual sample vessels 3315, which typically form an array of sample vessels, such as array 3310, a transportable container frame 3350, and an array of placement wells 3360 corresponding to array 3310. Gripping surfaces 3317 are optionally located on each individual sample vessel 3315, which collectively form sample vessel array 3310. Typically, gripping surface 3317 resides on the bottom of each sample vessel, but gripping surface 3317 is optionally located on any surface of the sample vessel that enables sample vessel 3315 to be transferred to or from container frame 3350 or another processing station.

The bottom of each individual sample well 3315 is positioned within a placement well, e.g., placement well 3357. The array of placement wells 3360 typically mirrors the configuration of array 3310 and is embedded in transportable container frame 3350.

By using transportable container frame 3350, the entire array of sample vessels 3310 is optionally transported to and from one fermentation processing station to another processing station in a multiple process production. In this illustrated example, transportable container frame 3350 transports array of sample vessels 3310 into a temperature controlled area 3311 such as a water bath. In this embodiment, temperature controlled area 3311 includes water bath 3340 in water bath container 3316, which is controlled by water bath temperature controller 3320 and temperature coil 3330 immersed in water bath 3340.

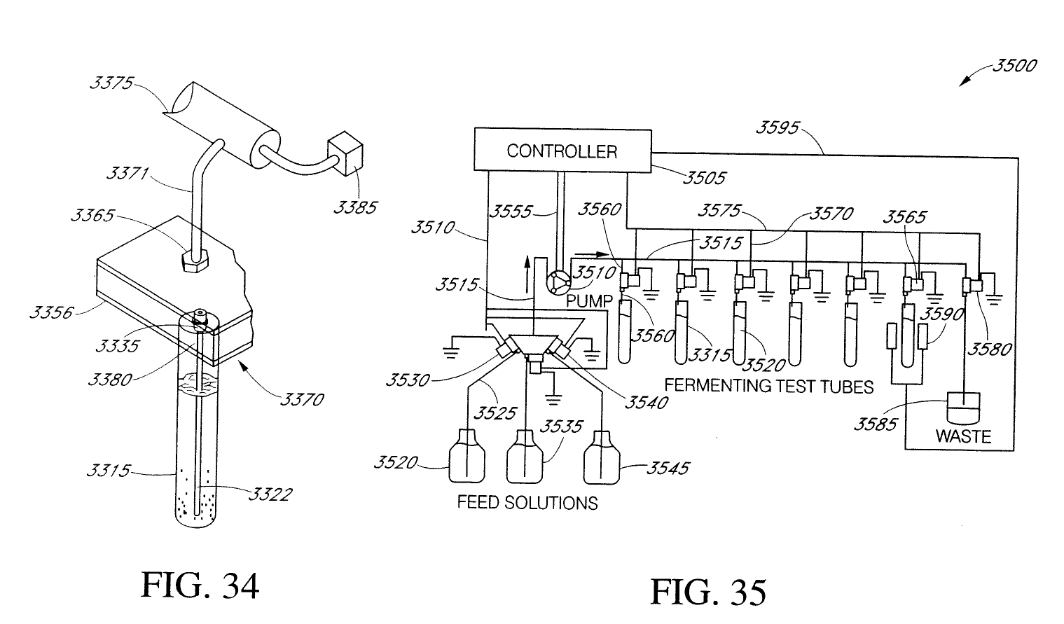

In FIGS. 33 and 34, an example gas distribution arrangement is shown. Gas distribution arrangement 3370 is comprised of gas source 3385 connected to manifold 3375. Conduit 3371 connects manifold 3375 to connector 3365. Connector 3365 connects manifold 3375 to gas distributor 3356.

In the embodiment illustrated in FIGS. 33 and 34, cannula assembly 3380 includes cannula array 3321, which include individual cannulas 3322 that correspond to sample vessel array 3310. Each individual cannula 3322 is optionally connected by a fastener 3335, which couples cannula 3322 to a gas distribution arrangement 3370. Cannula 3322 typically extends substantially to the bottom of each individual sample vessel 3315 in order to increase aeration and mixing.

FIG. 35 illustrates an example of an automated fermentation station. Process controller 3505 monitors and controls various components of station 3500 and typically is a programmable computer with an operator interface. Alternatively, process controller 3505 is any suitable processor that coordinates multiple components of station 3500, such as timing mechanisms, adding solutions, adjusting temperature, adjusting gas flow rates and gas mixtures, detecting measurements, and/or sending an alarm or notification prompting operator intervention. Electronic couples 3510, 3555, and 3595 connect various components of fermentation station 3500 to process controller 3505. For example electronic couple 3510 enables controller 3505 to start, stop, and monitor solution flow from feed solutions 3520, 3535, and 3545. Likewise, electronic couple 3575 enables controller 3505 to start, stop and monitor reagent dispensing into sample vessels 3315. Electronic couple 3595 also enables controller 3505 to transmit and receive information from sensors 3590 as well as monitor and adjust temperature controlled areas. Other coupling devices are also optionally used.

In one embodiment of fermentation apparatus 3500, feed solutions 3520, 3535, and 3545 are pumped (either singly, in combination, sequentially, or collectively) from individual feed tubes 3525 into dispensing tube 3515. Selecting the appropriate solenoid determines which feed solution is pumped through dispensing tube 3515. For example, solenoid 3530 controls flow from feed solution 3520 through feed tube 3525. In another application, a mixture of feed solutions 3520 and 3535 are simultaneously pumped into dispensing tube 3515. In another application, feed solution 3520 is fed into dispensing tube first, followed by an incubation period (directed by controller 3505), followed by feed solution 3535 being pumped into dispensing tube 3515. Different combinations of feed solutions are optionally used and more or fewer feed solutions may be used with station 3500 according to any desired application.

Using pump 3510, which is optionally a peristaltic pump, dispensing tube 3515 transfers feed solution to an individual dispensing tube 3560. Each individual dispensing tube 3560 corresponds to an individual sample vessel 3315 and tube 3560 is positioned such that feed solution 3520, for example, is transferred volumetrically from dispensing tube 3560 into its corresponding sample vessel 3315 once solenoid 3565 is opened. Each solenoid 3565 corresponds to an individual sample vessel 3315. Volumetric dispensing of feed solutions is controlled by process controller 3505 which typically controls the amount, the rate and the time of dispensing. Dispensing tube 3560 is optionally composed of plastic, metal, or any material that is non-reactive to the feed solution being dispensed.

In one embodiment, delivery solenoids 3565 work in conjunction with pump 3510 and controller 3505 to deliver multiple feed solutions such as feed solutions 3520, 3535, and 3545 into individual sample vessels 3315. Each solenoid 3565 corresponds to a sample vessel 3315 and the solenoids 3565 are manifolded together and fed by the output of a single peristaltic pump 3510. Each solenoid 3565 preferably opens sequentially in order to dispense a volumetric amount of feed solution 3520. However, parallel addition is also contemplated within the present invention.

In one embodiment, feed solution 3520 introduces nutrients into fermentation medium 3520 through dispensing tube 3515 using pump 3510 and solenoid 3565 to deliver solution 3520 to individual dispensing tube 3560. After addition of feed solution 3520, solenoid 3530 is closed and solenoid 3540 corresponding to rinse solution 3545 opens. Pump 3510 delivers rinse solution 3545 through dispensing tube 3515, thereby rinsing dispensing tube 3515 with solution 3545, which is then flushed into waste container 3585. Solenoid 3580 controls flow from dispensing tube 3515 into waste container 3585. Feed solution 3535 is then pumped through dispensing tube 3515 and dispensed through tube 3560. Dispensing tube 3515 is rinsed again with rinse solution 3545 before another addition. Solenoids 3565 are typically located very near to dispensing tube 3560 in order to minimize dead volume downstream. In this way, dispensing tube 3515 accurately delivers a known amount of feed solution 3520 and 3535 without cross contaminating or fouling the next or different addition of feed solution through dispensing tube 3515. Accordingly, each addition is volumetrically precise with a minimal, known amount of feed solution from a previous addition diluting the next addition. In this way, feed solutions such as additional nutrients, trace minerals, vitamins, sugars, carbohydrates, nitrogen containing compounds, evaporating liquids, pH balancing compounds, buffers, and other liquids may be added to fermentation media 3520 in an automated, yet highly precise manner.

Coordinated by process controller 3505, various components may be activated either at pre-determined time intervals or in response to the measurement of some physical property within sample vessel 3315. For example, in one embodiment, an operator programs process controller 3505 to incubate sample vessels 3315 for a pre-determined time period at a particular temperature, add a desired amount of feed solution 3520, and incubate further for another pre-determined time period at a different temperature. Any suitable combination of fermentation conditions may be programmed into process controller 3505, which optionally comprises a computer, computer network, other data input module, or the like.

In some embodiments, process controller 3505 coordinates temperature control, the addition of feed solutions, adjustment of gas rates and gas mixtures, incubation periods, and rinsing in response to data received from sensors 3590. Sensors 3590 are optionally located inside or outside of individual sample vessels 3315. Sensors 3590 can detect color changes spectrophotometrically, monitor evaporation rates, measure changes in optical density, detect light changes photometrically, detect pH changes, electrolytically measure redox potentials, monitor temperature fluctuations, or detect other physical changes and transmit this data to process controller 3505. In response, process controller 3505 accordingly adjusts various components of station 3500. For example, by measuring the redox potential, sensors 3590 detect when a fermentation sample is being over-oxygenated or over-provided with another gas and process controller 3505 accordingly adjusts the gas flow or gas mixture ratio. As another example, process controller 3505 can respond to a change in pH, as detected by sensors 3590, by adding a pH buffer from feed solution 3520. In one embodiment, maximum protein expression may be detected by monitoring light emission, at which point fermentation is halted to minimize wasting fermentation resources after optimum fermentation yield has been reached.

Because of the uniformity of each fermentation medium 3520, cannula 3322, and dispensing of feed solutions 3520, very few, for example, one, sensor 3590 is all that is necessary to monitor the entire array of sample vessels 3310. Alternatively, when sample vessels 3315 contain different fermentation media 3520 or undergo different fermentation conditions, numerous sensors 3590 are optionally employed.

Exemplary fermentors that are optionally adapted for use in the systems of the present invention are also described in, e.g., U.S. Patent Publication No. 2002/0146818, entitled “MULTI-SAMPLE FERMENTOR AND METHOD OF USING SAME,” filed Feb. 8, 2002 by Downs et al., U.S. Pat. No. 6,723,555, entitled “MULTI-SAMPLE FERMENTOR AND METHOD OF USING SAME,” filed Feb. 8, 2002 by Downs et al., and U.S. Pat. No. 6,635,441, entitled “MULTI-SAMPLE FERMENTOR AND METHOD OF USING SAME,” filed Feb. 8, 2001 by Downs et al., which are each incorporated by reference.

J. Centrifuges

The systems of the invention optionally include centrifuges or centrifugation stations either outside of or within a given work perimeter. These stations are typically used to harvest or concentrate cells, e.g., as part of a target protein isolation process or another application. Automated centrifuges that can be adapted for use in the systems of the invention are also described in, e.g., U.S. Patent Publication No. 2002/0132354, entitled “AUTOMATED CENTRIFUGE AND METHOD OF USING SAME,” filed Feb. 8, 2002 by Downs et al., which is incorporated by reference.

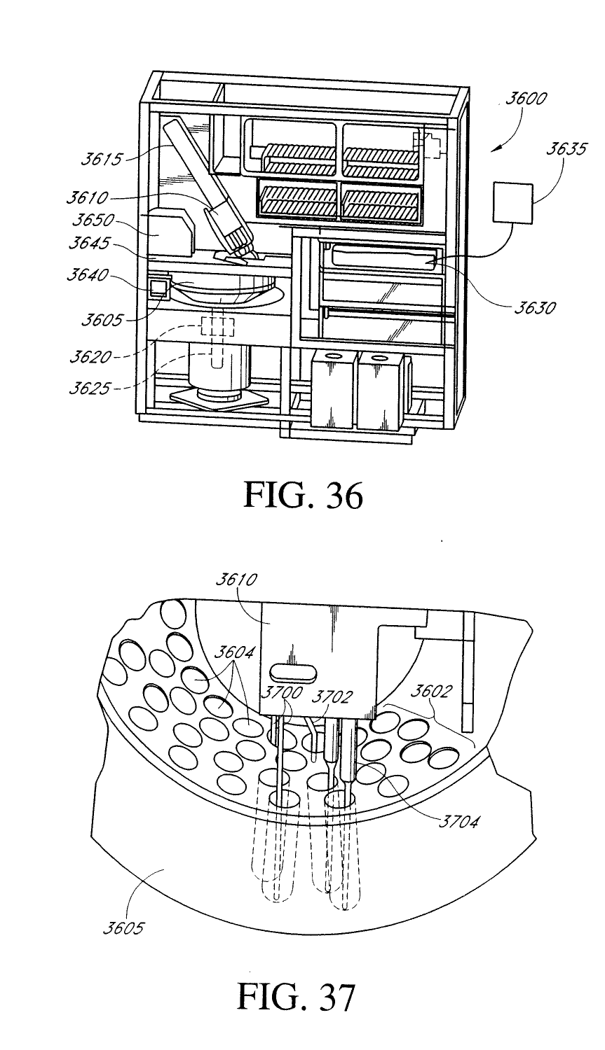

To further illustrate, FIGS. 36-41 schematically show an embodiment of automated centrifuge station 3600 that is optionally included in the systems of the invention. In this embodiment, the automated centrifuge station 3600 includes large rotor 3605 containing a plurality of clusters 3602 of cavities or holes 3604 arranged to cooperate with aspirate tubes 3700, dispense tubes 3702 and rods 3704, shown in FIG. 37. Tubes 3700 and 3702 and rods 3704 are mounted on moveable head 3610 that rides on track 3615. Moveable head 3610 can position tubes 3700 and 3702 and rods 3704 into or adjacent to cavities 3604. When inserted into cavities 3604, aspirate tubes 3700 can aspirate fluids from one cluster 3602 of cavities 3604 while rods 3704 sonicate fluid in second cluster 3602 of cavities 3604. Dispense tubes 3702 are arranged to dispense fluid into the second cluster of cavities. In some embodiments, the aspiration and sonication operations can occur substantially simultaneously. The aspiration, sonication and dispense operations can be performed substantially simultaneously, or in any order necessary to efficiently process fluid samples. In this manner, the efficient automated processing of a large number of discrete fluid samples can be performed without substantial human intervention.

Automated centrifuge station 3600 also employs rotor position sensor 3620. In some embodiments, rotor position sensor 3620 is a rotary optical encoder. Other types of devices used for measuring the rotation and position of rotor shaft 3625 can be employed, such as inductive angle measuring devices, resolvers and other similar apparatus. Rotor position sensor 3620 is positioned on rotor shaft 3625 and communicates with controller 3630 which is operated through operator interface 3635. Certain available controllers or controller components can be used to direct rotor positioning and/or centrifugation by a rotor motor, e.g., the 2400 modular performance AC drive available, e.g., from UNICO, Inc. (Franksville, Wis., USA). The operator interface allows a technician to program the controller with a “recipe” which is a list of instructions that tells the controller to perform specific functions appropriate to a specific task. For example, a component such as a protein that is suspended in a fluid may need to be isolated through a centrifugation process. The technician programs the appropriate “recipe” into the controller and then proceeds to load vessels into large rotor 3605.

Referring to FIG. 36, once a recipe has been entered through operator interface 3635 and into controller 3630, the controller determines the position of rotor 3605 through rotor position sensor 3620. The technician inserts vessels into cavities 3604 and then places both hands on the switch 3640. The rotor is then rotated, presenting a new cluster 3602 of cavities 3604 for loading. Switch 3640 provides an important safety feature by forcing the technician to place his hands on the switch before the rotor is rotated. This avoids any possible injury to the technician, by keeping his hands well away from the rotating rotor. In certain embodiments, switch 3640 comprises one or more touch buttons. Touch buttons register an operator's touch, converting that touch into an electrical output that signals the controller to rotate the rotor. Other types of safety switches such as capacitive and photoelectric sensors and other suitable devices can be employed in place of the switch. Ordinarily, there are two touch buttons, i.e., one for each of an operator's hands. Thus, an operator places two hands on the touch buttons, ensuring that the operator's hands are out of any danger from the rotor before engaging the rotor.

After placement of vessels into cavities 3604, rotor cover 3645 is positioned over rotor 3605. Rotor 3605 is then spun, separating the different components through a centrifugation process. When the centrifugation process is complete, rotor 3605 is stopped. Controller 3630 then instructs rotor cover 3645 to slide away, revealing rotor 3605.

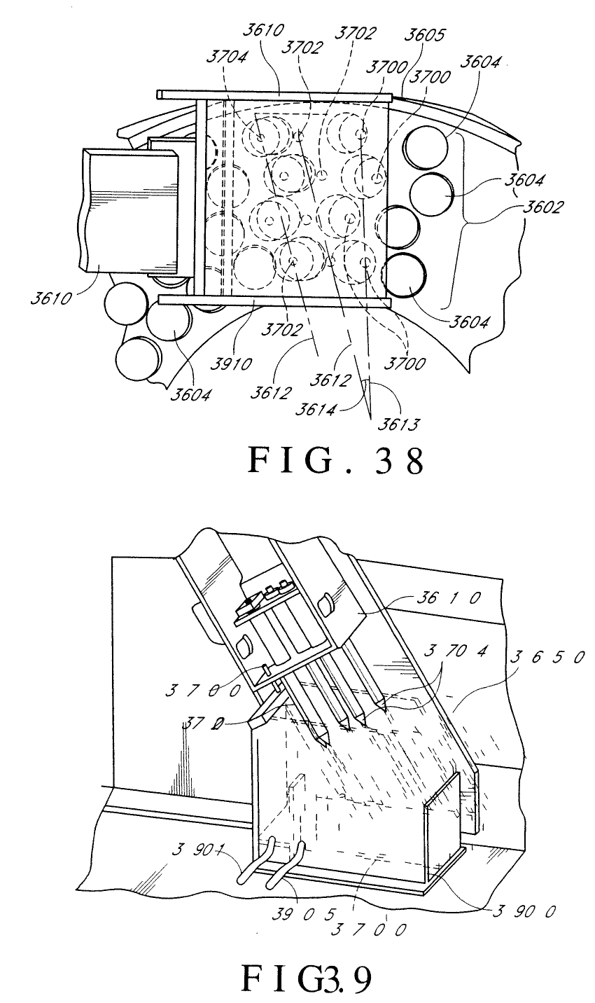

Referring to FIGS. 37 and 38, the insertion of the aspirate tubes 3700, dispense tubes 3702, and rods 3704 into cavities 3604 will now be described. In one embodiment, rotor 3605 contains ninety-six cavities 3604 arranged in twenty-four clusters 3602 of four cavities 3604. As shown in FIG. 38, the cavities are arranged substantially radially on rotor 3605. The longitudinal axes of all of the cavities of each cluster are substantially parallel, thereby permitting the substantially simultaneous insertion of one or more of the rods, aspirate tubes and/or dispense tubes.

Referring to FIG. 38, one arrangement of rods 3604 and aspirate tubes 3700 and dispense tubes 3702 is illustrated. Four aspirate tubes, four dispense tubes and four rods are mounted on movable head 3610. In one embodiment, the dispense tubes and rods have parallel tube axes 3612. The aspirate tubes are arranged on a tube axis 3613 that is angled 3614 relative to the dispense tube axis. The angle allows the aspirate tubes and rods to be substantially simultaneously inserted into two adjacent clusters 3602. This allows the aspiration of fluids from one cluster 3602 of cavities 3604 and the simultaneous sonication of an adjacent cluster of cavities. Shown in FIG. 37, the dispense tubes are significantly shorter than the aspirate tubes 3700 and can be arranged to dispense fluid into the same cavities that the rods are positioned in. Other arrangements of aspirate tubes and dispense tubes and cavities can be constructed, such as positioning tubes 3700 and rods 3605 in a splayed arrangement so that three or more clusters 3602 of cavities 3604 can be substantially simultaneously serviced.

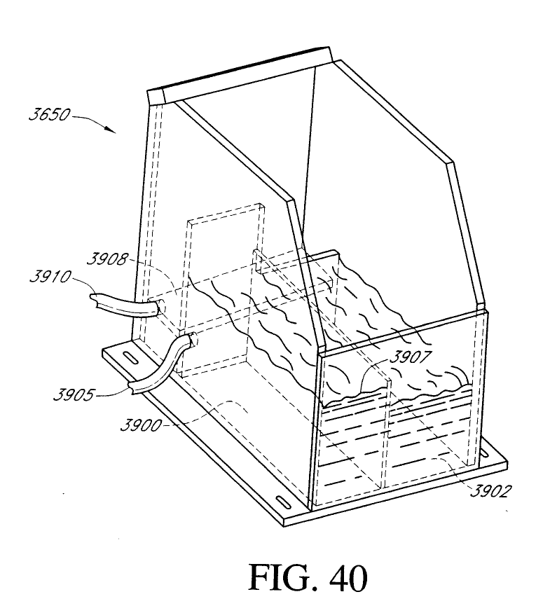

Referring to FIGS. 39 and 40, waste/rinse container 3650 is illustrated. After tubes 3700 and 3702 and rods 3704 have performed their functions in cavities 3604, rotor cover 3645 is slid over rotor 3605. This positions the waste/rinse container under movable head 3610. The moveable head is then transported down track 3615 and tubes 3700 and 3702 and rods 3704 are positioned in the waste/rinse container. Aspirate tubes 3700 are inserted into tube bin 3900 with rods 3704 inserted into rod bin 3902. Dispense tube 3702 does not need rinsing, as it does not need to contact fluids or other substances in the cavities. Fluid source 3655 delivers fluid through rinse fluid input 3905 and into tube bin 3900. Rinse fluid 3907 can be dionized water, alcohol, detergent, or any other suitable rinsing fluid. Rinse fluid 3907 washes aspirate tube 3700 and, if necessary, aspirate tubes 3700 can aspirate rinse fluid 3907 and dump it into waste dump 3660. The rinse fluid fills the tube bin and then overflows into rod bin 3902 where it rinses sonication rod 3704. Dispense tube 3702 can dispense fluids into rinse fluid 3907, which then runs down run-off ramp 3908 to rinse fluid exit 3910 and to waste dump 3660 through tubes or other means that are not illustrated.

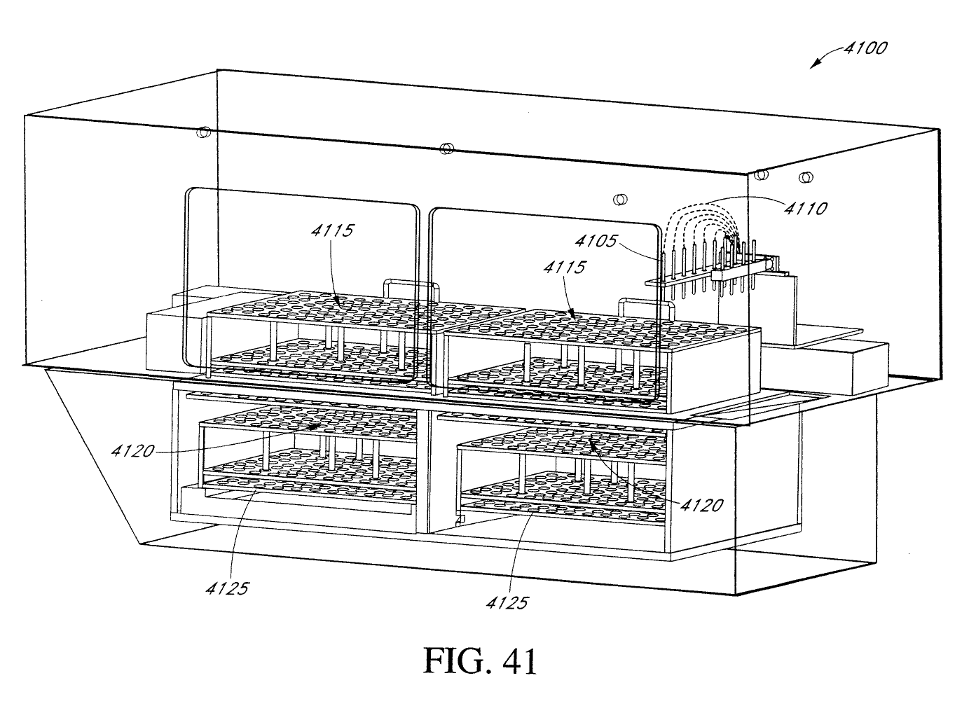

Referring to FIG. 41, fraction collector 4100 is illustrated. Fraction collector 4100 is structured to collect sample components that have been isolated during a centrifugation process. Tips 4105, that are connected to hoses 4110, deposit isolated material obtained from cavities 3604 by aspirate tubes 3700 into filter bed 4115, typically arranged in a standard 96, 384, or 1536 member sample format. The fraction collector optionally comprises one or more additional tips or sets of tips that dispense fluid from sources other than the cavities. Hoses 4110 communicate with aspirate tubes 3700 as described above. In one embodiment, filter bed 4115 comprises a plurality of vessels, each comprising a filter structured to remove particles that have not been separated during the centrifugation process. For example, nitrocellulose filters or Whatman filters or sepharose resin filters or other suitable filters can be employed.

After passing through filter bed 4115, the fluid then drops down onto resin bed 4120, which typically is arranged in standard 96, 384, or 1536 member sample format. Resin bed 4120 is structured to catch the components that have been isolated during the centrifugation process. For example, proteins that have passed through the filter bed 4115 are now caught in resin bed 4120. In one embodiment, a nickel chelate resin is employed, but other types of resins, such as ion-exchange resins and hydrophobic interaction resins, can be employed. Located beneath resin bed 4120 is catch tray 4125 that catches any remaining fluids and deposits them in waste dump 3660.

Also shown in FIG. 36 is controller 3630. As discussed above, the controller optionally comprises a general purpose computing device that controls a function of automated centrifuge 3600. In one embodiment, the automated centrifuge employs a controller that comprises two programmable logic controllers (PLCs) with one PLC operating operator interface 3635 and directing the second PLC to perform the variety of functions of the automated centrifuge 3600. In an alternate similar embodiment, one PLC controls the fraction collection functions for the fraction collector noted above while another controls the user interface, the main rotor functions, and, optionally, controls the PLC that controls the fraction collector functions. The number, function and arrangement of PLC can vary, depending on the system components and the operations that the overall system performs.

Once sample fractions are collected in, e.g., microtiter plates, collection tubes, or the like, the samples are then typically subjected to additional downstream processing, such as crystallization and structural analysis as desired.

K. Sample Holders

The systems and methods of the present invention can be adapted for use with essentially any type of sample holders or containers. Typical sample holders or containers used in the systems of the invention include containers, substrate surfaces, and the like. Exemplary containers include multi-well containers, such as micro-well plates, cell culture flasks, reaction blocks, and other containers used, e.g., to perform multiple assays, synthesis reactions, or other processes in parallel. Multi-well containers such as these typically include, e.g., 6, 12, 24, 48, 96, 192, 384, 768, 1536, or more wells, and are generally available from various commercial suppliers including, e.g., Greiner America Corp. (Lake Mary, Fla., USA), Nalge Nunc International (Rochester, N.Y., USA), H+P Labortechnik A G (Oberschleiβheim, Germany), and the like. Additional details relating to reaction blocks that are suitable for use in the systems of the invention are provided in, e.g., U.S. Pat. No. 6,682,703, entitled “PARALLEL REACTION DEVICES,” filed Sep. 5, 2001 by Micklash I I, et al., which is incorporated by reference. Cell culture containers or flasks (e.g., Corning® RoboFlask™ Cell Culture Vessels, etc.) are commercially available from, e.g., Corning, Inc. Life Sciences (Acton, Mass., USA).

To further illustrate, the systems of the invention are also optionally configured to dispense fluidic materials on substrate surfaces. For example, the systems described herein can be utilized to produce dot arrays or the like on substrate surfaces at various different densities. Arrayed materials are commonly used in, e.g., clinical testing (e.g., blood cholesterol tests, blood glucose tests, pregnancy tests, ovulation tests, etc.) in addition to many other applications known in the art. Essentially any substrate material is optionally adapted for use with the systems of the invention. In certain embodiments, for example, substrates are fabricated from silicon, glass, or polymeric materials (e.g., glass or polymeric microscope slides, silicon wafers, etc.). Suitable glass or polymeric substrates, including microscope slides, are available from various commercial suppliers, such as Fisher Scientific (Pittsburgh, Pa., USA) or the like. Optionally, substrates utilized in the systems of the invention are membranes. Suitable membrane materials are optionally selected from, e.g. polyaramide membranes, polycarbonate membranes, porous plastic matrix membranes (e.g., POREX® Porous Plastic, etc.), porous metal matrix membranes, polyethylene membranes, poly(vinylidene difluoride) membranes, polyamide membranes, nylon membranes, ceramic membranes, polyester membranes, polytetrafluoroethylene (TEFLON™) membranes, woven mesh membranes, microfiltration membranes, nanofiltration membranes, ultrafiltration membranes, dialysis membranes, composite membranes, hydrophilic membranes, hydrophobic membranes, polymer-based membranes, a non-polymer-based membranes, powdered activated carbon membranes, polypropylene membranes, glass fiber membranes, glass membranes, nitrocellulose membranes, cellulose membranes, cellulose nitrate membranes, cellulose acetate membranes, polysulfone membranes, polyethersulfone membranes, polyolefin membranes, or the like. Many of these membranous materials are widely available from various commercial suppliers, such as, P.J. Cobert Associates, Inc. (St. Louis, Mo., USA), Millipore Corporation (Bedford, Mass., USA), or the like.

In some embodiments, sample holders are labeled with at least one identifier or label, for example, a bar code, RF tag, color code, or other label. When the sample holders are labeled with a bar code, each robot is typically provided with a bar code reader. The bar code readers are optionally positioned on the robotic arms or any other position on the robot depending upon the application and type of sample container used. By identifying each specimen plate with a bar code, RF tag, or color code, the system can positively identify each sample holder, e.g., when retrieving, processing, or detecting each sample. In addition, the information is also optionally used to provide reports regarding assay outcomes and results, and to provide an inventory of a large number of samples, e.g. libraries of nucleic acid samples. For example, an inventory is optionally used to compare a list of desired plates with a list of plates present in the system, and notify an operator of any discrepancies.

In certain embodiments, when a sample holder is provided with a bar code at opposite ends, and the bar codes have indicia relating orientation, the systems of the present invention determine which end of the sample holder is facing the robot. For example, one end of the sample holder optionally has a bar code with an even code, while the opposite end of the sample holder has an odd numbered code. Accordingly, the robots used in the systems of the invention easily determine whether a leading or trailing edge of a sample holder is facing the bar code reader in the robot. In this manner, the robot reliably and consistently determines which end of a sample holder to insert into each device.

L. Controllers

The compound profiling systems of the invention also typically include controllers that are operably connected to one or more components (e.g., automated cell passaging stations, incubation devices, dispensing device, robotic gripping devices, assaying components, etc.) of the system to control operation of the components. More specifically, controllers are generally included either as separate or integral system components that are utilized, e.g., to rotate rotational mechanisms of cell culture dissociators, to move robotic gripping devices, to regulate quantities of samples, reagents, cleaning fluids, or the like dispensed from dispense heads, the movement of pushers, the movement of translocation mechanisms, etc. Controllers and/or other system components is/are optionally coupled to an appropriately programmed processor, computer, digital device, or other information appliance (e.g., including an analog to digital or digital to analog converter as needed), which functions to instruct the operation of these instruments in accordance with preprogrammed or user input instructions, receive data and information from these instruments, and interpret, manipulate and report this information to the user.

Any controller or computer optionally includes a monitor that is often a cathode ray tube (“CRT”) display, a flat panel display (e.g., active matrix liquid crystal display, liquid crystal display, etc.), or others. Computer circuitry is often placed in a box, which includes numerous integrated circuit chips, such as a microprocessor, memory, interface circuits, and others. The box also optionally includes a hard disk drive, a floppy disk drive, a high capacity removable drive such as a writeable CD-ROM, and other common peripheral elements. Inputting devices such as a keyboard or mouse optionally provide for input from a user.

The computer typically includes appropriate software for receiving user instructions, either in the form of user input into a set of parameter fields, e.g., in a GUI, or in the form of preprogrammed instructions, e.g., preprogrammed for a variety of different specific operations. The software then converts these instructions to appropriate language for instructing the operation of one or more controllers to carry out the desired operation, e.g., varying or selecting the rate or mode of movement of various system components, directing translation of robotic gripping devices, fluid dispensing heads, or of one or more multi-well containers or other vessels, or the like. The computer then receives the data from, e.g., sensors/detectors included within the system, and interprets the data, either provides it in a user understood format, or uses that data to initiate further controller instructions, in accordance with the programming, e.g., such as in monitoring incubation temperatures, detectable signal intensity, or the like.

To illustrate, the systems of the invention generally include scheduler software that keeps track of databases and process schedules. Exemplary processes that can be performed using this software include passaging, expansion, profiling, and harvesting. More specifically, the software utilized to control the operation of the automated cell passaging stations of the systems described herein typically includes logic instructions that direct, e.g., translational mechanisms and multicontainer holders to translate cell culture dissociators to selected positions along translational axes, rotational mechanisms to rotate container holders at selected rates, material handling component to dispense material into, and/or to remove material from, cell culture containers, container holders to move to closed positions or to open positions. In certain applications, after cell lines have been expanded to desired quantities in separate cell culture containers, the cells are pooled for dispensing into multi-well containers for assaying or other processing. In these embodiments, system software typically includes logic instructions that direct, e.g., fluidic material transfer components to pool separate first cell culture media from m first cell culture containers in n second containers to produce pooled cell culture media (where m is an integer greater than one and n is an integer greater than zero and less than m), and the fluidic material transfer components to transfer selected volumes of the pooled cell culture media from the n second containers into selected wells of p multi-well containers (where p is an integer greater than one). In this manner, substantially uniform concentrations of cells are dispensed into each well of the multi-well containers. To further illustrate, system software also typically includes logic instructions that direct, e.g., the movement of pin tools between test reagent source regions and assaying regions of assaying components, the attachment and/or detachment of pin tools to or from chassis of assaying components, etc.

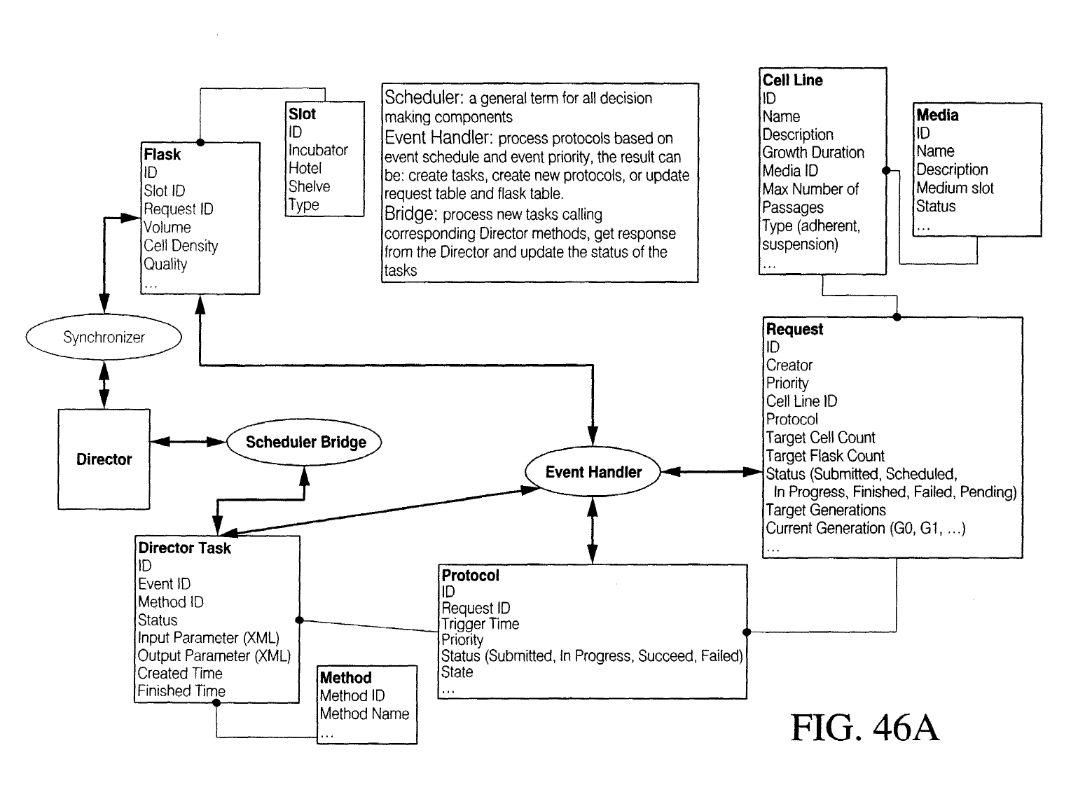

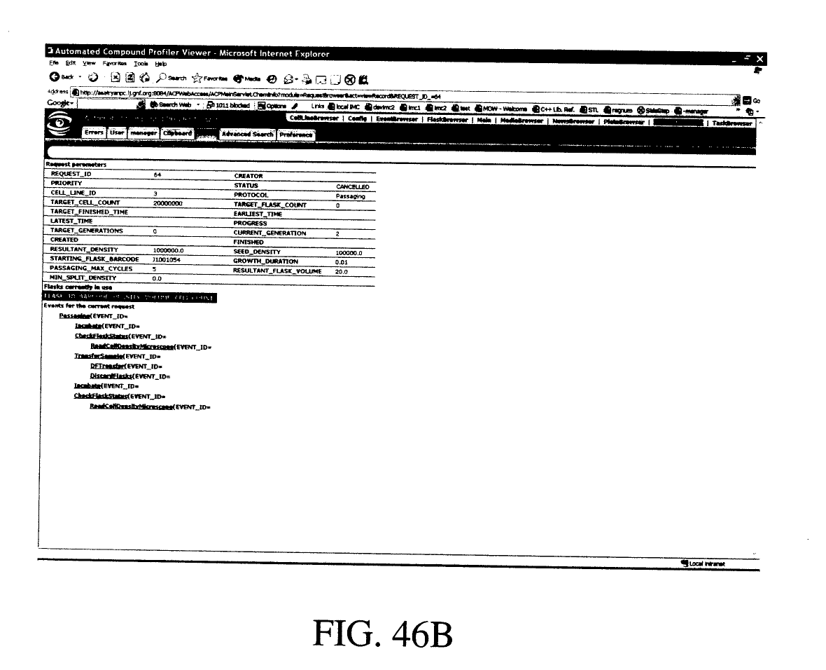

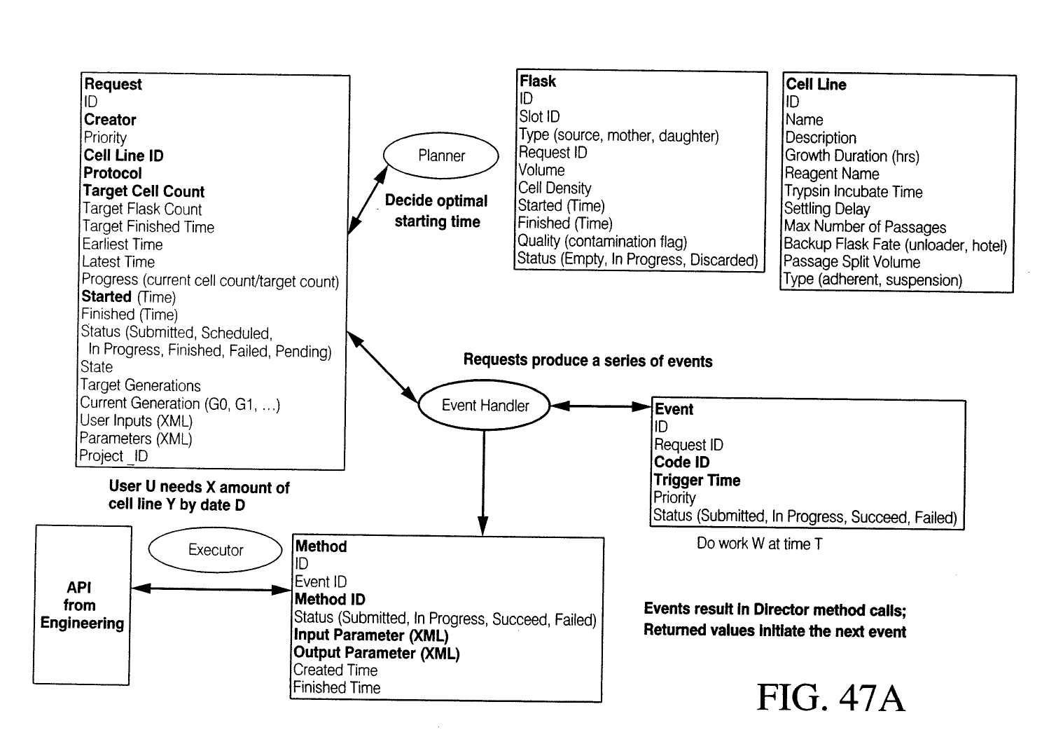

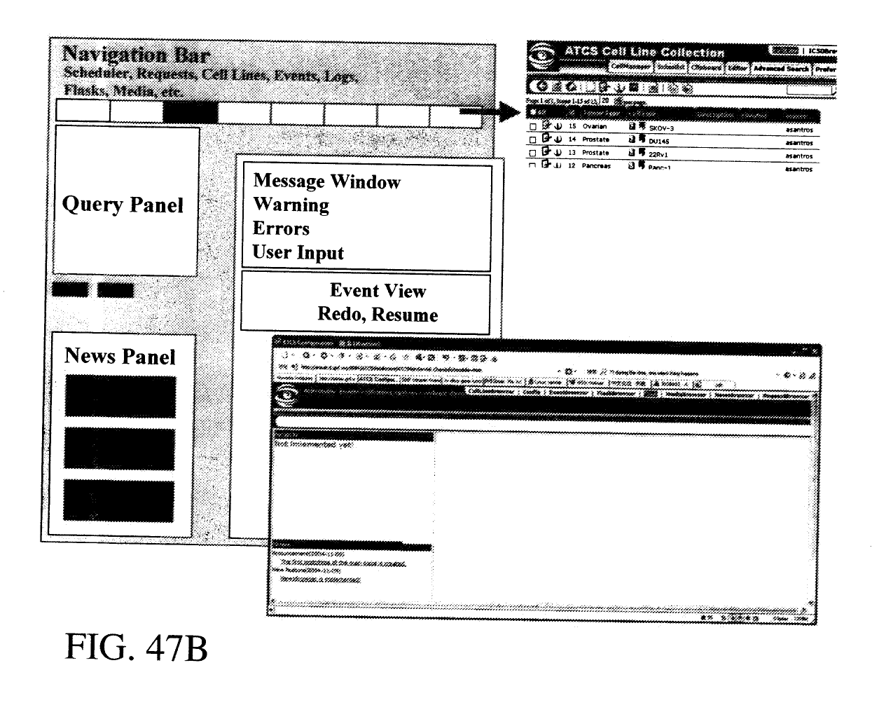

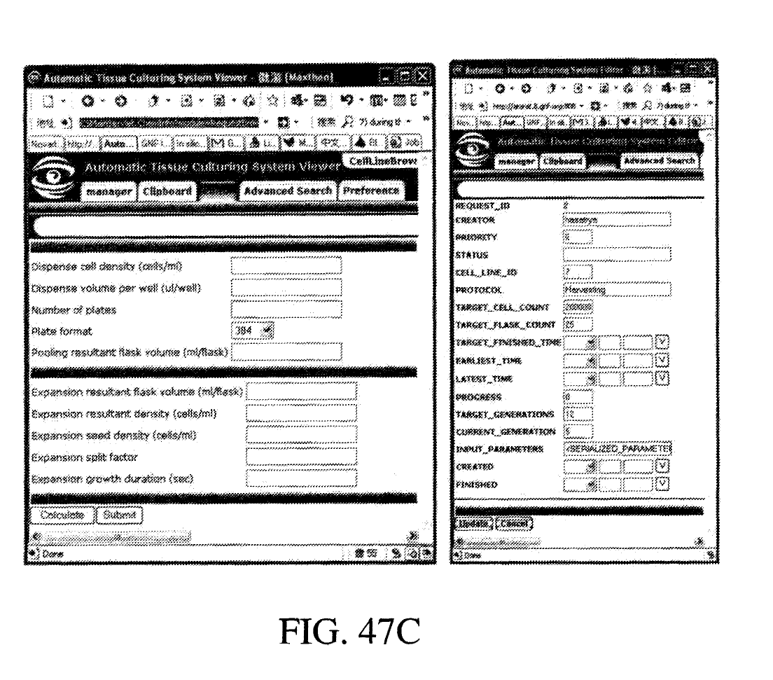

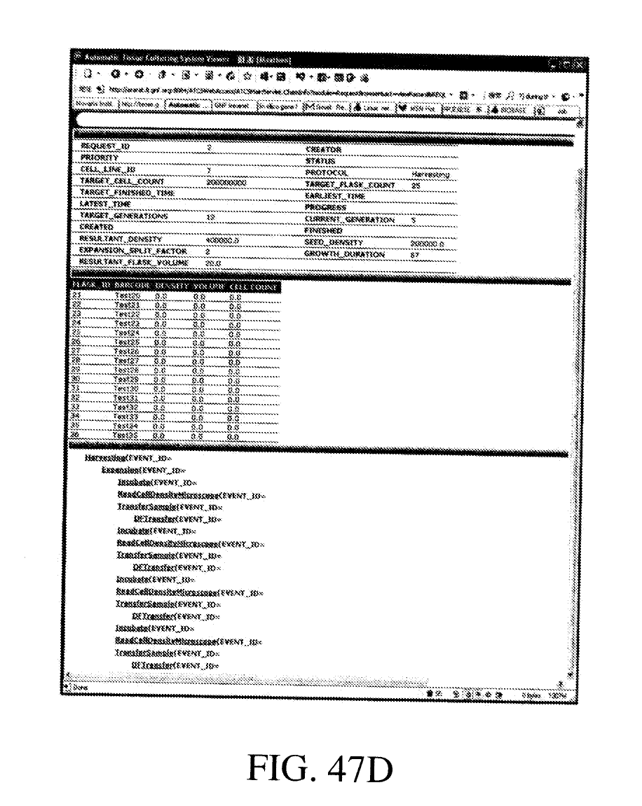

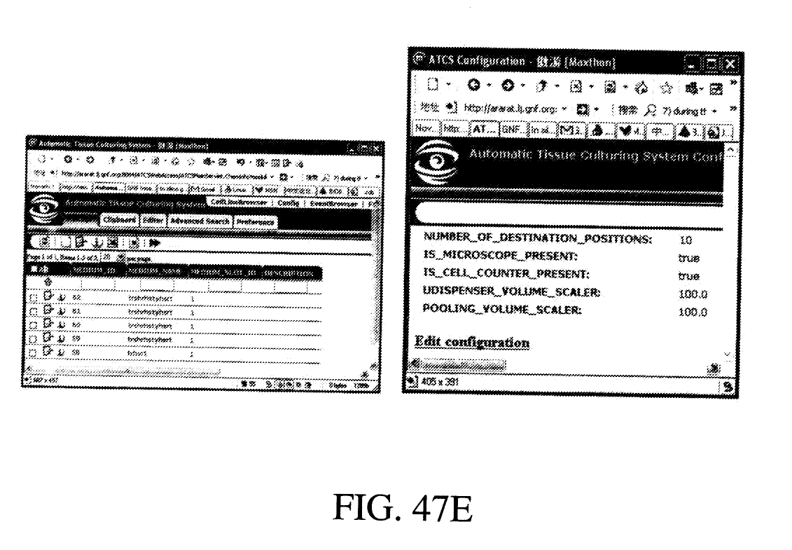

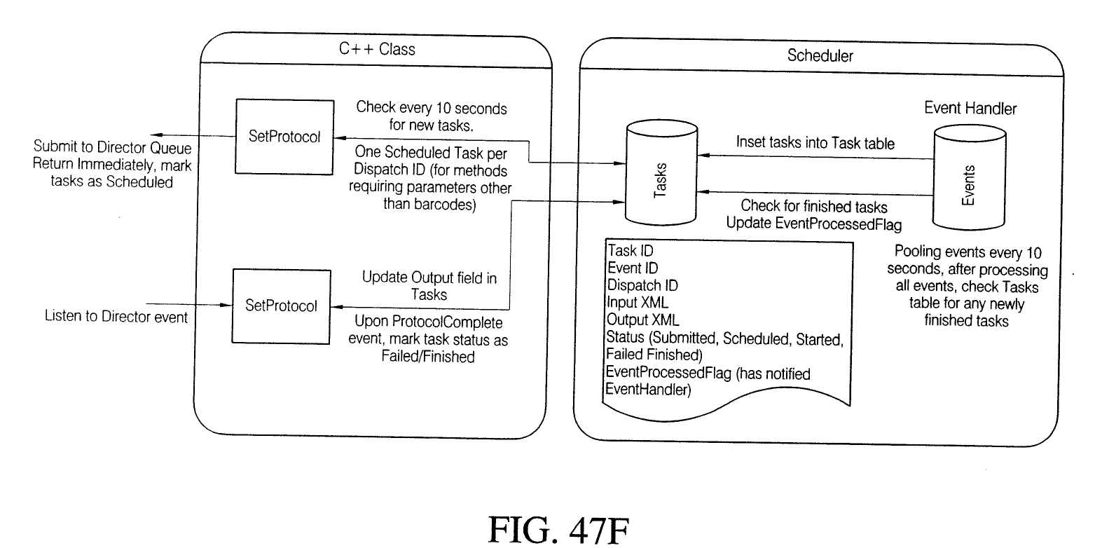

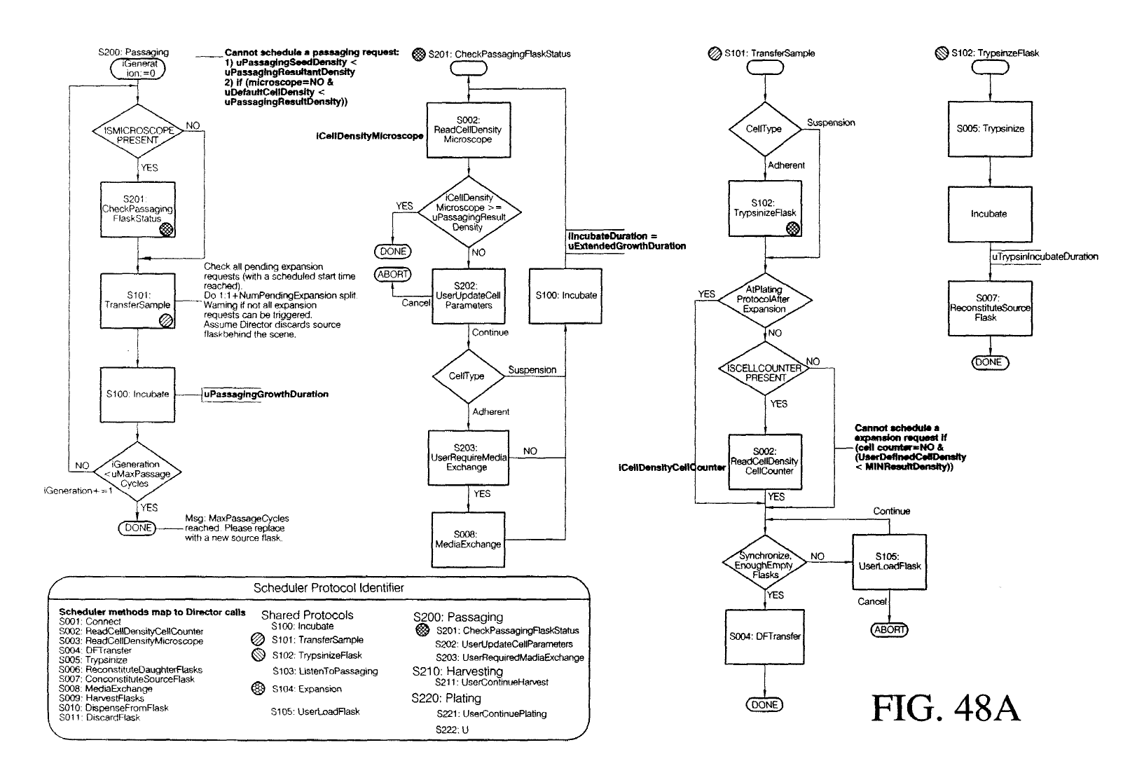

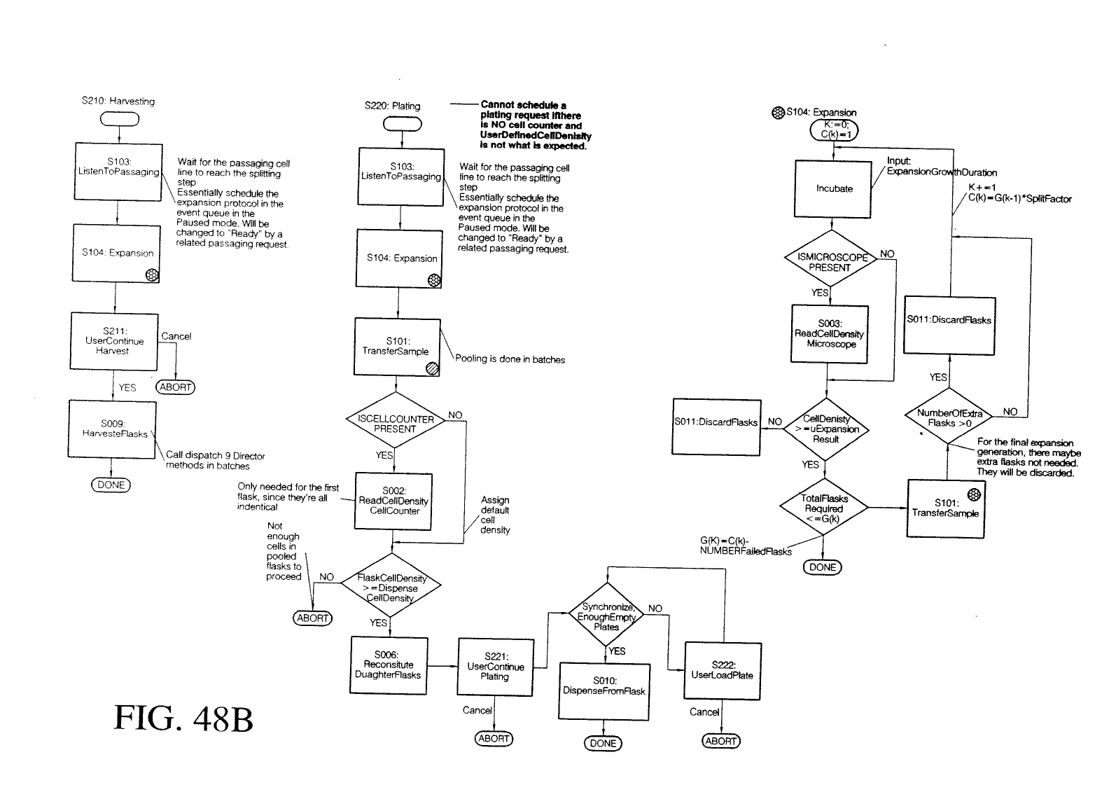

To further illustrate, FIGS. 46-48 schematically show aspects related to various exemplary embodiments of control software utilized in the systems of the present invention. More specifically, FIG. 46A is a flow chart illustrating aspects of control software architecture according to one embodiment of the invention. FIG. 46B shows a display screen related to the control software architecture shown in FIG. 46A. Other exemplary aspects embodied in this software architecture, in certain embodiments, include, e.g., cell expansion protocols (e.g., harvesting, plating, etc.), planning modules, flask/slot web interfaces, support for multiple microscope measurement types, modules to collect user feedback, etc. In addition, FIG. 47A is a flow chart illustrating aspects of control software architecture according to specific embodiments of the invention. FIG. 47B schematically shows an interface of the control software depicted in FIG. 47A according to one embodiment of the invention. FIG. 47C show display screens for submitting requests that are related to the control software architecture depicted in FIG. 47A according to one embodiment of the present invention. FIG. 47D shows a display screen for monitoring requests (report view) that are related to the control software architecture depicted in FIG. 47A according to one embodiment of the present invention. FIG. 47E show display screens depicting various exemplary operator tools that are related to the control software architecture depicted in FIG. 47A according to one embodiment of the present invention. FIG. 47F shows a diagram that depicts certain software component interfaces with engineering director software (e.g., method calls, return event processing, refresh flask inventory, etc.) that are related to the control software architecture depicted in FIG. 47A according to one embodiment of the present invention. In certain embodiments, for example, systems are controlled by director software that links up devices in the system in an “assay”. In the systems of the invention, a scheduler software piece is typically used to replace the user (who would typically otherwise execute the assays, at least in part, manually) in determining when and which assay to execute. As shown in FIG. 47F, in between the director and scheduler software is a program (a scheduler bridge) that links the two together in this embodiment. That is, the scheduler bridge software handles the passage of information between the director and scheduler software components. FIGS. 48 A and B are flow charts illustrating exemplary scheduler software protocols (e.g., passaging, check passaging flask status, transfer sample, trypsinize flask, harvesting, plating, expansion) according to specific embodiments of the invention.

The computer can be, e.g., a PC (Intel x86 or Pentium chip-compatible DOS™, OS2™, WINDOWS™, WINDOWS NT™, WINDOWS95™, WINDOWS98™, WINDOWS2000™, WINDOWS XP™, LINUX-based machine, a MACINTOSH™, Power PC, or a UNIX-based (e.g., SUN™ work station) machine) or other common commercially available computer that is known to one of skill. Standard desktop applications such as word processing software (e.g., Microsoft Word™ or Corel WordPerfect™) and database software (e.g., spreadsheet software such as Microsoft Excel™, Corel Quattro Pro™, or database programs such as Microsoft Access™ or Paradox™) can be adapted to the present invention. Software for performing, e.g., multi-well container positioning, fluid removal from selected wells of a multi-well container is optionally constructed by one of skill using a standard programming language such as AppleScript, Visual basic, Fortran, Basic, Java, or the like.

In certain embodiments, the bar codes described above or other markers or labels affixed to the sample holders are optionally used to provide a compound or sample plate inventory, e.g., that is tracked by a controller for the systems of the invention. The inventory typically keeps track of what samples and/or sample holders are in the system, as well as their location and status within the system. By providing a bar code system on the sample plates, the robotic arms are used to track the plates throughout the system. In addition, information can be transferred to a central controller, e.g., a PC, that coordinates locations with resulting data from various processes to provide an inventory combined with assay results. Typically, the systems include container location databases operably connected to controllers. These databases generally include entries that correspond to locations of containers in the system or other desired information.

M. System Component Fabrication

Device components or portions thereof (e.g., rotational mechanisms, container holders, retention plates, dispense heads, housings, shelves, support elements, frame components, position adjustment components, etc.) are optionally formed by various fabrication techniques or combinations of such techniques including, e.g., milling, machining, welding, stamping, engraving, injection molding, cast molding, embossing, extrusion, etching (e.g., electrochemical etching, etc.), or other techniques. These and other suitable fabrication techniques are generally known in the art and described in, e.g., Altintas, Manufacturing Automation Metal Cutting Mechanics, Machine Tool Vibrations, and CNC Design, Cambridge University Press (2000), Molinari et al. (Eds.), Metal Cutting and High Speed Machining, Kluwer Academic Publishers (2002), Stephenson et al., Metal Cutting Theory and Practice, Marcel Dekker (1997), Rosato, Injection Molding Handbook, 3^{rd} Ed., Kluwer Academic Publishers (2000), Fundamentals of Injection Molding, W. J. T. Associates (2000), Whelan, Injection Molding of Thermoplastics Materials, Vol. 2, Chapman & Hall (1991), Fisher, Extrusion of Plastics, Halsted Press (1976), and Chung, Extrusion of Polymers: Theory and Practice, Hanser-Gardner Publications (2000), which are each incorporated by reference. In certain embodiments, following fabrication, device components or portions thereof are optionally further processed, e.g., by coating surfaces with a hydrophilic coating, a hydrophobic coating (e.g., a Xylan 1010DF/870 Black coating available from Whitford Corporation (West Chester, Pa., USA), epoxy powder coatings available from DuPont Powder Coatings USA, Inc. (Houston, Tex., USA)), or the like, e.g., to prevent interactions between component surfaces and reagents, samples, or the like, to provide a desired appearance, and/or the like.

The devices of the invention are typically assembled from individually fabricated component parts (e.g., shelves, housings, frame components, etc). Device fabrication materials are generally selected according to properties, such as durability, expense, or the like. In certain embodiments, devices or components thereof, are fabricated from various metallic materials, such as stainless steel, anodized aluminum, or the like. Optionally, device components are fabricated from polymeric materials such as, polytetrafluoroethylene (TEFLON™), polypropylene, polystyrene, polysulfone, polyethylene, polymethylpentene, polydimethylsiloxane (PDMS), polycarbonate, polyvinylchloride (PVC), polymethylmethacrylate (PMMA), or the like. Component parts are also optionally fabricated from other materials including, e.g., wood, glass, silicon, or the like. In addition, components parts are typically welded, bonded, bolted, riveted, etc. to one another to form, e.g., an object storage module, a support structure, or the like.

### IV. Methods

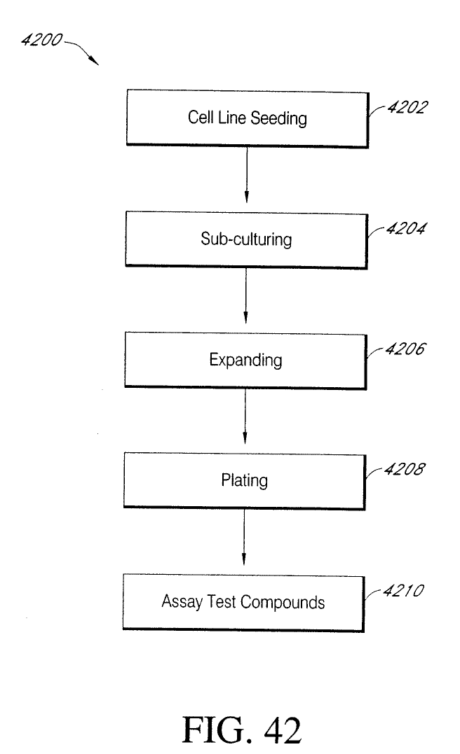

Although the systems of the invention are easily configured to perform a diverse array of applications distributed across one or more work perimeters, in some embodiments systems are configured to perform automated high throughput cell-based compound profiling assays. To illustrate, FIG. 42 is a block diagram that depicts a general method of performing a compound profiling assay according to one embodiment of the invention. As shown, compound profiling method 4200 including seeding the cell lines that are to be used to assay the test compounds or other test reagents (step 4202). As described herein, the systems of the invention also generally provide for automated storage and retrieval of cell culture flasks. Thus, following seeding step 4202, cell culture flasks are typically transferred to incubation devices for a selected incubation period using a robotic gripping device.

Method 4200 also includes automated sub-culturing or passaging of all stored cell lines (step 4204) in which robotic gripping devices transfer cell culture flasks from the incubation devices to cell culture passaging stations. This process also generally includes the occasional collection of aliquots of the cell culture media for freezing to preserve the cell lines. Cell culture passaging is typically performed to maintain the cell lines. The process generally involves splitting the cell culture in a source flask every few days to dilute the cell density and replacing old media with new media. The system also checks the source flask after an incubation period specified by the user. When the incubation period has expired, the robotic gripping device moves the source flask to a microscope for a non-intrusive (i.e., samples are not removed from the source flask) cell count. In certain embodiments, robotic gripping devices are used to shake the source flasks prior to placing the flasks at a particular station, e.g., to improve the uniformity of the concentration of cells in the flask. This step is particularly important before flasks are read on the microscope. Although the microscope typically does not provide a very accurate cell count, it does generally provide sufficient information to the scheduler to determine whether enough cells are present to proceed.

If there are not enough cells, the source flask is returned for further incubation using the robotic gripping device. If there are sufficient cells, then a sample is removed from the source flask to be analyzed on a cell counter. This will provide an accurate cell density that is used to calculate transfer volumes. The source flask will be moved proximal to a dispensing device of the cell culture passaging station positioned in the cell culture dissociator along with an empty daughter flask positioned in the multicontainer holder. If the cell culture includes adherent cells, a dissociative reagent (e.g., trypsin, etc.) is typically added to the source flask, after which the cell culture dissociator rotates the source flask to effect the dissociation of the cells as described herein. Typically, the source flask is incubated (e.g., between about 0.5 minutes and about 30 minutes) following the addition of the dissociative reagent and prior to being rotated by the cell culture dissociator. The calculated transfer volume of sample is transferred from the source flask to the daughter flask. Media is added on top of the sample in the daughter flask. The source flask is then discarded and the daughter is returned for incubation. In this process, the daughter flask now becomes the source flask and the cycle repeats after the incubation period has expired.

In addition, method 4200 also includes the automated expansion of selected cell cultures for profiling assays (step 4206). This process includes periodically cell counting to adapt the processing parameters for optimum expansion and so that all cell lines in a particular group are grown at the same time. The expansion process differs from cell culture passaging in that the process creates multiple daughter flasks depending on the needs of the selected profiling or harvesting process. The multiple daughter flasks are typically produced using cell culture passaging station, similar to the approach described above.

Once the cells are expanded to the correct quantities, the cells are then pooled for profiling. The cells are then typically dispensed into assay plates at a dispensing station that includes a dispensing device, such as the device shown in FIG. 23A (step 4248). An exemplary method of dispensing volumes with substantially uniform cell concentrations is described further below. Then, compounds and/or other reagents are typically added to the assay plates from reagent plates using a pintool of an assaying component of the system. The assay plate is typically incubated for a selected period of time, returned to the dispensing device for reagent addition, and is read using a detection component (step 4210).

In some embodiments, cells can also be harvested into a external flask. This is typically used to gather a large volume of cells. In this process, each expanded flask is positioned relative to the dispensing device of the cell passaging station and the cell culture media is aspirated from the expanded flask and dispensed into an external flask. The external flask is typically placed on hot plate at 37° C. with a magnetic stirrer. Once all the cells are collected, the cell line can be used for high throughput screening or other processes.

The invention also provides a method of dispensing substantially uniform concentrations of cells of the same line in to multi-well plates. The maximum holding capacity of certain cell culture flasks is 100 mL. When these flasks contain 10 mL they are referred to herein as “Min” flasks. In contrast, one of these flasks contains over 10 mL, it is referred to herein as a “Max” flask. In addition, multi-well plates such as 384- and 1536-well plates typically have volume capacities of about 10 mL.

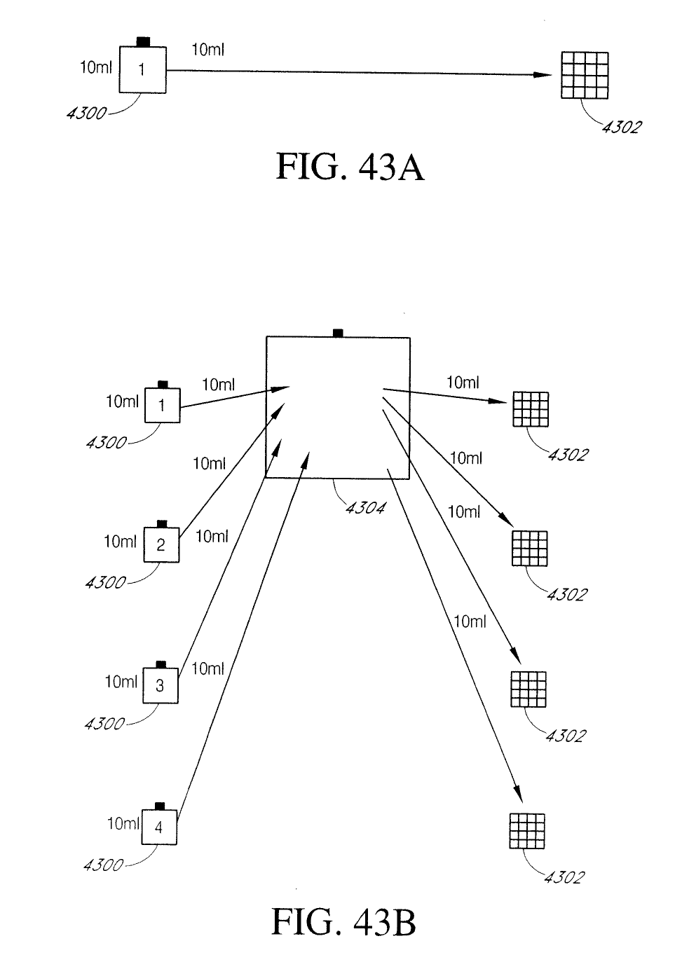

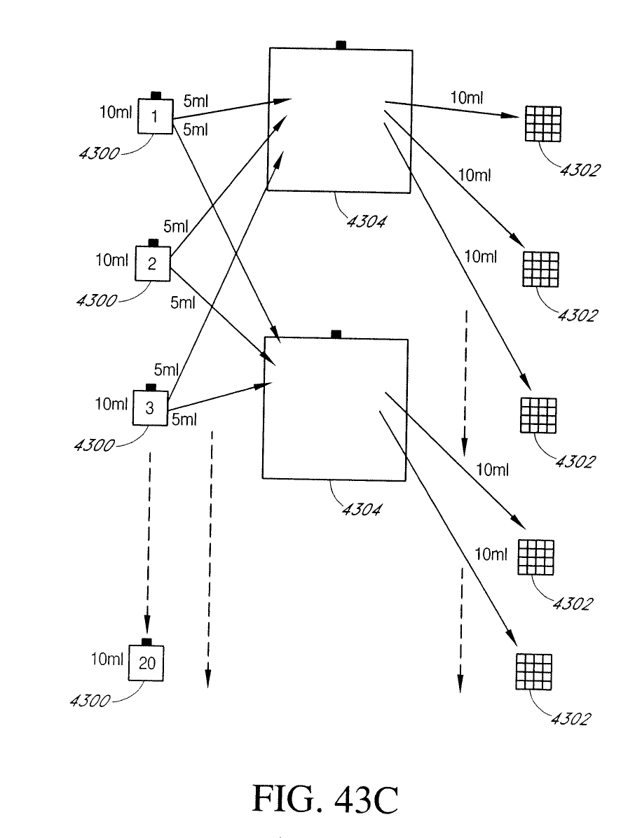

There are generally three different cases for transferring volumes with substantially uniform concentrations of cells from these flasks into multi-well plates. In the first case, one cell line is dispensed into one plate. More specifically, a sample of cells is typically drawn from one “Min” flask to determine the cell concentration. As shown in the method depicted in FIG. 43A, aliquots with cells from “Min” flask 4300 can then be transferred into one multi-well plate 4302. That is, one “Min” flask 4300 containing 10 mL of cell culture can be dispensed into a multi-well plate 4302 having a volume capacity of 10 mL.

In the second case, one cell line is dispensed into between two and ten multi-well plates. Each “Min” flask is pooled into one “Max” flask that is examined for uniform concentration before the pooled cell culture medium is dispensed into multi-well plates. The number of plates dispensed into is typically equal to the number of “Min” flask that are pooled together. This approach is further illustrated in FIG. 43B. As shown, four “Min” flasks 4302 are pooled into one “Max” flask 4304. A sample of cells is withdrawn to determine the cell concentration. The 40 mL of cell culture medium in “Max” flask 4304 are then evenly dispensed into four multi-well plates 4302, resulting in each plate containing 10 mL of cell culture medium.

In the third case, once cell line is dispense into more than ten multi-well plates. For example, 20 “Min” flasks can be dispensed into two “Max” flasks. Five mL of cell culture medium is dispensed from each “Min” flask into each of the “Max” flasks. Using this technique, the two “Max” flasks contain the same concentrations of cells. Thus, only one of the “Max” flasks needs to be examined for uniform concentration. The contents of the two “Max” flasks are then dispensed evenly into 20 multi-well plates. To further illustrate this method, FIG. 43C shows 5 mL from each “Min” flask 4300 being dispensed into each “Max” flask 4304. Following this procedure for 20 “Min” flasks 4300, two “Max” flasks 4304 are filled to their maximum capacities of 100 mL each. The 100 mL of cell culture medium from “Max” flasks 4304 is then evenly distributed into 20 multi-well plates 4302 so that each plate contains 10 mL of cell culture medium.

Cell lines are pooled into “Max” flasks to obtain one cell concentration value that includes only one error. If values for the cell concentrations of each “Min” flask were recorded, then each value would have an associated error and the error would compound. However, by pooling the cell cultures into one “Max” flask one uncompounded error is obtained.

Alternatively, the cells from the “Min” flask could be pooled into one large flask, with a maximum holding capacity of, e.g., 5 L, and tested to ensure the cells are of uniform concentration. This would eliminate the need for distributing the cells into multiple “Max” flasks prior to dispensing the cells into multi-well plates. However, the reasons that this approach is generally not employed relate to the difficulties of keeping such a large flask sterile. The flask would either need to be disposed of between cell lines or need to be washed. This would require a system for removing the flasks or an additional washing station.

In certain embodiments, as discussed briefly above, the transfer of the cell culture or other fluid from a flask to a container such as a multi-well plate (or the single-well plate of FIG. 50A), may be done directly from the flask to the container, without the use of an intermediate container. Other systems utilize a secondary container, which requires either manual replacement of the containers, or the cleaning of reusable containers, which leads to the risk of contamination.

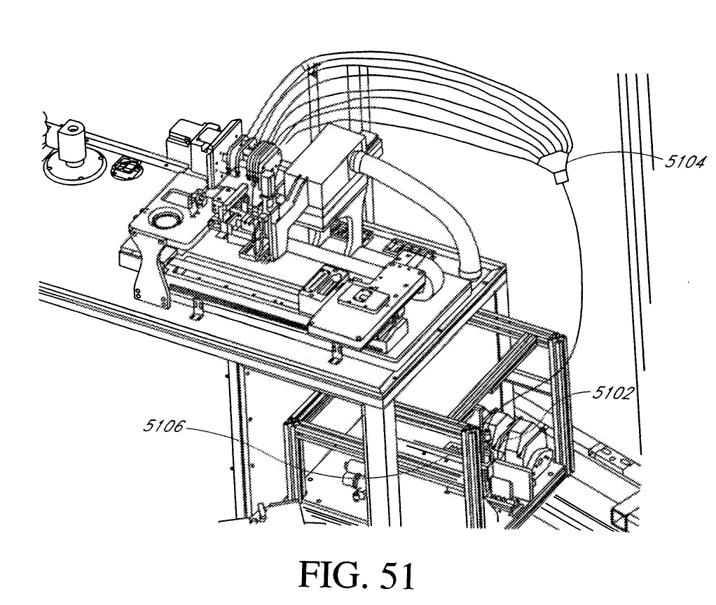

FIG. 51 illustrates such an embodiment, in which fluids are pulled from a flask (i.e., such as the pooling flasks discussed with respect to FIG. 43) via a single tip 5102, and into a manifold 5104, which in the illustrated embodiment is an eight-way manifold. The splitting of the output from the flask into multiple outputs, as shown, increases the rate at which fluid can be dispensed into multi-well plates. In other embodiments, more or less outputs may be used. The tips and tubings of the described dispensing apparatus may be rinsed with multiple cleaning reagents before and after the dispensing of fluid from the flask. This cleaning may be done at a cleaning station 5106.

Accordingly, the invention also provides a dispensing method that includes pooling separate first cell culture media from m source cell culture containers in n destination containers to produce pooled cell culture media in which m is an integer greater than one, and n is an integer greater than zero and less than m. This method also includes transferring selected volumes of the pooled cell culture media from the daughter cell culture containers into selected wells of p multi-well containers in which p is an integer greater than zero to thereby dispense cell culture medium aliquots having substantially uniform cell concentrations.

In one embodiment, scheduler software groups functions into discrete requests, based on the various tasks discussed above. In a particular embodiment, these requests may comprise, for example, passaging, expansion, pooling, plating, profiling, and harvesting. For example, a passaging request instructs the system to take one flask and split off the flask into a separate flask once the flask reaches a particular cell density. An empty flask is filled with the correct amount of cell media and the cells are split from the old flask to the new flask to achieve the desired density. This process occurs continuously as needed to keep the cell lines refreshed with new nutrients. An expansion request takes a flask from passaging and splits it into the required number of flasks. A pooling request pools the expanded flasks into the minimum number of flasks, and adjusts the density to the desired plating density. A plating request instructs the system to dispense the cells directly to a flask based upon user-defined criteria (for example, number of cells per well, well volume, or plate format). Alternately, expanded plates may be harvested directly to an external flask, permitting the collection of a large amount of cells from a cell line. These requests may be batched for multiple cell lines to increase the overall efficiency of the system, and ease data tracking.

### V. EXAMPLES

It is understood that this example and embodiments described herein are for illustrative purposes only and are not intended to limit the scope of the claimed invention. It is also understood that various modifications or changes in light the examples and embodiments described herein will be suggested to persons skilled in the art and are to be included within the spirit and purview of this application and scope of the appended claims.

A. Example Systems

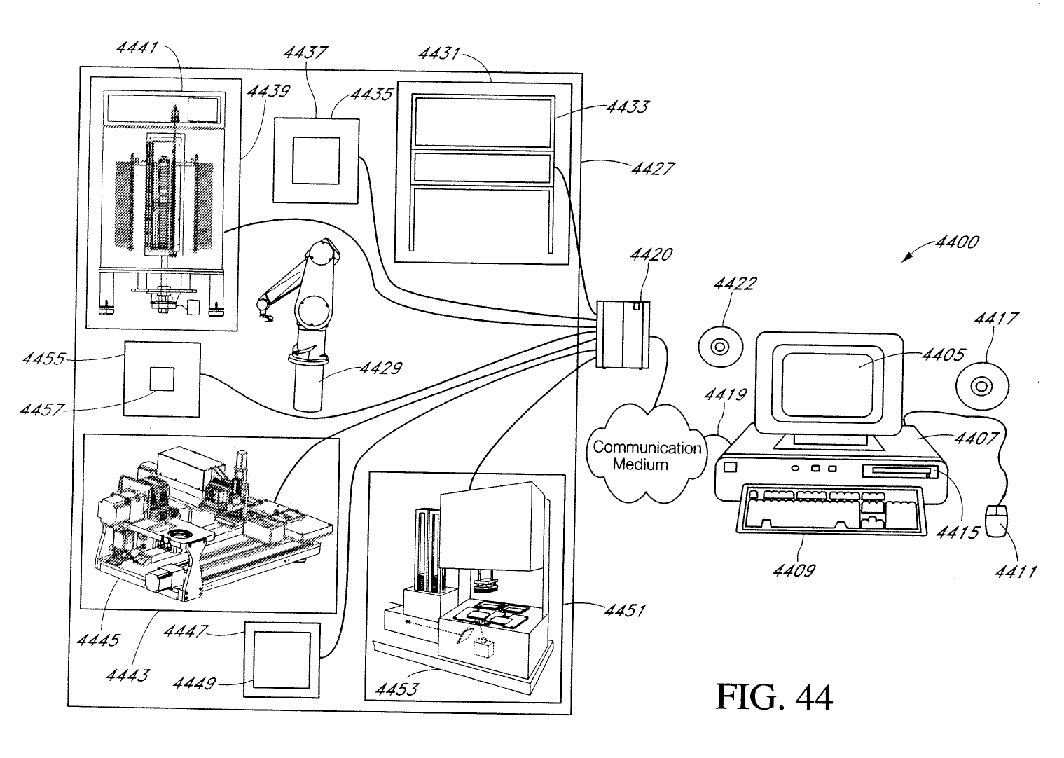

FIG. 44 is a schematic showing an exemplary compound profiling system including an information appliance in which various aspects of the present invention may be embodied. As will be understood by practitioners in the art from the teachings provided herein, the invention is optionally implemented in hardware and software. In some embodiments, different aspects of the invention are implemented in either client-side logic or server-side logic. As will also be understood in the art, the invention or components thereof may be embodied in a media program component (e.g., a fixed media component) containing logic instructions and/or data that, when loaded into an appropriately configured computing device, cause that apparatus or system to perform according to the invention. As will additionally be understood in the art, a fixed media containing logic instructions may be delivered to a viewer on a fixed media for physically loading into a viewer's computer or a fixed media containing logic instructions may reside on a remote server that a viewer accesses through a communication medium in order to download a program component.

FIG. 44 shows information appliance or digital device 4400 that may be understood as a logical apparatus (e.g., a computer, etc.) that can read instructions from media 4417 and/or network port 4419, which can optionally be connected to server 4420 having fixed media 4422. Information appliance 4400 can thereafter use those instructions to direct server or client logic, as understood in the art, to embody aspects of the invention. One type of logical apparatus that may embody the invention is a computer system as illustrated in 4400, containing CPU 4407, optional input devices 4409 and 4411, disk drives 4415 and optional monitor 4405. Fixed media 4417, or fixed media 4422 over port 4419, may be used to program such a system and may represent a disk-type optical or magnetic media, magnetic tape, solid state dynamic or static memory, or the like. In specific embodiments, the aspects of the invention may be embodied in whole or in part as software recorded on this fixed media. Communication port 4419 may also be used to initially receive instructions that are used to program such a system and may represent any type of communication connection. Optionally, aspects of the invention are embodied in whole or in part within the circuitry of an application specific integrated circuit (ACIS) or a programmable logic device (PLD). In such a case, aspects of the invention may be embodied in a computer understandable descriptor language, which may be used to create an ASIC, or PLD.

FIG. 44 also includes work perimeter 4427, which includes robotic gripping device 4429, cell passaging station location 4431 (including cell passaging station 4433), cell counting station location 4435 (including cell counting device 4437), incubation station location 4439 (including incubation device 4441), cell culture plating station location 4443 (including dispensing device 4445), test compound or reagent storage station location 4447 (including test compound or reagent storage device 4449), assaying component station location 4451 (including assaying component 4453), and concentration station location 4455 (including concentration station 4457). It will be appreciated that although only a single work perimeter is depicted in FIG. 44, the system components are optionally distributed in more than one work perimeter that each include a robotic gripping device. It will also be appreciated that other components can also be included, such as fermentors, centrifuges, etc. These system components are typically operably connected to information appliance 4400 directly or via server 4420. During operation, fluid removal station 2524 typically removes fluids from selected wells of multi-well containers positioned and retained on a positioning device of fluid removal station 2524, e.g., as part of a process to clean the containers, and robotic gripping component 2529 moves the containers between the components of multi-well container processing system 2527.

The compound profiling system depicted in FIG. 44 provides for automated storage and retrieval of cell cultures. In particular, this system is optionally configured to store, e.g., hundreds or thousands of cell culture bottles or flasks in a randomly accessible carousel that is enclosed in a temperature, humidity, and CO₂ controlled environment within incubation device 4441. In one embodiment, for example, a system of the invention is configured to store up to 1458 flasks and plates and 108 compound plates. Sterile conditions are also maintained. Robotic gripping device 4429 is designed to retrieve and place the culture bottles at the various workstations shown. After processing, the robotic gripping device 4429 typically places the culture bottles back in, e.g., incubation device 4441. Bar codes on the bottles and a reader on incubation device 4441 maintain the bottle location database, which is typically included as part of information appliance 4400.

Automated cell passaging station 4433 accepts culture bottles from either robotic gripping device 4429 or a human. The bottle is set on a tilting/agitating table or cell culture dissociator of cell passaging station 4433. The bottle is tilted so that all liquid can be aspirated from the bottom corner. A stainless steel cannula is inserted to the bottom of the bottle. For adherent cell lines, all the media is typically aspirated to waste. A wash buffer is then dispensed. The bottle is agitated such that the wash buffer covers all cells. The buffer is aspirated to waste. A trypsin solution (or comparable liquid) is then added to release the cells. The culture bottle is agitated and placed in the temperature controlled incubation device 4441 for up to 30 minutes. Intermittent agitation is optional. Trituration of the sample to achieve a single cell suspension is optional. All but about 1/10th of the original solution is aspirated to waste. Fresh media is then added to the culture bottle to bring the volume back up to the specified growth volume. When an aliquot is scheduled to be frozen, a small sample is extracted to a small, bar coded vessel and moved to an online freezer (not shown). The bar code is read and a database is updated with what is stored in the freezer. Additional options available in the process include, e.g., automated concentration of the cells to remove trypsin reagent, resuspension of cells, and/or transfer to a fresh bottle, if necessary. For non-adherent cells, the washing and trypsinizing steps can be omitted. In the automated system, all cell cultures within the system are automatically passaged per a pre-defined schedule, and subject to monitoring of cell health and density status.

Upon operator command, some number of cell lines can be scheduled for expansion for use in a profiling assay. In certain embodiments, the volume of cells is grown up to one half to one liter. During the scheduled passaging step, the cells that would otherwise be discharged to waste are saved and concentrated in automated concentration station 4457. Here the cells are mildly centrifuged, the trypsin is aspirated off to waste, and fresh media is triturated on the pellet to re-suspend. This re-suspension is then pumped into the final volume of media desired. The cells are counted before this final dispense using cell counting device 4437. The growth parameters are automatically set from this initial count. The liter bottle is mildly agitated before being stored in another automated incubator (not shown) for approximately 1.5 weeks.

When the half-liter expanded culture is ready, it is moved to automated concentration station 4457. The bottle is set into a pre-balanced centrifuge. Here, the media is aspirated to waste, and a wash buffer is then dispensed. The bottle is agitated such that the wash buffer covers all cells. The buffer is aspirated to waste. A trypsin solution (or comparable liquid) is then added to release the cells. The culture bottle is agitated and placed in temperature controlled incubation device 4441 for up to 30 minutes. Intermittent agitation is optional. Trituration of the sample to achieve a single cell suspension is optional. After a few minutes the bottle is mildly centrifuged and the trypsin is drawn off. New media is dispensed and triturated until a single cell suspension is achieved. For suspension cells, the trypsinization steps can be omitted. Cells are subject to a monitor for single cell suspension, which once achieved, are subject to counting using counting device 4437. The correct dilutions are made to achieve desired cell density. Then, the cell culture is pumped off into dispensing device 4445 and plated into 384 or 1536 assay plates. These plates are then lidded and stored in an incubator.

Assays are typically performed using assaying component 4453. In certain embodiments, instead of hundreds of thousands of compounds run against one assay, hundreds of compounds are typically run against 30 or 40 assays. The assay plates have already been plated with cells before they are placed on the assaying component 4453, as described above. The system includes a large amount of bulk dispensing capability to handle the large number of reagents needed to run a diverse collection of assays. In addition, multiple plate readers are typically used to handle diverse readouts. These readers are optionally included as a part of assaying component 4453 or are included at other stations in the system. Compound carousels are generally not needed since the compound input can be done with one or more static hotels (e.g., 1, 2, 3, 4, 5, or more static hotels) in some embodiments. Compound carousels are typically not needed because a set of compounds will only be on the system for a relatively short period of time. In other systems, the compounds can sit in these systems for six months or more. Further, without incubation devices, the compound plates typically undergo water retention into the DMSO solvent of the compounds and the compounds degrade when stored above 4° C. for long periods of time. In the compound profiling systems of the invention, the compound plates usually stay in the system for a maximum time of about two weeks and are generally returned to a controlled environment off the system when the screens are complete. In addition, the system typically runs compound dilution series in each test.

The integrated control system manages all of the processes. Cell counting is performed periodically to adaptively adjust cell growth parameters. Operator and scientific input is generally minimal. Output data is processed and integrated with the data pipeline of the system. As additional options, the filtering and storage of conditioned (used) media is provided for in certain embodiments.

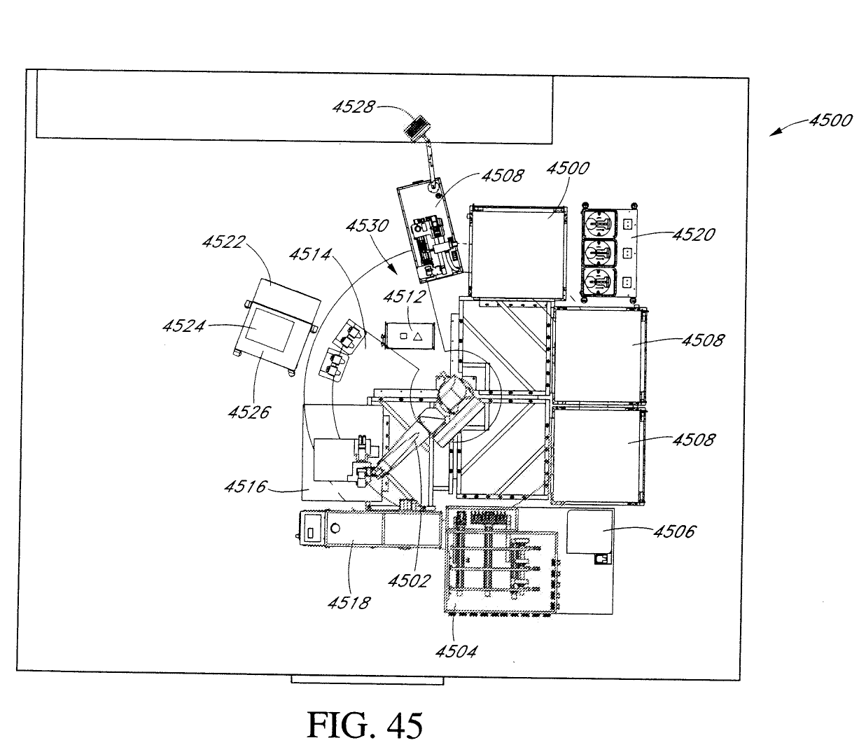

FIG. 45 schematically shows another representative compound profiling system from a top view according to one embodiment of the invention. As shown, system 4500 includes robotic gripping device 4502 disposed within a work perimeter. The work perimeter includes cell passaging station 4504, cell counter 4506, incubation devices 4508, dispensing device 4510, cell J box 4512, static hotel compound library station 4514, detection component 4516, pin tool station 4518, and pin tool pumps 4520. Detection component 4516 typically includes a microscope and multi-well plate readers (e.g., a an ACQUEST™ workstation (Molecular Devices Corp., Sunnyvale, Calif., USA)). As additionally shown, system 4500 further includes electrical enclosure 4522, transformer 4524, and controllers 4526. System 4500 also includes computer 4528. One point of access to the work perimeter of robotic gripping device 4502 is provided by work cell entry 4530.

B. Dispense Head Coils

As described above, the dispense heads of the dispensing devices of the invention include coiled conduit structures in certain embodiments. To adequately compensate for heat loss, the conduit included in a coiled structure must generally be of a minimum length depending on, e.g., the outer diameter (OD) and the wall thickness of the conduit. This example illustrates the calculations that were performed to arrive at the coiled structure length of about 167 mm for a conduit referred to herein. The calculations are as follows:

| Flow | | | |
|---|---|---|---|
| Volumetric flowrateMass FlowrateLine velocityReynolds number | 0.0000010.00099720.9745803660.95789791 | m\hat{ }3/sec kg/sec m/sec dimensionless | ${R}_{e} = \frac{ρVD}{μ}$ |
| Energy Balance | | | |
| Tempereture changeSpecific heatHeat transferred | 2910.0289188 | deg K Kj/kg K KJ/sec | $Q = \dot{m} {c}_{p} Δt$ |
| Transfer temp diffs | | | |
| Initial temp differenceFinal temp differenceLog mean temp diff | 33413.74268723 | Flow vs medium Flow vs medium | $Δt = \frac{{Δt}_{1} - {Δt}_{2}}{ln ( \frac{{Δt}_{1}}{{Δt}_{2}} )}$ |
| Conductive resistance | | | |
| Wall thicknessWell conductivityConductive Resistance | 0.000396875162.48047E−05 | | ${R}_{cond} = \frac{{Wall}_{thick}}{K}$ |
| Tube side heat transfer coefficient | | | |
| Nusselth insideInside resistance | 4.12125.3280840.000470516 | Coulson 425 | $Nu = \frac{hd}{K} = 4.1$ |
| Shell side heat transfer coefficient | | | |
| PrandtlAgitationConstantPrandtl exponentAgitation exponentNusseltNusselt dvh shell sideShell side resistance | 1.1026984130.7684898520.870.330.620.7634263530.05089.4676890190.105622396 | Coulson 497 | $\frac{{h}_{o} {d}_{v}}{k} {( \frac{{μ}_{s}}{μ} )}^{0.14} = 0.87 {( \frac{{C}_{p} μ}{k} )}^{1 / 3} {( \frac{{L}^{2} N ρ}{μ} )}^{0.62}$ |
| Fouling Factors | | | |
| Shell side resistance | 0 | | |
| tube side resistance | 0 | | |
| Heat Exchange Design | | | |
| 1/U = sum of thermal resistancesU = overall heat transfer coefficientA = Heat transfer areaLength of tube | 0.1061177169.4234971680.0002233040.166466197 | | $Q = UA Δt$ |

C. Baculovirus/Insect Cell Automation System

### 1. System Components

A baculovirus/insect cell culture system can include the following components integrated into a fully automated robotic instrument capable of 24/7 operation:

### 1—Staubli RX130L Robot

1—Single cell substructure, mounts and system spine
1—Commercial Systems “Director” scheduling software
1—System controls center
3—Incubators with 486 position flask/plate carousels and incubation/refrigeration
2—Static flask/plate holding “hotels”

### 1—Wave Bioreactor 200

1—Cell counter real time feedback loop for Wave Bioreactor

### 1—BD FACSArray

1—Customized Centrifuge for flasks/plates
1—Customized Tecan Freedom EVO station

### 1—Cell Culture Dissociator (TC Dispenser)

### Consumable Components:

n—Baculovirus/insect cell culture flasks
n—24 well plates
n—96 well plates
n—Reagents, media, cells
2. System Layout

### 3. Automation Process Flow

The process flow is written below in the style of one plate processed at one time for the sake of clarity. However, parallel processing is typically implemented throughout the process.

### A. Set Up

User inputs:
1. Five empty 96 well plates/week for cell count and viral titration assay into static hotel.
2. 288 empty cell culture flasks (“Flasks”) into Incubators/week.
3. Four 24 well cell culture plates into Incubator/week.
4. Sufficient media for Wave Bioreactor.
5. DNAs and GeneJuice onto Tecan in two 96 well plates, for 96 conditions/week.
6. Media in trough on Tecan, 70 ml.
7. Labeled antibody and buffer in one tube each onto Tecan.

Verify:
8. Wave Bioreactor has adequate cells (2×10⁶ cells/ml) and media.
9. Cell Culture Station has sufficient media, cleaning fluids and empty waste.

### B. Week 1: Transfection and First Viral Production

1. Robot transports 96 well plate from Static Hotel to Cell Culture Station.
2. Cell Culture Station dispenses 200 μl Cell Stock into one well of a 96 well plate.
3. Robot transports 96 well plate to FACSArray for accurate count verification and viability determination.
4. Robot transports 96 well plate back to Static Hotel.
5. Adjust Cell Stock concentration in Wave Bioreactor to 2×10⁶ cells/ml with media, if necessary.
6. Robot transports four empty 24 well cell culture plates to Cell Culture Station.
7. Cell Culture Station dispenses 0.5 ml/well of Cell Stock to four 24 well plates.
8. Robot transports 24 well plates to Incubator to adhere for (minimally) 1 hr.
9. Tecan aspirates 25 μl DNA from one well of a 96 well plate and dispenses into corresponding GeneJuice well (25 μl) plate with triturate. Incubate RT 0.5 to 0.75 hr.
10. Robot retrieves four 24 well plates of cells from Incubator and transports to Tecan.
11. Tecan removes media from 24 well plates to waste.
12. Tecan mixes 200 μl media with 50 μl DNA/GeneJuice and adds to cells in 24 well plates. Cells are now termed Transfected.
13. Return 24 well plates of Transfected cells to Incubator and incubate 5 hr.
14. Robot retrieves Transfected cells in 24 well plates from Incubator and transports to Tecan.
15. Tecan removes media to waste.
16. Tecan adds 500 μl fresh media with antibiotics.
17. Robot returns 24 well plates to Incubator (27° C.).
18. Incubate 5 days.
   C. WEEK 2: First round of Viral Amplification
1. Robot transports 24 well plates containing Transfected cells from Incubator to Tecan.
2. Robot transports empty Flask to Cell Culture Station.
3. Cell Culture Station dispenses 30 ml of 2×10⁶ Cell Stock/ml into Flask.
4. Flask of Cell Stock transported to Tecan.
5. Tecan aspirates all supernatant, approximately 0.5 ml, from one well of 24 well plate.
6. Entire supernatant is used to inoculate Flask of Cell Stock (30 ml).
7. The Flask is now termed Infected.
8. Return Infected Flask to Incubator and incubate 5 days.
9. Repeat with 95 other 24 well plate supernatants and Flasks.
10. Robot transports empty 24 well plates to Incubator for operator removal.

### D. WEEK 3: Second Round of Amplification and Archiving

1. Robot transfers two empty Flasks to Cell Culture Station.
2. Cell Culture Station dispenses 30 ml of 2×10⁶ Cell Stock/ml into one Flask.
3. Robot transports infected Flask from Incubator to Centrifuge.
4. Centrifuge spins Flask at 100 g for 10 min.
5. Robot transports ‘spun’ Flask from Centrifuge to Cell Culture Station.
6. Cell Culture Station transfers 1 ml supernatant from ‘spun’ Flask to a Flask containing fresh Cell Stock for infection.
7. Remaining supernatant (˜29 ml) transferred to empty Flask, termed Supernatant Flask.
8. Old Flask transported to waste chute for disposal.
9. Robot transports freshly Infected Flask to Incubator, incubate 5 days.
10. Robot moves Supernatant Flask to Archive Incubator for later titer determination and archiving.
11. Repeat with 95 other Infected Flasks.

The process is repeated up to a total of three amplification cycles.

### E. Determination of Supernatant Viral Titer

1. Supernatant Flasks are transported from Archive Incubator to the Tecan.
2. Robot transports 96 well plate from Static Hotel to Tecan.
3. Robot transports 96 well plate from Static Hotel to Cell Culture Station.
4. Tecan transfers sample of supernatant (200 μl) from Supernatant Flask to empty 96 well plate, termed the Viral Plate.
5. Supernatant Flask returned to Incubator.
6. After x number of supernatants have been transferred, the following assay steps will be performed:
   a. Cell Culture Station dispenses 200 μl/well of counted and adjusted Cell Stock into a 96 well plate, termed the Cell Plate.
   b. Robot transports Cell Plate to Centrifuge.
   c. Centrifuge spins Cell Plate at 1000 g for 10 min.
   d. Robot transports Cell Plate to Tecan which aspirates supernatant to waste.
   e. Tecan transfers viral supernatant (200 μl) from the Viral Plate to the Cell Plate.
   f. Incubate for 1 hr at room temperature.
   g. Tecan adds tagged antibody (100 μl) to cells.
   h. Incubate one hour room temperature.
   i. Robot transports plate to Centrifuge.
   j. Spin plate 1000 g for 10 min.
   k. Robot transports plate to Tecan which aspirates supernatant to waste.
   l. Tecan adds 200 μl buffer.
   m. Robot transports plate to FACSArray.
   n. FACSArray samples and measures each well.
   o. A viral titer is assigned to each archived Supernatant Flask.
   p. Robot moves two 96 well plates to Static Hotel for operator removal.
   q. Operator removes used 96 well plate plates from system.

### F. Output

1. Supernatant Flasks may be removed from the system at any time to another archive.
2. Supernatant Flasks may be removed from the system at any time to Piccolo.
3. Supernatant Flasks may be re-arrayed into deep well 96 well plates for archiving or compatibility with Piccolo.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes.

What is claimed is:

1. An automated cell culture and passaging system, comprising:
an incubation device adapted to facilitate growth of cells in cell culture containers; and
an assaying component configured to perform an assay on cells from said cell cultures, wherein the incubation device is adapted to permit the cells from the cell culture to be directly or indirectly delivered to the assay device without the need for human intervention.

2. The system of claim 1, wherein the assaying component comprises:
a test reagent source region structured to support at least one test reagent source container;
an assaying region structured to support at least one cell sample container; and,
a material transfer device that is configured to transfer at least one test reagent from the test reagent source container to the cell sample container when the test reagent source container is supported in the test reagent source region and the cell sample container is supported in the assaying region.

3. The system of claim 2, additionally comprising a controller, which controller comprises a logic device;

4. The system of claim 3, wherein the controller is operably connected to the material transfer device, and wherein the logic device comprises logic instructions that direct movement of the material transfer device between the test reagent source region and the assaying region.

5. The system of claim 3, wherein either or both of the cell sample container and the test reagent source container are multi-well containers.

6. The system of claim 3, wherein the test reagents comprise one or more reagents selected from the group consisting of compounds, proteins, nucleic acids, virus particles, and bacteriophage.

7. The system of claim 6, wherein the test reagents comprise nucleic acids selected from the group consisting of siRNA molecules, antisense RNA molecules, cDNAs, and vectors.

8. The system of claim 6, wherein the test reagents comprise proteins selected from the group consisting of enzymes, antibodies, and regulatory proteins.

9. The system of claim 6, wherein the test reagents comprise virus particles selected from the group consisting of baculovirus, retrovirus, lentivirus, and adenovirus.

10. The system of claim 3, further comprising at least one detector configured to detect one or more detectable signals produced in the cell sample container.

11. The system of claim 3, wherein the material transfer device comprises a non-pressure-based material transfer probe.

12. The system of claim 11, wherein the non-pressure-based material transfer probe comprises a pin tool.

13. The system of claim 12, wherein the material transfer device comprises at least one chassis and the pin tool comprises a support structure having at least one attachment feature that removably attaches to the chassis.

14. The system of claim 13, wherein the logic device comprises logic instructions that directs the material transfer device to attach and/or detach the pin tool to or from the chassis.

15. The system of claim 13, wherein the pin tool comprises a pin tool head having a rotational adjustment feature such that the pin tool head is capable of rotating relative to the support structure along one or more axes.

16. The system of claim 3, wherein the test reagent source region and/or the assaying region comprises a container positioning device, which container positioning device comprises at least one container station that is structured to position at least one container relative to the material transfer device.

17. The system of claim 16, wherein the container station is structured to position at least one multi-well container that comprises 6, 12, 24, 48, 96, 192, 384, 768, 1536, 3456, 9600, or more wells.

18. The system of claim 16, wherein the container station is structured to rotate relative to the material transfer device.

19. The system of claim 3, further comprising at least one material transfer probe washing station that comprises at least one wash reservoir structured to wash the non-pressure-based material transfer probe.

20. The system of claim 19, wherein the wash reservoir comprises at least one mount to position the non-pressure-based material transfer probe relative to the wash reservoir when the non-pressure-based material transfer probe is washed and/or when the non-pressure-based material transfer probe is separated from a chassis of the material transfer device.

21. The system of claim 1, further comprising a decontamination device that comprises:
a first chamber that comprises a system component disposed therein;
a second chamber that communicates with the first chamber such that one or more containers are capable of being translocated between the first and second chambers; and,
a decontamination component that communicates at least with the second chamber, which decontamination component is configured to substantially decontaminate one or more surfaces of the containers when the containers are disposed in the second chamber.

22. The system of claim 21, wherein the system component comprises a cell culture dissociator, a material handling component, and/or a container positioning device.

23. The system of claim 21, further comprising a translocation mechanism that is structured to translocate at least one container at least between the first and second chambers.

24. The system of claim 21, wherein the first chamber comprises a substantially sterile environment.

25. The system of claim 21, wherein the second chamber comprises an ante-chamber.

26. The system of claim 21, wherein the decontamination component comprises at least one radiation source that irradiates the surfaces of the containers to substantially decontaminate the surfaces when the containers are disposed in the second chamber.

27. The system of claim 21, wherein the decontamination component comprises at least one temperature modulator that modulates temperatures in the second chamber to substantially decontaminate the surfaces when the containers are disposed in the second chamber.

28. The system of claim 21, wherein the decontamination component comprises at least one decontamination fluid mister that sprays a mist of a decontamination fluid onto the surfaces of the containers to substantially decontaminate the surfaces when the containers are disposed in the second chamber.

29. The system of claim 21, wherein the decontamination component comprises at least one gas source that flows gas into the second chamber at velocities that are sufficient to substantially remove at least one contaminant from one or more surfaces of the containers when the containers are disposed in the second chamber.

30. The system of claim 29, wherein the gas comprises air.

31. The system of claim 1, further comprising a controller and one or more additional system components operably connected to the controller, which additional system components are selected from the group consisting of: a robotic gripping device, a material handling component, a cell counting device, a centrifuge, a detector, a freezer, a fermentor, a waste container, a filtration device, a lid processing device, a transfer station, an incubation device, a colony picking device, a high content imaging device, a pin tool drying or blotting station, a cell dissociator, and a container storage device.

32. The system of claim 31, further comprising at least one container location database operably connected to the controller, which container location database comprises entries that correspond to locations of containers in the system.

33. The system of claim 1, further comprising a material handling component, wherein the material handling component comprises at least one fluidic material transfer component that is configured to transfer fluidic materials to and/or from containers positioned in one or more components of the system.

34. The system of claim 33, wherein the fluidic material transfer component is configured to transfer cell culture media among cell culture sample vessels, cell culture flasks, and/or multi-well containers.

35. The system of claim 34, further comprising a controller, which controller comprises a logic device, wherein the logic device comprises at least one logic instruction for:
pooling separate first cell culture media from m first cell culture containers in n second containers to produce pooled cell culture media using the fluidic material transfer component, wherein m is an integer greater than one, and wherein n is an integer greater than zero and less than m; and,
transferring selected volumes of the pooled cell culture media from the n second containers into selected wells of p multi-well containers using the fluidic material transfer component, wherein p is an integer greater than one.

36. The system of claim 35, further comprising at least one detection component operably connected to the controller, which detection component is configured to detect a concentration of cells in or from the pooled cell culture media.

37. The system of claim 33, wherein the fluidic material transfer component comprises a dispensing device that comprises:
a conduit that comprises an inlet and an outlet that fluidly communicate with one another;
a fluid source that fluidly communicates with the inlet of the conduit;
a fluid conveyance device operably connected to the conduit and/or to the fluid source, which fluid conveyance device is configured to convey at least one fluidic reagent through the conduit from the fluid source; and,
a thermal regulation component that thermally communicates with at least a portion of the conduit, which thermal regulation component is configured to selectively regulate a temperature of the fluidic reagent when the fluidic reagent is conveyed through the conduit from the fluid source.

38. The system of claim 37, further comprising a fluid source storage device that stores the fluid source at a selected temperature.

39. The system of claim 38, wherein the selected temperature is about 4° C.

40. The system of claim 37, further comprising at least one dispense head that comprises at least a segment of the conduit.

41. The system of claim 40, wherein the segment of the conduit comprises a coiled structure.

42. The system of claim 40, further comprising a plurality of conduits, wherein the dispense head comprises one or more segments of each of the conduits.

43. The system of claim 42, further comprising a plurality of fluid sources, wherein each of the conduits fluidly communicates with a different fluid source.

44. The system of claim 40, wherein the dispense head comprises at least one chamber that comprises the segment of the conduit, which chamber comprises at least one opening that fluidly communicates with the thermal regulation component, which thermal regulation component is configured to flow at least one fluidic material having a selected temperature into the chamber such that when the fluidic reagent is flowed through the segment of the conduit, the fluidic reagent substantially attains the selected temperature.

45. The system of claim 44, wherein the fluidic material comprises an antifreeze solution.

46. The system of claim 44, wherein the selected temperature is about 37° C.

47. The system of claim 44, wherein the thermal regulation component comprises at least one fluidic material recirculation bath that substantially maintains the fluidic material at the selected temperature.

48. The system of claim 1, further comprising at least one high throughput processing station that comprises at least one rotational robot that comprises a reach that defines a work perimeter associated with the rotational robot, wherein at least the cell culture device is within the reach of the rotational robot.

49. The system of claim 1, further comprising a robotic arm that can transfer cell culture containers between the cell culture device and the assay device.

50. The system of claim 49, further comprising at least a second robotic arm.

51. The system of claim 1, wherein the automated cell culture passaging system can split or subculture two or more cell lines without human intervention.

52. The system of claim 51, wherein the automated cell culture passaging system can split or subculture 25 or more cell lines without human intervention

53. The system of claim 51, wherein the system further comprises a cell dissociator comprising:
a container holder comprising a container receiving area that is structured to receive at least one cell culture container;
a moving mechanism operably connected to the container holder, which mechanism is configured to move the container holder between a first position and a second position; and
a stop that limits movement of the container holder by the moving mechanism;
a material handling component; and
a controller operably connected to the cell culture dissociator and to the material handling component, which controller comprises a logic device that comprises logic instructions that direct the moving mechanism to move the container holder at a selected rate, and the material handling component to dispense material into, and/or to remove material from, the cell culture container when the cell culture container is disposed in the container receiving area.

54. The system of claim 51, wherein:
the moving mechanism comprises a rotational mechanism, which rotational mechanism is configured to rotate the container holder about an axis;
the stop limits angular displacement of the container holder by the rotational mechanism; and
the logic instructions direct the rotational mechanism to rotate the container holder at a selected rate.

55. The system of claim 54, wherein the rotational mechanism comprises a counterweight that counters a weight of the container holder when the rotational mechanism rotates the container holder.

56. The system of claim 54, wherein the cell culture dissociator comprises multiple container holders, which container holders are symmetrically positioned relative to a rotatational axis such that the container holders counterbalance one another.

57. The system of claim 54, wherein the rotational mechanism comprises a first stop that limits the angular displacement of the container holder in a first direction, and a second stop that limits the angular displacement of the container holder in a second direction that is opposite to the first direction.

58. The system of claim 54, wherein the selected rate is an angular velocity of at least 0.25 rev/s when the stop is contacted.

59. The system of claim 54, wherein the container holder decelerates at a rate of at least 1.0 rev/s² when the stop is contacted.

60. The system of claim 54, wherein the container holder is structured to receive a cell culture container that comprises a top wall, which top wall comprises a major axis and a minor axis, and the rotational mechanism rotates the container holder in a first direction and an opposite second direction that are parallel to a minor axis of the top wall of the cell culture container.

61. The system of claim 54, wherein the container holder is structured to receive cell culture container that comprises a top wall, which top wall comprises a major axis and a minor axis, and the rotational mechanism rotates the container holder in a first direction and an opposite second direction that are parallel to a major axis of the top wall of the cell culture container.

62. The system of claim 54, further comprising at least one container retention component that is movable relative to the container holder, which container retention component is structured to retain the cell culture container in a substantially fixed position relative to the container retention component when the cell culture container is disposed in the container receiving area and the container holder is in a closed position.

63. The system of claim 62, wherein the container holder and the container retention component are coupled to one another via at least one slidable coupling.

64. The system of claim 62, wherein the logic device comprises at least one logic instruction that directs the container holder to close or open.

65. The system of claim 62, wherein the container retention component comprises a container retention plate.

66. The system of claim 62, wherein the container retention component is structured to permit access to the cell culture container when the cell culture container is disposed in the container receiving area and the container holder is in the closed position.

67. The system of claim 54, further comprising a multicontainer holder that comprises a plurality of container holders, which multicontainer holder is not operably connected to the moving mechanism.

68. The system of claim 67, wherein the logic device comprises at least one logic instruction that directs the container holders to close or open.

69. The system of claim 67, further comprising at least one translational mechanism operably connected to the multicontainer holder, which translational mechanism is configured to move the multicontainer holder along at least one translational axis.

70. The system of claim 69, wherein the controller is operably connected to the translational mechanism and comprises at least one logic instruction that directs the translational mechanism to translate the multicontainer holder to one or more selected positions along the translational axis.

71. An automated method of passaging a cell culture and performing an assay, the method comprising:
transferring a portion of a cell culture media located within a source container to a daughter flask;
dispensing at least a portion of the cell culture media located within the daughter container to an assay container; and
performing an assay on the portion of the cell culture media located within the assay container, wherein the steps of transferring a portion of a cell culture media located within the source container to the daughter container, transferring a portion of a cell culture media located within the daughter container to an assay container, and performing the assay are done without human intervention.
